SEQUENCE LISTING (27 sequences)

SEQ ID NO: 1 - protein, 161 aa, Vigna radiata
MTTTLERGFTEEQEALVVKSWNVMKKNSGELGLKFFLKIFEIAPSAQKLFSFLRDSTVPLEQNPKLKPHAVSVFVMTCDSAVQLRKAGKVTVRESNLKKLGATHFRTGVANEHFEVTKFALLETIKEAVPEMWSPAMKNAWGEAYDQLVDAIKYEMKPPSS

SEQ ID NO: 2 - protein, 133 aa, Methylacidiphilum infernorum
MIDQKEKELIKESWKRIEPNKNEIGLLFYANLFKEEPTVSVLFQNPISSQSRKLMQVLGILVQGIDNLEGLIPTLQDLGRRHKQYGVVDSHYPLVGDCLLKSIQEYLGQGFTEEAKAAWTKVYGIAAQVMTAE

SEQ ID NO: 3 - protein, 139 aa, Aquifex aeolicus
MLSEETIRVIKSTVPLLKEHGTEITARMYELLFSKYPKTKELFAGASEEQPKKLANAIIAYATYIDRLEELDNAISTIARSHVRRNVKPEHYPLVKECLLQAIEEVLNPGEEVLKAWEEAYDFLAKTLITLEKKLYSQP

SEQ ID NO: 4 - protein, 145 aa, Glycine max
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAF

SEQ ID NO: 5 - protein, 162 aa, Hordeum vulgare
MSAAEGAVVFSEEKEALVLKSWAIMKKDSANLGLRFFLKIFEIAPSARQMFPFLRDSDVPLETNPKLKTHAVSVFVMTCEAAAQLRKAGKITVRETTLKRLGGTHLKYGVADGHFEVTRFALLETIKEALPADMWGPEMRNAWGEAYDQLVAAIKQEMKPAE

SEQ ID NO: 6 - protein, 1153 aa, Magnaporthe oryzae
MDGAVRLDWTGLDLTGHEIHDGVPIASRVQVMVSFPLFKDQHIIMSSKESPSRKSSTIGQSTRNGSCQADTQKGQLPPVGEKPKPVKENPMKKLKEMSQRPLPTQHGDGTYPTEKKLTGIGEDLKHIRGYDVKTLLAMVKSKLKGEKLKDDKTMLMERVMQLVARLPTESKKRAELTDSLINELWESLDHPPLNYLGPEHSYRTPDGSYNHPFNPQLGAAGSRYARSVIPTVTPPGALPDPGLIFDSIMGRTPNSYRKHPNNVSSILWYWATIIIHDIFWTDPRDINTNKSSSYLDLAPLYGNSQEMQDSIRTFKDGRMKPDCYADKRLAGMPPGVSVLLIMFNRFHNHVAENLALINEGGRFNKPSDLLEGEAREAAWKKYDNDLFQVARLVTSGLYINITLVDYVRNIVNLNRVDTTWTLDPRQDAGAHVGTADGAERGTGNAVSAEFNLCYRWHSCISEKDSKFVEAQFQNIFGKPASEVRPDEMWKGFAKMEQNTPADPGQRTFGGFKRGPDGKFDDDDLVRCISEAVEDVAGAFGARNVPQAMKVVETMGIIQGRKWNVAGLNEFRKHFHLKPYSTFEDINSDPGVAEALRRLYDHPDNVELYPGLVAEEDKQPMVPGVGIAPTYTISRVVLSDAVCLVRGDRFYTTDFTPRNLTNWGYKEVDYDLSVNHGCVFYKLFIRAFPNHFKQNSVYAHYPMVVPSENKRILEALGRADLFDFEAPKYIPPRVNITSYGGAEYILETQEKYKVTWHEGLGFLMGEGGLKFMLSGDDPLHAQQRKCMAAQLYKDGWTEAVKAFYAGMMEELLVSKSYFLGNNKHRHVDIIRDVGNMVHVHFASQVFGLPLKTAKNPTGVFTEQEMYGILAAIFTTIFFDLDPSKSFPLRTKTREVCQKLAKLVEANVKLINKIPWSRGMFVGKPAKDEPLSIYGKTMIKGLKAHGLSDYDIAWSHVVPTSGAMVPNQAQVFAQAVDYYLSPAGMHYIPEIHMVALQPSTPETDALLLGYAMEGIRLAGTFGSYREAAVDDVVKEDNGRQVPVKAGDRVFVSFVDAARDPKHFPDPEVVNPRRPAKKYIHYGVGPHACLGRDASQIAITEMFRCLFRRRNVRRVPGPQGELKKVPRPGGFYVYMREDWGGLFPFPVTMRVMWDDE

SEQ ID NO: 7 - protein, 530 aa, Fusarium oxysporum
MKGSATLAFALVQFSAASQLVWPSKWDEVEDLLYMQGGFNKRGFADALRTCEFGSNVPGTQNTAEWLRTAFHDAITHDAKAGTGGLDASIYWESSRPENPGKAFNNTFGFFSGFHNPRATASDLTALGTVLAVGACNGPRIPFRAGRIDAYKAGPAGVPEPSTNLKDTFAAFTKAGFTKEEMTAMVACGHAIGGVHSVDFPEIVGIKADPNNDTNVPFQKDVSSFHNGIVTEYLAGTSKNPLVASKNATFHSDKRIFDNDKATMKKLSTKAGFNSMCADILTRMIDTVPKSVQLTPVLEAYDVRPYITELSLNNKNKIHFTGSVRVRITNNIRDNNDLAINLIYVGRDGKKVTVPTQQVTFQGGTSFGAGEVFANFEFDTTMDAKNGITKFFIQEVKPSTKATVTHDNQKTGGYKVDDTVLYQLQQSCAVLEKLPNAPLVVTAMVRDARAKDALTLRVAHKKPVKGSIVPRFQTAITNFKATGKKSSGYTGFQAKTMFEEQSTYFDIVLGGSPASGVQFLTSQAMPSQCS

SEQ ID NO: 8 - protein, 358 aa, Fusarium graminearum
MASATRQFARAATRATRNGFAIAPRQVIRQQGRRYYSSEPAQKSSSAWIWLTGAAVAGGAGYYFYGNSASSATAKVFNPSKEDYQKVYNEIAARLEEKDDYDDGSYGPVLVRLAWHASGTYDKETGTGGSNGATMRFAPESDHGANAGLAAARDFLQPVKEKFPWITYSDLWILAGVCAIQEMLGPAIPYRPGRSDRDVSGCTPDGRLPDASKRQDHLRGIFGRMGFNDQEIVALSGAHALGRCHTDRSGYSGPWTFSPTVLTNDYFRLLVEEKWQWKKWNGPAQYEDKSTKSLMMLPSDIALIEDKKFKPWVEKYAKDNDAFFKDFSNVVLRLFELGVPFAQGTENQRWTFKPTHQE

SEQ ID NO: 9 - protein, 122 aa, Chlamydomonas eugametos
MSLFAKLGGREAVEAAVDKFYNKIVADPTVSTYFSNTDMKVQRSKQFAFLAYALGGASEWKGKDMRTAHKDLVPHLSDVHFQAVARHLSDTLTELGVPPEDITDAMAVVASTRTEVLNMPQQ

SEQ ID NO: 10 - protein, 121 aa, Tetrahymena pyriformis
MNKPQTIYEKLGGENAMKAAVPLFYKKVLADERVKHFFKNTDMDHQTKQQTDFLTMLLGGPNHYKGKNMTEAHKGMNLQNLHFDAIIENLAATLKELGVTDAVINEAAKVIEHTRKDMLGK

SEQ ID NO: 11 - protein, 117 aa, Paramecium caudatum
MSLFEQLGGQAAVQAVTAQFYANIQADATVATFFNGIDMPNQTNKTAAFLCAALGGPNAWTGRNLKEVHANMGVSNAQFTTVIGHLRSALTGAGVAAALVEQTVAVAETVRGDVVTV

SEQ ID NO: 12 - protein, 147 aa, Aspergillus niger
MPLTPEQIKIIKATVPVLQEYGTKITTAFYMNMSTVHPELNAVFNTANQVKGHQARALAGALFAYASHIDDLGALGPAVELICNKHASLYIQADEYKIVGKYLLEAMKEVLGDACTDDILDAWGAAYWALADIMINREAALYKQSQG

SEQ ID NO: 13 - protein, 165 aa, Zea mays
MALAEADDGAVVFGEEQEALVLKSWAVMKKDAANLGLRFFLKVFEIAPSAEQMFSFLRDSDVPLEKNPKLKTHAMSVFVMTCEAAAQLRKAGKVTVRETTLKRLGATHLRYGVADGHFEVTGFALLETIKEALPADMWSLEMKKAWAEAYSQLVAAIKREMKPDA

SEQ ID NO: 14 - protein, 169 aa, Oryza sativa subsp. japonica
MALVEGNNGVSGGAVSFSEEQEALVLKSWAIMKKDSANIGLRFFLKIFEVAPSASQMFSFLRNSDVPLEKNPKLKTHAMSVFVMTCEAAAQLRKAGKVTVRDTTLKRLGATHFKYGVGDAHFEVTRFALLETIKEAVPVDMWSPAMKSAWSEAYNQLVAAIKQEMKPAE

SEQ ID NO: 15 - protein, 160 aa, Arabidopsis thaliana
MESEGKIVFTEEQEALVVKSWSVMKKNSAELGLKLFIKIFEIAPTTKKMFSFLRDSPIPAEQNPKLKPHAMSVFVMCCESAVQLRKTGKVTVRETTLKRLGASHSKYGVVDEHFEVAKYALLETIKEAVPEMWSPEMKVAWGQAYDHLVAAIKAEMNLSN

SEQ ID NO: 16 - protein, 147 aa, Pisum sativum
MGFTDKQEALVNSSWESFKQNLSGNSILFYTIILEKAPAAKGLFSFLKDTAGVEDSPKLQAHAEQVFGLVRDSAAQLRTKGEVVLGNATLGAIHVQRGVTDPHFVVVKEALLQTIKKASGNNWSEELNTAWEVAYDGLATAIKKAMT

SEQ ID NO: 17 - protein, 145 aa, Vigna unguiculata
MVAFSDKQEALVNGAYEAFKANIPKYSVVFYTTILEKAPAAKNLFSFLANGVDATNPKLTGHAEKLFGLVRDSAAQLRASGGVVADAALGAVHSQKAVNDAQFVVVKEALVKTLKEAVGDKWSDELGTAVELAYDELAAAIKKAY

SEQ ID NO: 18 - protein, 154 aa, Bos taurus
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 19 - protein, 154 aa, Sus scrofa
MGLSDGEWQLVLNVWGKVEADVAGHGQEVLIRLFKGHPETLEKFDKFKHLKSEDEMKASEDLKKHGNTVLTALGGILKKKGHHEAELTPLAQSHATKHKIPVKYLEFISEAIIQVLQSKHPGDFGADAQGAMSKALELFRNDMAAKYKELGFQG

SEQ ID NO: 20 - protein, 154 aa, Equus caballus
MGLSDGEWQQVLNVWGKVEADIAGHGQEVLIRLFTGHPETLEKFDKFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

SEQ ID NO: 21 - protein, 152 aa, Nicotiana benthamiana
MSSFTEEQEALVVKSWDSMKKNAGEWGLKLFLKIFEIAPSAKKLFSFLKDSNVPLEQNAKLKPHSKSVFVMTCEAAVQLRKAGKVVVRDSTLKKLGATHFKYGVADEHFEVTKFALLETIKEAVPEMWSVDMKNAWGEAFDQLVNAIKTEMK

SEQ ID NO: 22 - protein, 132 aa, Bacillus subtilis
MGQSFNAPYEAIGEELLSQLVDTFYERVASHPLLKPIFPSDLTETARKQKQFLTQYLGGPPLYTEEHGHPMLRARHLPFPITNERADAWLSCMKDAMDHVGLEGEIREFLFGRLELTARHMVNQTEAEDRSS

SEQ ID NO: 23 - protein, 131 aa, Corynebacterium glutamicum
MTTSENFYDSVGGEETFSLIVHRFYEQVPNDDILGPMYPPDDFEGAEQRLKMFLSQYWGGPKDYQEQRGHPRLRMRHVNYPIGVTAAERWLQLMSNALDGVDLTAEQREAIWEHMVRAADMLINSNPDPHA

SEQ ID NO: 24 - protein, 124 aa, Synechocystis sp.
MSTLYEKLGGTTAVDLAVDKFYERVLQDDRIKHFFADVDMAKQRAHQKAFLTYAFGGTDKYDGRYMREAHKELVENHGLNGEHFDAVAEDLLATLKEMGVPEDLIAEVAAVAGAPAHKRDVLNQ

SEQ ID NO: 25 - protein, 183 aa, Synechococcus sp.
MDVALLEKSFEQISPRAIEFSASFYQNLFHHHPELKPLFAETSQTIQEKKLIFSLAAIIENLRNPDILQPALKSLGARHAEVGTIKSHYPLVGQALIETFAEYLAADWTEQLATAWVEAYDVIASTMIEGADNPAAYLEPELTFYEWLDLYGEESPKVRNAIATLTHFHYGEDPQDVQRDSRG

SEQ ID NO: 26 - protein, 118 aa, Nostoc commune
MSTLYDNIGGQPAIEQVVDELHKRIATDSLLAPVFAGTDMVKQRNHLVAFLAQIFEGPKQYGGRPMDKTHAGLNLQQPHFDAIAKHLGERMAVRGVSAENTKAALDRVTNMKGAILNK

SEQ ID NO: 27 - protein, 136 aa, Bacillus megaterium
MREKIHSPYELLGGEHTISKLVDAFYTRVGQHPELAPIFPDNLTETARKQKQFLTQYLGGPSLYTEEHGHPMLRARHLPFEITPSRAKAWLTCMHEAMDEINLEGPERDELYHRLILTAQHMINSPEQTDEKGFSH

(19) United States
(12) Patent Application Publication
(10) Pub. No.: US 2023/0210150 A1
(43) Pub. Date: Jul. 6, 2023

(54) MATERIALS AND METHODS FOR PROTEIN PRODUCTION

(71) Applicant: Impossible Foods Inc., Redwood City, CA (US)

(72) Inventors: Xin Li, Fremont, CA (US); Michelle Mai, Daly City, CA (US); Yiming Chen, Fremont, CA (US); Ranjani Varadan, Fremont, CA (US)

(21) Appl. No.: 17/905,082

(22) PCT Filed: Mar. 1, 2021
(86) PCT No.: PCT/US2021/020356

§ 371 Date: Aug. 26, 2022

(51) Int. Cl. A23L 11/65; A23C 11/10; A23J 1/14; A23J 3/16; A23L 13/40; A23L 33/185

(52) U.S. Cl. CPC A23L 11/65; A23C 11/10; A23J 1/14; A23J 3/16; A23L 13/426; A23L 33/185

(57) ABSTRACT

This document relates to materials and methods for the production of protein. For example, proteins having a low flavor or low color profile and food products comprising the same.

### CROSS-REFERENCE TO RELATED APPLICATIONS

This application claims priority to U.S. Provisional Application Serial Nos. 62/983,558, filed on Feb. 28, 2020 and 62/993,675, filed on Mar. 23, 2020, each of which is incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: 38767_0246WO1.txt, date recorded, Mar. 1, 2021, file size ≈ 56 kilobytes.

### TECHNICAL FIELD

This invention relates to methods for purifying protein, and more particularly to methods for purifying protein to help reduce colors, odors, and flavors that are associated with the source of the protein. This invention also relates to food products including purified protein.

### BACKGROUND

The success of food products that mimic animal derived food products (e.g., cheese or meat) is largely dependent on generating functional protein that can be manipulated and has low-flavor so the source of the protein is not readily identifiable by the flavor profile of the food product mimic. It would be useful to have a method of protein purification that is food-safe and results in minimal undesirable colors, odors, and flavors in the purified protein.

### SUMMARY

This document is based, at least in part, on the production of protein compositions using precipitation.

In one aspect, low flavor protein compositions are provided. Such low flavor protein compositions generally include at least 50% by dry weight of a plurality of plant proteins, fungal proteins, algal proteins, bacterial proteins, protozoan proteins, invertebrate proteins, or a combination thereof; wherein the plurality of plant proteins, fungal proteins, algal proteins, bacterial proteins, protozoan proteins, invertebrate proteins, or combination thereof are substantially aggregated, denatured, or both.

In some embodiments, the low flavor protein composition has a luminance of at least 86 on a scale from 0 (black control value) to 100 (white control value). In some embodiments, the low flavor protein composition has a luminance of at least 88 on a scale from 0 (black control value) to 100 (white control value). In some embodiments, the low flavor protein composition has a luminance of at least 90 on a scale from 0 (black control value) to 100 (white control value).

In some embodiments, the low flavor protein composition has a chroma value of less than 14. In some embodiments, the low flavor protein composition has a chroma value of less than 12. In some embodiments, the low flavor protein composition has a chroma value of less than 10. In some embodiments, the low flavor protein composition has a chroma value of less than 8. In some embodiments, the low flavor protein composition has a chroma value of less than 6.

In some embodiments, the low flavor protein composition comprises less than about 1.2% by dry weight lipids (e.g., less than about 1.0% or less than about 0.5% by dry weight lipids).

In some embodiments, the lipids comprise one or more of a fatty acid, a wax, a sterol, a monoglyceride, a diglyceride, a triglyceride, a sphingolipid, or a phospholipid.

In some embodiments, the plurality of plant proteins, fungal proteins, algal proteins, bacterial proteins, protozoan proteins, invertebrate proteins, or a combination thereof is at least 90% by dry weight soy proteins.

In some embodiments, a low flavor protein composition further includes at least one of a preservative, an antioxidant, or a shelf life extender.

In some embodiments, the preservative, antioxidant, or shelf life extender comprises at least one of 4-hexylresorcinol, acetic acid, ascorbic acid, ascorbyl palmitate, ascorbyl stearate, benzoic acid, butylated hydroxyanisole (a mixture of 2-tertiarybutyl-4-hydroxyanisole and 3-tertiarybutyl-4-hydroxyanisole), butylated hydroxytoluene (3,5-ditertiarybutyl-4-hydroxytoluene), calcium ascorbate, calcium propionate, calcium sorbate, Carnobacterium divergens M35, Carnobacterium maltaromaticum cb1, carnosum 4010, citric acid, a citric acid ester of a monoglyceride or diglyceride, dimethyl dicarbonate, erythorbic acid, ethyl lauroyl arginate, gum guaiacum, iso-ascorbic acid, L-cysteine, L-cysteine hydrochloride, lecithin, lecithin citrate, Leuconostoc, methyl paraben, methyl-p-hydroxybenzoate, monoglyceride citrate, monoisopropyl citrate, natamycin, nisin, potassium acetate, potassium benzoate, potassium bisulfite, potassium diacetate, potassium lactate, potassium metabisulfite, potassium nitrate, potassium nitrite, potassium sorbate, propionic acid, propyl gallate, propyl paraben, propyl-p-hydroxy benzoate, sodium acetate, sodium ascorbate, sodium benzoate, sodium bisulfite, sodium diacetate, sodium dithionite, sodium erythorbate, sodium iso-ascorbate, sodium lactate, sodium metabisulfite, sodium nitrate, sodium nitrite, sodium propionate, sodium salt of methyl-p-hydroxy benzoic acid, sodium salt of propyl-p-hydroxy benzoic acid, sodium sorbate, sodium sulfite, sorbic acid, sulfurous acid, tartaric acid, tertiary butyl hydroquinone, or a tocopherol.

In some embodiments, the low flavor protein composition is in the form of a solution, suspension, or emulsion. In some embodiments, the low flavor protein composition is in the form of a solid or a powder.

In some embodiments, the low flavor protein composition has an average particle size of about 5 µm to about 40 µm in the largest dimension. In some embodiments, the low flavor protein composition has an average particle size of about 10 µm to about 40 µm in the largest dimension. In some embodiments, the low flavor protein composition has an average particle size of about 10 µm to about 30 µm in the largest dimension. In some embodiments, the low flavor protein composition has an average particle size of about 10 µm to about 20 µm in the largest dimension.

In some embodiments, the low flavor protein composition is in the form of an extrudate. In some embodiments, an extrudate is substantially in the form of granules.

In some embodiments, the granules have an average largest dimension of about 3 mm to about 5 mm. In some embodiments, less than about 20% (w/w) of the granules have a largest dimension less than 1 mm. In some embodiments, less than about 5% (w/w) of the granules have a largest dimension over 1 cm.

In some embodiments, the extrudate has a bulk density of about 0.25 to about 0.4 g/cm³. In some embodiments, the extrudate has a moisture content of about 5% to about 10%. In some embodiments, the extrudate has a protein content of about 65% to about 100% by dry weight. In some embodiments, the extrudate has a fat content of less than about 1.0%. In some embodiments, the extrudate has a sugar content of less than about 1%.

In some embodiments, the extrudate has a hydration ratio of about 2.5 to about 3 after about 60 minutes of hydration at room temperature. In some embodiments, the extrudate has a hydration time of less than about 30 minutes. In some embodiments, the extrudate has a pH of about 5.0 to about 7.5 when hydrated.

In some embodiments, the extrudate has a bite strength of about 2000 g to about 4000 g at a hydration ratio of about 3.

In some embodiments, the low flavor protein composition has a protein dispersibility index of at least about 5 (e.g., at least about 10 or at least about 15). In some embodiments, the low flavor protein composition has a sodium level up to about 1 %w/w (e.g., up to about 0.5, up to about 0.1, up to about 0.05, up to about 0.01, or up to about 0.005 %w/w).

In some embodiments, the low flavor protein composition has a solubility of at least 5% (e.g., at least 10%, at least 15%, at least 20%, at least 25%, or at least 30%) in an aqueous solution (e.g., water). In some embodiments, the aqueous solution has a pH of about 6.0 to about 8.0, of about 6.5 to about 7.5, of about 7.0 to about 8.0, of about 7.0, or of about 8.0. In some embodiments, the aqueous solution can include a buffer.

In some embodiments, the low flavor protein composition exhibits a temperature-dependent change in one or more mechanical properties (e.g., storage modulus, loss modulus, and/or viscosity) over a temperature range (e.g., heating from 25° C. to 95° C., heating from 40° C. to 95° C., heating from 60° C. to 95° C., or heating from 80° C. to 90° C.). In some embodiments, the temperature-dependent change is at least 5-fold (e.g., at least 10-fold, at least 100-fold, at least 500-fold, or at least 1,000-fold) in magnitude. In some embodiments, the temperature-dependent change is substantially irreversible (e.g., upon cooling over the same temperature range, the magnitude of the change is up to 25%, up to 20%, up to 15%, up to 10%, up to 5%, up to 1%, up to 0.5%, or up to 0.1% the magnitude of the change observed upon heating). In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 90° C. In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 95° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 90° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 95° C.

In some embodiments, the low flavor protein composition is a protein concentrate. In some embodiments, the low flavor protein composition is a protein isolate.

Also provided are a food product comprising any low flavor protein composition as described herein.

In another aspect, low color protein compositions are provided. Such low color protein compositions generally include at least 50% by dry weight of a plurality of plant proteins, fungal proteins, algal proteins, bacterial proteins, protozoan proteins, invertebrate proteins, or a combination thereof; wherein the plurality of plant proteins, fungal proteins, algal proteins, bacterial proteins, protozoan proteins, invertebrate proteins, or combination thereof are substantially aggregated, denatured, or both, and wherein the low color protein composition has a luminance of least 86 on a scale from 0 (black control value) to 100 (white control value), a chroma value of less than 14, or both.

In some embodiments, the low color protein composition has a luminance of at least 88 on a scale from 0 (black control value) to 100 (white control value). In some embodiments, the low color protein composition has a luminance of at least 90 on a scale from 0 (black control value) to 100 (white control value).

In some embodiments, the low color protein composition has a chroma value of less than 14. In some embodiments, the low color protein composition has a chroma value of less than 12. In some embodiments, the low color protein composition has a chroma value of less than 10. In some embodiments, the low color protein composition has a chroma value of less than 8. In some embodiments, the low color protein composition has a chroma value of less than 6.

In some embodiments, the low color protein composition comprises less than about 1.2% by dry weight lipids (e.g., less than about 1.0% or less than about 0.5% by dry weight lipids).

In some embodiments, the lipids comprise one or more of a fatty acid, a wax, a sterol, a monoglyceride, a diglyceride, a triglyceride, a sphingolipid or a phospholipid.

In some embodiments, the plurality of plant proteins, fungal proteins, algal proteins, bacterial proteins, protozoan proteins, invertebrate proteins, or a combination thereof is at least 90% by dry weight soy proteins.

In some embodiments, the low color protein composition further includes at least one of a preservative, an antioxidant, or a shelf life extender.

In some embodiments, the preservative, antioxidant, or shelf life extender comprises at least one of 4-hexylresorcinol, acetic acid, ascorbic acid, ascorbyl palmitate, ascorbyl stearate, benzoic acid, butylated hydroxyanisole (a mixture of 2-tertiarybutyl-4-hydroxyanisole and 3-tertiarybutyl-4-hydroxyanisole), butylated hydroxytoluene (3,5-ditertiarybutyl-4-hydroxytoluene), calcium ascorbate, calcium propionate, calcium sorbate, Carnobacterium divergens M35, Carnobacterium maltaromaticum cb1, carnosum 4010, citric acid, a citric acid ester of a monoglyceride or diglyceride, dimethyl dicarbonate, erythorbic acid, ethyl lauroyl arginate, gum guaiacum, iso-ascorbic acid, L-cysteine, L-cysteine hydrochloride, lecithin, lecithin citrate, Leuconostoc, methyl paraben, methyl-p-hydroxybenzoate, monoglyceride citrate, monoisopropyl citrate, natamycin, nisin, potassium acetate, potassium benzoate, potassium bisulfite, potassium diacetate, potassium lactate, potassium metabisulfite, potassium nitrate, potassium nitrite, potassium sorbate, propionic acid, propyl gallate, propyl paraben, propyl-p-hydroxy benzoate, sodium acetate, sodium ascorbate, sodium benzoate, sodium bisulfite, sodium diacetate, sodium dithionite, sodium erythorbate, sodium iso-ascorbate, sodium lactate, sodium metabisulfite, sodium nitrate, sodium nitrite, sodium propionate, sodium salt of methyl-p-hydroxy benzoic acid, sodium salt of propyl-p-hydroxy benzoic acid, sodium sorbate, sodium sulfite, sorbic acid, sulfurous acid, tartaric acid, tertiary butyl hydroquinone, or a tocopherol.

In some embodiments, the low color protein composition is in the form of a solution, suspension, or emulsion. In some embodiments, the low color protein composition is in the form of a solid or a powder.

In some embodiments, the low color protein composition has an average particle size of about 5 µm to about 40 µm in the largest dimension. In some embodiments, the low color protein composition has an average particle size of about 10 µm to about 40 µm in the largest dimension. In some embodiments, the low color protein composition has an average particle size of about 10 µm to about 30 µm in the largest dimension. In some embodiments, the low color protein composition has an average particle size of about 10 µm to about 20 µm in the largest dimension.

In some embodiments, the low color protein composition is in the form of an extrudate. In some embodiments, an extrudate is substantially in the form of granules.

In some embodiments, the granules have an average largest dimension of about 3 mm to about 5 mm. In some embodiments, less than about 20% (w/w) of the granules have a largest dimension less than 1 mm. In some embodiments, less than about 5% (w/w) of the granules have a largest dimension over 1 cm.

In some embodiments, the extrudate has a bulk density of about 0.25 to about 0.4 g/cm³. In some embodiments, the extrudate has a moisture content of about 5% to about 10%. In some embodiments, the extrudate has a protein content of about 65% to about 100% by dry weight. In some embodiments, the extrudate has a fat content of less than about 1.0%. In some embodiments, the extrudate has a sugar content of less than about 1%.

In some embodiments, the extrudate has a hydration ratio of about 2.5 to about 3 after about 60 minutes of hydration at room temperature. In some embodiments, the extrudate has a hydration time of less than about 30 minutes. In some embodiments, the extrudate has a pH of about 5.0 to about 7.5 when hydrated.

In some embodiments, the extrudate has a bite strength of about 2000 g to about 4000 g at a hydration ratio of about 3.

In some embodiments, the low color protein composition has a protein dispersibility index of at least about 5 (e.g., at least about 10 or at least about 15). In some embodiments, the low color protein composition has a sodium level up to about 1 %w/w (e.g., up to about 0.5, up to about 0.1, up to about 0.05, up to about 0.01, or up to about 0.005 %w/w).

In some embodiments, the low color protein composition has a solubility of at least 5% (e.g., at least 10%, at least 15%, at least 20%, at least 25%, or at least 30%) in an aqueous solution (e.g., water). In some embodiments, the aqueous solution has a pH of about 6.0 to about 8.0, of about 6.5 to about 7.5, of about 7.0 to about 8.0, of about 7.0, or of about 8.0. In some embodiments, the aqueous solution can include a buffer.

In some embodiments, the low color protein composition exhibits a temperature-dependent change in one or more mechanical properties (e.g., storage modulus, loss modulus, and/or viscosity) over a temperature range (e.g., heating from 25° C. to 95° C., heating from 40° C. to 95° C., heating from 60° C. to 95° C., or heating from 80° C. to 90° C.). In some embodiments, the temperature-dependent change is at least 5-fold (e.g., at least 10-fold, at least 100-fold, at least 500-fold, or at least 1,000-fold) in magnitude. In some embodiments, the temperature-dependent change is substantially irreversible (e.g., upon cooling over the same temperature range, the magnitude of the change is up to 25%, up to 20%, up to 15%, up to 10%, up to 5%, up to 1%, up to 0.5%, or up to 0.1% the magnitude of the change observed upon heating). In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 90° C. In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 95° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 90° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 95° C.

In some embodiments, the low color protein composition is a protein concentrate. In some embodiments, the low color protein composition is a protein isolate.

Also provided are a food product comprising any low color protein composition as described herein.

In still another aspect, protein concentrates are provided. Such protein concentrations generally include at least 50% by dry weight of a plurality of plant proteins, fungal proteins, algal proteins, bacterial proteins, protozoan proteins, invertebrate proteins, or a combination thereof; and at least 9% by dry weight of one or more insoluble carbohydrates, wherein the plurality of plant proteins, fungal proteins, algal proteins, bacterial proteins, protozoan proteins, invertebrate proteins, or combination thereof are substantially aggregated, denatured, or both.

In some embodiments, the protein concentrate has a luminance of at least 88 on a scale from 0 (black control value) to 100 (white control value). In some embodiments, the protein concentrate has a luminance of at least 90 on a scale from 0 (black control value) to 100 (white control value).

In some embodiments, the protein concentrate has a chroma value of less than 14. In some embodiments, the protein concentrate has a chroma value of less than 12. In some embodiments, the protein concentrate has a chroma value of less than 10. In some embodiments, the protein concentrate has a chroma value of less than 8. In some embodiments, the protein concentrate has a chroma value of less than 6.

In some embodiments, the protein concentrate comprises less than about 1.2% by dry weight lipids (e.g., less than about 1.0% or less than about 0.5% by dry weight lipids).

In some embodiments, the lipids comprise one or more of a fatty acid, a wax, a sterol, a monoglyceride, a diglyceride, a triglyceride, a sphingolipid, or a phospholipid.

In some embodiments, the plurality of plant proteins, fungal proteins, algal proteins, bacterial proteins, protozoan proteins, invertebrate proteins, or a combination thereof is at least 90% by dry weight soy proteins.

In some embodiments, the protein concentrate further includes at least one of a preservative, an antioxidant, or a shelf life extender.

In some embodiments, the preservative, antioxidant, or shelf life extender comprises at least one of 4-hexylresorcinol, acetic acid, ascorbic acid, ascorbyl palmitate, ascorbyl stearate, benzoic acid, butylated hydroxyanisole (a mixture of 2-tertiarybutyl-4-hydroxyanisole and 3-tertiarybutyl-4-hydroxyanisole), butylated hydroxytoluene (3,5-ditertiarybutyl-4-hydroxytoluene), calcium ascorbate, calcium propionate, calcium sorbate, Carnobacterium divergens M35, Carnobacterium maltaromaticum cb1, carnosum 4010, citric acid, a citric acid ester of a monoglyceride or diglyceride, dimethyl dicarbonate, erythorbic acid, ethyl lauroyl arginate, gum guaiacum, iso-ascorbic acid, L-cysteine, L-cysteine hydrochloride, lecithin, lecithin citrate, Leuconostoc, methyl paraben, methyl-p-hydroxybenzoate, monoglyceride citrate, monoisopropyl citrate, natamycin, nisin, potassium acetate, potassium benzoate, potassium bisulfite, potassium diacetate, potassium lactate, potassium metabisulfite, potassium nitrate, potassium nitrite, potassium sorbate, propionic acid, propyl gallate, propyl paraben, propyl-p-hydroxy benzoate, sodium acetate, sodium ascorbate, sodium benzoate, sodium bisulfite, sodium diacetate, sodium dithionite, sodium erythorbate, sodium iso-ascorbate, sodium lactate, sodium metabisulfite, sodium nitrate, sodium nitrite, sodium propionate, sodium salt of methyl-p-hydroxy benzoic acid, sodium salt of propyl-p-hydroxy benzoic acid, sodium sorbate, sodium sulfite, sorbic acid, sulfurous acid, tartaric acid, tertiary butyl hydroquinone, or a tocopherol.

In some embodiments, the protein concentrate is in the form of a solution, suspension, or emulsion. In some embodiments, the protein concentrate is in the form of a solid or a powder.

In some embodiments, the protein concentrate has an average particle size of about 5 µm to about 40 µm in the largest dimension. In some embodiments, the protein concentrate has an average particle size of about 10 µm to about 40 µm in the largest dimension. In some embodiments, the protein concentrate has an average particle size of about 10 µm to about 30 µm in the largest dimension. In some embodiments, the protein concentrate has an average particle size of about 10 µm to about 20 µm in the largest dimension.

In some embodiments, the protein concentrate is in the form of an extrudate. In some embodiments, an extrudate is substantially in the form of granules.

In some embodiments, the granules have an average largest dimension of about 3 mm to about 5 mm. In some embodiments, less than about 20% (w/w) of the granules have a largest dimension less than 1 mm. In some embodiments, less than about 5% (w/w) of the granules have a largest dimension over 1 cm.

In some embodiments, the extrudate has a bulk density of about 0.25 to about 0.4 g/cm³. In some embodiments, the extrudate has a moisture content of about 5% to about 10%. In some embodiments, the extrudate has a protein content of about 65% to about 100% by dry weight. In some embodiments, the extrudate has a fat content of less than about 1.0%. In some embodiments, the extrudate has a sugar content of less than about 1%.

In some embodiments, the extrudate has a hydration ratio of about 2.5 to about 3 after about 60 minutes of hydration at room temperature. In some embodiments, the extrudate has a hydration time of less than about 30 minutes. In some embodiments, the extrudate has a pH of about 5.0 to about 7.5 when hydrated.

In some embodiments, the extrudate has a bite strength of about 2000 g to about 4000 g at a hydration ratio of about 3.

In some embodiments, the protein concentrate has a protein dispersibility index of at least about 5 (e.g., at least about 10 or at least about 15). In some embodiments, the protein concentrate has a sodium level up to about 1 %w/w (e.g., up to about 0.5, up to about 0.1, up to about 0.05, up to about 0.01, or up to about 0.005 %w/w).

In some embodiments, the protein concentrate has a solubility of at least 5% (e.g., at least 10%, at least 15%, at least 20%, at least 25%, or at least 30%) in an aqueous solution (e.g., water). In some embodiments, the aqueous solution has a pH of about 6.0 to about 8.0, of about 6.5 to about 7.5, of about 7.0 to about 8.0, of about 7.0, or of about 8.0. In some embodiments, the aqueous solution can include a buffer.

In some embodiments, the protein concentrate exhibits a temperature-dependent change in one or more mechanical properties (e.g., storage modulus, loss modulus, and/or viscosity) over a temperature range (e.g., heating from 25° C. to 95° C., heating from 40° C. to 95° C., heating from 60° C. to 95° C., or heating from 80° C. to 90° C.). In some embodiments, the temperature-dependent change is at least 5-fold (e.g., at least 10-fold, at least 100-fold, at least 500-fold, or at least 1,000-fold) in magnitude. In some embodiments, the temperature-dependent change is substantially irreversible (e.g., upon cooling over the same temperature range, the magnitude of the change is up to 25%, up to 20%, up to 15%, up to 10%, up to 5%, up to 1%, up to 0.5%, or up to 0.1% the magnitude of the change observed upon heating). In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 90° C. In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 95° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 90° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 95° C.

Also provided are a food product comprising any protein concentrate as described herein.

In yet another aspect, protein isolates are provided. Such protein isolates generally include at least 50% by dry weight of a plurality of plant proteins, fungal proteins, algal proteins, bacterial proteins, protozoan proteins, invertebrate proteins, or a combination thereof; and less than 8% by dry weight of one or more insoluble carbohydrates, wherein the plurality of plant proteins, fungal proteins, algal proteins, bacterial proteins, protozoan proteins, invertebrate proteins, or combination thereof are substantially aggregated, denatured, or both.

In some embodiments, the protein isolate has a luminance of at least 88 on a scale from 0 (black control value) to 100 (white control value). In some embodiments, the protein isolate has a luminance of at least 90 on a scale from 0 (black control value) to 100 (white control value).

In some embodiments, the protein isolate has a chroma value of less than 14. In some embodiments, the protein isolate has a chroma value of less than 12. In some embodiments, the protein isolate has a chroma value of less than 10. In some embodiments, the protein isolate has a chroma value of less than 8. In some embodiments, the protein isolate has a chroma value of less than 6.

In some embodiments, the protein isolate comprises less than about 1.2% by dry weight lipids (e.g., less than about 1.0% or less than about 0.5% by dry weight lipids). In some embodiments, the lipids comprise one or more of a fatty acid, a wax, a sterol, a monoglyceride, a diglyceride, a triglyceride, a sphingolipid, or a phospholipid.

In some embodiments, the plurality of plant proteins, fungal proteins, algal proteins, bacterial proteins, protozoan proteins, invertebrate proteins, or a combination thereof is at least 90% by dry weight soy proteins.

In some embodiments, the protein isolate further includes at least one of a preservative, an antioxidant, or a shelf life extender.

In some embodiments, the preservative, antioxidant, or shelf life extender comprises at least one of 4-hexylresorcinol, acetic acid, ascorbic acid, ascorbyl palmitate, ascorbyl stearate, benzoic acid, butylated hydroxyanisole (a mixture of 2-tertiarybutyl-4-hydroxyanisole and 3-tertiarybutyl-4-hydroxyanisole), butylated hydroxytoluene (3,5-ditertiarybutyl-4-hydroxytoluene), calcium ascorbate, calcium propionate, calcium sorbate, Carnobacterium divergens M35, Carnobacterium maltaromaticum cb1, carnosum 4010, citric acid, a citric acid ester of a monoglyceride or diglyceride, dimethyl dicarbonate, erythorbic acid, ethyl lauroyl arginate, gum guaiacum, iso-ascorbic acid, L-cysteine, L-cysteine hydrochloride, lecithin, lecithin citrate, Leuconostoc, methyl paraben, methyl-p-hydroxybenzoate, monoglyceride citrate, monoisopropyl citrate, natamycin, nisin, potassium acetate, potassium benzoate, potassium bisulfite, potassium diacetate, potassium lactate, potassium metabisulfite, potassium nitrate, potassium nitrite, potassium sorbate, propionic acid, propyl gallate, propyl paraben, propyl-p-hydroxy benzoate, sodium acetate, sodium ascorbate, sodium benzoate, sodium bisulfite, sodium diacetate, sodium dithionite, sodium erythorbate, sodium iso-ascorbate, sodium lactate, sodium metabisulfite, sodium nitrate, sodium nitrite, sodium propionate, sodium salt of methyl-p-hydroxy benzoic acid, sodium salt of propyl-p-hydroxy benzoic acid, sodium sorbate, sodium sulfite, sorbic acid, sulfurous acid, tartaric acid, tertiary butyl hydroquinone, or a tocopherol.

In some embodiments, the protein isolate is in the form of a solution, suspension, or emulsion. In some embodiments, the protein isolate is in the form of a solid or a powder.

In some embodiments, the protein isolate has an average particle size of about 5 µm to about 40 µm in the largest dimension. In some embodiments, the protein isolate has an average particle size of about 10 µm to about 40 µm in the largest dimension.

In some embodiments, the protein isolate has an average particle size of about 10 µm to about 30 µm in the largest dimension. In some embodiments, the protein isolate has an average particle size of about 10 µm to about 20 µm in the largest dimension.

In some embodiments, the protein isolate is in the form of an extrudate. In some embodiments, an extrudate is substantially in the form of granules.

In some embodiments, the granules have an average largest dimension of about 3 mm to about 5 mm. In some embodiments, less than about 20% (w/w) of the granules have a largest dimension less than 1 mm. In some embodiments, less than about 5% (w/w) of the granules have a largest dimension over 1 cm.

In some embodiments, the extrudate has a bulk density of about 0.25 to about 0.4 g/cm³. In some embodiments, the extrudate has a moisture content of about 5% to about 10%. In some embodiments, the extrudate has a protein content of about 65% to about 100% by dry weight. In some embodiments, the extrudate has a fat content of less than about 1.0%. In some embodiments, the extrudate has a sugar content of less than about 1%.

In some embodiments, the extrudate has a hydration ratio of about 2.5 to about 3 after about 60 minutes of hydration at room temperature. In some embodiments, the extrudate has a hydration time of less than about 30 minutes. In some embodiments, the extrudate has a pH of about 5.0 to about 7.5 when hydrated.

In some embodiments, the extrudate has a bite strength of about 2000 g to about 4000 g at a hydration ratio of about 3.

In some embodiments, the protein isolate has a protein dispersibility index of at least about 5 (e.g., at least about 10 or at least about 15). In some embodiments, the protein isolate has a sodium level up to about 1 %w/w (e.g., up to about 0.5, up to about 0.1, up to about 0.05, up to about 0.01, or up to about 0.005%w/w).

In some embodiments, the protein isolate has a solubility of at least 5% (e.g., at least 10%, at least 15%, at least 20%, at least 25%, or at least 30%) in an aqueous solution (e.g., water). In some embodiments, the aqueous solution has a pH of about 6.0 to about 8.0, of about 6.5 to about 7.5, of about 7.0 to about 8.0, of about 7.0, or of about 8.0. In some embodiments, the aqueous solution can include a buffer.

In some embodiments, the protein isolate exhibits a temperature-dependent change in one or more mechanical properties (e.g., storage modulus, loss modulus, and/or viscosity) over a temperature range (e.g., heating from 25° C. to 95° C., heating from 40° C. to 95° C., heating from 60° C. to 95° C., or heating from 80° C. to 90° C.). In some embodiments, the temperature-dependent change is at least 5-fold (e.g., at least 10-fold, at least 100-fold, at least 500-fold, or at least 1,000-fold) in magnitude. In some embodiments, the temperature-dependent change is substantially irreversible (e.g., upon cooling over the same temperature range, the magnitude of the change is up to 25%, up to 20%, up to 15%, up to 10%, up to 5%, up to 1%, up to 0.5%, or up to 0.1% the magnitude of the change observed upon heating). In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 90° C. In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 95° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 90° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 95° C.

Also provided are a food product comprising any protein isolate as described herein.

In one aspect, low flavor protein compositions produced by the following methods are provided. Such methods generally include (a) adding an aqueous solution to a source protein composition to form a solution of solubilized protein; (b) optionally removing solids from the solution of solubilized protein; (c) adding an organic solvent to the solution of solubilized protein to form a solid phase and a liquid phase, and (d) separating the solid phase from the liquid phase to form a low flavor protein composition, wherein the low flavor protein composition comprises a plurality of plant proteins, fungal proteins, algal proteins, bacterial proteins, protozoan proteins, invertebrate proteins, or a combination thereof, and wherein the plurality of plant, fungal, algal, bacterial, protozoan, invertebrate proteins, or combination thereof are substantially aggregated, denatured, or both.

In some embodiments, step (a) is performed at a pH of about 6.0 to about 9.0. In some embodiments, step (a) is performed at a pH of about 7.5 to about 8.5. In some embodiments, step (a) is performed at a pH of about 7.0 to about 11.0 (e.g., about 7.0 to about 10.0, about 8.0 to about 10.0, about 8.0 to about 9.0, or about 8.0).

In some embodiments, step (b) comprises centrifugation, filtration, or a combination thereof.

In some embodiments, prior to step (c), the pH of the solution of solubilized protein is adjusted to about 4.0 to about 9.0. In some embodiments, prior to step (c), the pH of the solution of solubilized protein is adjusted to about 5.5 to about 7.5. In some embodiments, prior to step (c), the pH of the solution of solubilized protein is adjusted to about 6.0 to about 7.0. In some embodiments, prior to step (c), the pH of the solution of solubilized protein is adjusted to about 4.0 to about 7.0 (e.g., to about 4.0 to about 6.0, to about 4.5 to about 6.0, to about 4.5, or to about 6.0). In some embodiments, prior to step (c), the solution of solubilized protein is heated, for example, for about 10 seconds to about 30 minutes (e.g., about 10 seconds to about 20 minutes, about 10 seconds to about 30 seconds, about 10 seconds to about 1 minute, about 10 seconds to about 2 minutes, about 10 seconds to about 5 minutes, about 10 seconds to about 10 minutes, about 10 seconds to about 15 minutes, about 30 seconds to about 20 minutes, about 1 minute to about 30 minutes, about 1 minute to about 20 minutes, about 2 minutes to about 20 minutes, about 5 minutes to about 20 minutes, about 10 minutes to about 20 minutes, or about 15 minutes to about 20 minutes) at a temperature of about 70° C. to about 100° C. (e.g., about 80° C. to about 100° C., about 85° C. to about 100° C., about 85° C. to about 95° C., about 90° C. to about 100° C., about 85° C. to about 90° C., about 90° C. to about 95° C., or about 95° C. to about 100° C.). In some embodiments, prior to step (c), the organic solvent and/or the solution of solubilized protein are chilled, for example, to a temperature of about -20° C. to about 10° C. (e.g., about -20° C. to about 4° C.). In some embodiments, prior to step (c), the solution of solubilized protein is heated and then chilled.

In some embodiments, step (c) comprises adding an organic solvent. In some embodiments, step (c) comprises adding the organic solvent to a final concentration of about 5% to about 70% (v/v). In some embodiments, step (c) comprises adding the organic solvent to a final concentration of about 10% to about 50% (v/v). In some embodiments, step (c) comprises adding the organic solvent to a final concentration of about 20% to about 30% (v/v). In some embodiments, step (c) comprises adding the organic solvent to a final concentration of about 40% to about 90% (v/v) (e.g., to a final concentration of about 40% to about 70% (v/v), to a final concentration of about 40% to about 60% (v/v), or to a final concentration of about 45% to about 55% (v/v)).

In some embodiments, the pH is adjusted by adding an acid. In some embodiments, the acid is selected from the group consisting of hydrochloric acid, acetic acid, citric acid, tartaric acid, malic acid, folic acid, fumaric acid, and lactic acid. In some embodiments, the acid is hydrochloric acid.

In some embodiments, step (d) comprises centrifugation, filtration, or a combination thereof.

In some embodiments, the organic solvent is ethanol (e.g., at least 80%, at least 85%, at least 90%, at least 95%, at least 98%, at least 99%, at least 99.5%, or 100% ethanol). In some embodiments, the organic solvent is selected from the group consisting of ethanol, propanol, isopropyl alcohol, methanol, and acetone.

In some embodiments, the method further comprises (e) washing the low flavor protein composition with an organic wash solvent. In some embodiments, the method further comprises (e) washing the low flavor protein composition with an aqueous wash solvent. In some embodiments, the method further comprises (e) washing the low flavor protein composition with first an organic wash solvent and second an aqueous wash solvent, or vice versa.

In some embodiments, the organic wash solvent is ethanol (e.g., at least 80%, at least 85%, at least 90%, at least 95%, at least 98%, at least 99%, at least 99.5%, or 100% ethanol, or up to 20%, up to 15%, up to 10%, or up to 5% ethanol). In some embodiments, the organic wash solvent is selected from the group consisting of ethanol, propanol, isopropyl alcohol, methanol, and acetone.

In some embodiments, the organic wash solvent in step (e) is the same as the organic solvent in step (c).

In some embodiments, the aqueous wash solvent is water. In some embodiments, the aqueous wash solvent has a pH of about 6.0 to about 8.0, of about 6.5 to about 7.5, or of about 7.0. In some embodiments, the aqueous wash solvent can include a buffer.

In some embodiments, the method further includes drying the low flavor protein composition. In some embodiments, the drying includes spray drying, mat drying, freeze-drying, or oven drying.

In some embodiments, the source protein composition is at least 90% plant, algae, fungi, bacteria, protozoans, invertebrates, a part or derivative of any thereof, or a combination thereof on a dry weight basis. In some embodiments, source protein composition is at least 90% a defatted soy flour, a defatted pea flour, or a combination thereof on a dry weight basis. In some embodiments, the source protein composition is a soy protein composition, and the low flavor protein composition has an isoflavone content less than 90% of the isoflavone content of the source protein composition, on a dry weight basis. In some embodiments, the source protein composition is a soy protein composition, and the low flavor protein composition has an isoflavone content less than 70% of the isoflavone content of the source protein composition, on a dry weight basis. In some embodiments, the source protein composition is a soy protein composition, and the low flavor protein composition has an isoflavone content less than 50% of the isoflavone content of the source protein composition, on a dry weight basis. In some embodiments, the source protein composition is a soy protein composition, and the low flavor protein composition has an isoflavone content less than 30% of the isoflavone content of the source protein composition, on a dry weight basis. In some embodiments, the source protein composition is a soy protein composition, and the low flavor protein composition has an isoflavone content less than 10% of the isoflavone content of the source protein composition, on a dry weight basis.

In some embodiments, when cooked in water, a 1% (w/v) suspension of the low flavor protein composition by dry weight of the low flavor protein composition produces no more than 90% of the amount of one or more soy flavor compounds produced by cooking a 1% (w/v) suspension of the source protein composition (by dry weight of the source protein composition).

In some embodiments, when cooked in water, a 1% (w/v) suspension of the low flavor protein composition by dry weight of the low flavor protein composition produces no more than 70% of the amount of one or more soy flavor compounds produced by cooking a 1% (w/v) suspension of the source protein composition (by dry weight of the source protein composition).

In some embodiments, when cooked in water, a 1% (w/v) suspension of the low flavor protein composition by dry weight of the low flavor protein composition produces no more than 50% of the amount of one or more soy flavor compounds produced by cooking a 1% (w/v) suspension of the source protein composition (by dry weight of the source protein composition).

In some embodiments, when cooked in water, a 1% (w/v) suspension of the low flavor protein composition by dry weight of the low flavor protein composition produces no more than 30% of the amount of one or more soy flavor compounds produced by cooking a 1% (w/v) suspension of the source protein composition (by dry weight of the source protein composition).

In some embodiments, when cooked in water, a 1% (w/v) suspension of the low flavor protein composition by dry weight of the low flavor protein composition produces no more than 10% of the amount of one or more soy flavor compounds produced by cooking a 1% (w/v) suspension of the source protein composition (by dry weight of the source protein composition).

In some embodiments, when cooked in a flavor broth, a 1% (w/v) suspension of the low flavor protein composition by dry weight of the low flavor protein composition produces no more than 90% (e.g., no more than 80%, 70%, 60%, 50%, 40%, 30%, 20%, or 10%) of the amount of one or more soy flavor compounds produced by cooking a 1% (w/v) suspension of the source protein composition (by dry weight of the source protein composition).

In some embodiments, when cooked in a flavor broth, a 1% (w/v) suspension of the low flavor protein composition by dry weight of the low flavor protein composition produces at least 5% (e.g., at least 10%, 20%, 30%, 40%, 50%, 60%, 70%, 80%, 90%, 100%, or more) more of the amount of one or more volatile compounds in the meat volatile set produced by cooking a 1% (w/v) suspension of the source protein composition (by dry weight of the source protein composition).

In some embodiments, when cooked in a flavor broth, a 1% (w/v) suspension of the low flavor protein composition by dry weight of the protein composition produces at least 5% (e.g., at least 10%, 20%, 30%, 40%, 50%, 60%, 70%, 80%, 90%, 100%, or more) more of the amount of one or more volatile compounds in the meat volatile set produced by cooking a 1% (w/v) suspension of the source protein composition (by dry weight of the source protein composition).

In some embodiments, the one or more soy flavor compounds comprise at least one compound selected from the group consisting of hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal.

In some embodiments, the low flavor protein composition has a luminance of at least 88 on a scale from 0 (black control value) to 100 (white control value). In some embodiments, the low flavor protein composition has a luminance of at least 90 on a scale from 0 (black control value) to 100 (white control value).

In some embodiments, the low flavor protein composition has a chroma value of less than 14. In some embodiments, the low flavor protein composition has a chroma value of less than 12. In some embodiments, the low flavor protein composition has a chroma value of less than 10. In some embodiments, the low flavor protein composition has a chroma value of less than 8. In some embodiments, the low flavor protein composition has a chroma value of less than 6.

In some embodiments, the low flavor protein composition comprises less than about 1.2% by dry weight lipids (e.g., less than about 1.0% or less than about 0.5% by dry weight lipids). In some embodiments, the lipids comprise one or more of a fatty acid, a wax, a sterol, a monoglyceride, a diglyceride, a triglyceride, a sphingolipid, or a phospholipid.

In some embodiments, the plurality of plant proteins, fungal proteins, algal proteins, bacterial proteins, protozoan proteins, invertebrate proteins, or a combination thereof is at least 90% by dry weight soy proteins.

In some embodiments, low flavor protein composition further includes at least one of a preservative, an antioxidant, or a shelf life extender.

In some embodiments, the preservative, antioxidant, or shelf life extender comprises at least one of 4-hexylresorcinol, acetic acid, ascorbic acid, ascorbyl palmitate, ascorbyl stearate, benzoic acid, butylated hydroxyanisole (a mixture of 2-tertiarybutyl-4-hydroxyanisole and 3-tertiarybutyl-4-hydroxyanisole), butylated hydroxytoluene (3,5-ditertiarybutyl-4-hydroxytoluene), calcium ascorbate, calcium propionate, calcium sorbate, Carnobacterium divergens M35, Carnobacterium maltaromaticum cb1, carnosum 4010, citric acid, a citric acid ester of a monoglyceride or diglyceride, dimethyl dicarbonate, erythorbic acid, ethyl lauroyl arginate, gum guaiacum, iso-ascorbic acid, L-cysteine, L-cysteine hydrochloride, lecithin, lecithin citrate, Leuconostoc, methyl paraben, methyl-p-hydroxybenzoate, monoglyceride citrate, monoisopropyl citrate, natamycin, nisin, potassium acetate, potassium benzoate, potassium bisulfite, potassium diacetate, potassium lactate, potassium metabisulfite, potassium nitrate, potassium nitrite, potassium sorbate, propionic acid, propyl gallate, propyl paraben, propyl-p-hydroxy benzoate, sodium acetate, sodium ascorbate, sodium benzoate, sodium bisulfite, sodium diacetate, sodium dithionite, sodium erythorbate, sodium iso-ascorbate, sodium lactate, sodium metabisulfite, sodium nitrate, sodium nitrite, sodium propionate, sodium salt of methyl-p-hydroxy benzoic acid, sodium salt of propyl-p-hydroxy benzoic acid, sodium sorbate, sodium sulfite, sorbic acid, sulfurous acid, tartaric acid, tertiary butyl hydroquinone, or a tocopherol.

In some embodiments, the low flavor protein composition is in the form of a solution, suspension, or emulsion. In some embodiments, the low flavor protein composition is in the form of a solid or a powder.

In some embodiments, the low flavor protein composition has an average particle size of about 5 µm to about 40 µm in the largest dimension. In some embodiments, the low flavor protein composition has an average particle size of about 10 µm to about 40 µm in the largest dimension. In some embodiments, the low flavor protein composition has an average particle size of about 10 µm to about 30 µm in the largest dimension. In some embodiments, the low flavor protein composition has an average particle size of about 10 µm to about 20 µm in the largest dimension.

In some embodiments, the low flavor protein composition is in the form of an extrudate. In some embodiments, extrudate is substantially in the form of granules.

In some embodiments, the granules have an average largest dimension of about 3 mm to about 5 mm. In some embodiments, less than about 20% (w/w) of the granules have a largest dimension less than 1 mm. In some embodiments, less than about 5% (w/w) of the granules have a largest dimension over 1 cm.

In some embodiments, the extrudate has a bulk density of about 0.25 to about 0.4 g/cm³. In some embodiments, the extrudate has a moisture content of about 5% to about 10%. In some embodiments, the extrudate has a protein content of about 65% to about 100% by dry weight. In some embodiments, the extrudate has a fat content of less than about 1.0%. In some embodiments, the extrudate has a sugar content of less than about 1%.

In some embodiments, the extrudate has a hydration ratio of about 2.5 to about 3 after about 60 minutes of hydration at room temperature. In some embodiments, the extrudate has a hydration time of less than about 30 minutes. In some embodiments, the extrudate has a pH of about 5.0 to about 7.5 when hydrated.

In some embodiments, the extrudate has a bite strength of about 2000 g to about 4000 g at a hydration ratio of about 3.

In some embodiments, the low flavor protein composition has a protein dispersibility index of at least about 5 (e.g., at least about 10 or at least about 15). In some embodiments, the low flavor protein composition has a sodium level up to about 1 %w/w (e.g., up to about 0.5, up to about 0.1, up to about 0.05, up to about 0.01, or up to about 0.005 %w/w).

In some embodiments, the low flavor protein composition has a solubility of at least 5% (e.g., at least 10%, at least 15%, at least 20%, at least 25%, or at least 30%) in an aqueous solution (e.g., water). In some embodiments, the aqueous solution has a pH of about 6.0 to about 8.0, of about 6.5 to about 7.5, of about 7.0 to about 8.0, of about 7.0, or of about 8.0. In some embodiments, the aqueous solution can include a buffer.

In some embodiments, the low flavor protein composition exhibits a temperature-dependent change in one or more mechanical properties (e.g., storage modulus, loss modulus, and/or viscosity) over a temperature range (e.g., heating from 25° C. to 95° C., heating from 40° C. to 95° C., heating from 60° C. to 95° C., or heating from 80° C. to 90° C.). In some embodiments, the temperature-dependent change is at least 5-fold (e.g., at least 10-fold, at least 100-fold, at least 500-fold, or at least 1,000-fold) in magnitude. In some embodiments, the temperature-dependent change is substantially irreversible (e.g., upon cooling over the same temperature range, the magnitude of the change is up to 25%, up to 20%, up to 15%, up to 10%, up to 5%, up to 1%, up to 0.5%, or up to 0.1% the magnitude of the change observed upon heating). In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 90° C. In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 95° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 90° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 95° C.

In some embodiments, the low flavor protein composition is a protein concentrate. In some embodiments, the low flavor protein composition is a protein isolate.

A food product including the low flavor protein composition as described herein.

In another aspect, provided herein is a protein composition including at least 50% by dry weight of a plurality of plant proteins, fungal proteins, algal proteins, bacterial proteins, protozoan proteins, invertebrate proteins, or a combination thereof, and less than 1.2% by dry weight fat, and wherein the plurality of plant proteins, fungal proteins, algal proteins, bacterial proteins, protozoan proteins, invertebrate proteins, or combination thereof are substantially aggregated, denatured, or both.

Implementations can include one or more of the following features. The protein composition can have a luminance of at least 86 on a scale from 0 (black control value) to 100 (white control value). The protein composition can have a luminance of at least 90 on a scale from 0 (black control value) to 100 (white control value). The protein composition can have a chroma value of less than 14. The protein composition can have a chroma value of less than 12. The protein composition can have a chroma value of less than 10. The composition can have a chroma value of less than 8. The protein composition can have a chroma value of less than 6. The protein composition can include less than about 0.5% by dry weight lipids. The lipids can include one or more of a fatty acid, a wax, a sterol, a monoglyceride, a diglyceride, a triglyceride, a sphingolipid or a phospholipid. The plurality of plant proteins, fungal proteins, algal proteins, bacterial proteins, protozoan proteins, invertebrate proteins, or a combination thereof can be at least 90% by dry weight soy proteins. The composition can further include at least one of a preservative, an antioxidant, or a shelf life extender. The protein composition can be in the form of a solution, suspension, or emulsion. The protein composition can be in the form of a solid or a powder. The protein composition can have an average particle size of about 5 µm to about 40 µm in the largest dimension. The protein composition can have an average particle size of about 10 µm to about 40 µm in the largest dimension. The protein composition can have an average particle size of about 10 µm to about 30 µm in the largest dimension. The protein composition can have an average particle size of about 10 µm to about 20 µm in the largest dimension. The protein composition is in the form of an extrudate. The extrudate can be substantially in the form of granules. The granules can have an average largest dimension of about 3 mm to about 5 mm. Less than about 20% (w/w) of the granules can have a largest dimension less than 1 mm. Less than about 5% (w/w) of the granules can have a largest dimension over 1 cm. The extrudate can have a bulk density of about 0.25 to about 0.4 g/cm³. The extrudate can have a moisture content of about 5% to about 10%. The extrudate can have a protein content of about 65% to about 100% by dry weight. The extrudate can have a fat content of less than about 1.0%. The extrudate can have a sugar content of less than about 1%. The extrudate can have a hydration ratio of about 2.5 to about 3 after about 60 minutes of hydration at room temperature. The extrudate can have a hydration time of less than about 30 minutes. The extrudate can have a pH of about 5.0 to about 7.5 when hydrated. The extrudate can have a bite strength of about 2000 g to about 4000 g at a hydration ratio of about 3. The protein composition can be a protein concentrate. The protein composition can be a protein isolate. Also provided herein are food products comprising any of the protein compositions provided herein.

In another aspect, methods for producing a low flavor protein composition are provided. Such methods typically include (a) adding an aqueous solution to a source protein composition to form a solution of solubilized protein; (b) optionally removing solids from the solution of solubilized protein; (c) adding an organic solvent to the solution of solubilized protein to form a solid phase and a liquid phase, and (d) separating the solid phase from the liquid phase to form a low flavor protein composition, wherein the low flavor protein composition can include a plurality of plant proteins, fungal proteins, algal proteins, bacterial proteins, protozoan proteins, invertebrate proteins, or a combination thereof.

Implementations can include one or more of the following features. Step (a) can be performed at a pH of about 6.0 to about 9.0. Step (a) can be performed at a pH of about 7.5 to about 8.5. Step (a) can be performed at a pH of about 7.0 to about 11.0 (e.g., about 7.0 to about 10.0, about 8.0 to about 10.0, about 8.0 to about 9.0, or about 8.0). Step (b) can include centrifugation, filtration, or a combination thereof. Prior to step (c), the pH of the solution of solubilized protein can be adjusted to about 4.0 to about 9.0. Prior to step (c), the pH of the solution of solubilized protein can be adjusted to about 5.5 to about 7.5. Prior to step (c), the pH of the solution of solubilized protein can be adjusted to about 6.0 to about 7.0. Prior to step (c), the pH of the solution of solubilized protein can be adjusted to about 4.0 to about 7.0 (e.g., to about 4.0 to about 6.0, to about 4.5 to about 6.0, to about 4.5, or to about 6.0). In some embodiments, prior to step (c), the solution of solubilized protein is heated, for example, for about 10 seconds to about 30 minutes (e.g., about 10 seconds to about 20 minutes, about 10 seconds to about 30 seconds, about 10 seconds to about 1 minute, about 10 seconds to about 2 minutes, about 10 seconds to about 5 minutes, about 10 seconds to about 10 minutes, about 10 seconds to about 15 minutes, about 30 seconds to about 20 minutes, about 1 minute to about 30 minutes, about 1 minute to about 20 minutes, about 2 minutes to about 20 minutes, about 5 minutes to about 20 minutes, about 10 minutes to about 20 minutes, or about 15 minutes to about 20 minutes) at a temperature of about 70° C. to about 100° C. (e.g., about 80° C. to about 100° C., about 85° C. to about 100° C., about 85° C. to about 95° C., about 90° C. to about 100° C., about 85° C. to about 90° C., about 90° C. to about 95° C., or about 95° C. to about 100° C.). In some embodiments, prior to step (c), the organic solvent and/or the solution of solubilized protein are chilled, for example, to a temperature of about -20° C. to about 10° C. (e.g., about -20° C. to about 4° C.). In some embodiments, prior to step (c), the solution of solubilized protein is heated and then chilled. Step (c) can comprise adding an organic solvent. Step (c) can include adding the organic solvent to a final concentration of about 5% to about 70% (v/v). Step (c) can include adding the organic solvent to a final concentration of about 10% to about 50% (v/v). Step (c) can include adding the organic solvent to a final concentration of about 20% to about 30% (v/v). Step (c) can include adding the organic solvent to a final concentration of about 40% to about 90% (v/v) (e.g., to a final concentration of about 40% to about 70% (v/v), to a final concentration of about 40% to about 60% (v/v), or to a final concentration of about 45% to about 55% (v/v)). The pH can be adjusted by adding an acid. In some embodiments, the acid is selected from the group consisting of hydrochloric acid, acetic acid, citric acid, tartaric acid, malic acid, folic acid, fumaric acid, and lactic acid. In some embodiments, the acid is hydrochloric acid. Step (d) can include centrifugation, filtration, or a combination thereof. The organic solvent can be ethanol (e.g., at least 80%, at least 85%, at least 90%, at least 95%, at least 98%, at least 99%, at least 99.5%, or 100% ethanol). In some embodiments, the organic solvent is selected from the group consisting of ethanol, propanol, isopropyl alcohol, methanol, and acetone. The method further can further include (e) washing the low flavor protein composition with an organic wash solvent. The method further can include (e) washing the low flavor protein composition with an aqueous wash solvent. The method further can include (e) washing the low flavor protein composition with first an organic wash solvent and second an aqueous wash solvent, or vice versa. The organic wash solvent can be ethanol (e.g., at least 80%, at least 85%, at least 90%, at least 95%, at least 98%, at least 99%, at least 99.5%, or 100% ethanol, or up to 20%, up to 15%, up to 10%, or up to 5% ethanol). In some embodiments, the organic wash solvent is selected from the group consisting of ethanol, propanol, isopropyl alcohol, methanol, and acetone. The organic wash solvent in step (e) can be the same as the organic solvent in step (c). The aqueous wash solvent can be water. In some embodiments, the aqueous wash solvent has a pH of about 6.0 to about 8.0, of about 6.5 to about 7.5, or of about 7.0. In some embodiments, the aqueous wash solvent can include a buffer. The method can further comprise drying the low flavor protein composition. Drying can include spray drying, mat drying, freeze-drying, or oven drying. The source protein composition can be at least 90% plant, algae, fungi, bacteria, protozoans, invertebrates, a part or derivative of any thereof, or a combination thereof on a dry weight basis. The source protein composition can be at least 90% a defatted soy flour, a defatted pea flour, or a combination thereof on a dry weight basis. The source protein composition can be a soy protein composition, and the low flavor protein composition can have an isoflavone content less than 90% of the isoflavone content of the source protein composition, on a dry weight basis. The source protein composition can be a soy protein composition, and the low flavor protein composition can have an isoflavone content less than 70% of the isoflavone content of the source protein composition, on a dry weight basis. The source protein composition can be a soy protein composition, and the low flavor protein composition can have an isoflavone content less than 50% of the isoflavone content of the source protein composition, on a dry weight basis. The source protein composition can be a soy protein composition, and the low flavor protein composition can have an isoflavone content less than 30% of the isoflavone content of the source protein composition, on a dry weight basis. The source protein composition can be a soy protein composition, and the low flavor protein composition can have an isoflavone content less than 10% of the isoflavone content of the source protein composition, on a dry weight basis. When cooked in water, a 1% (w/v) suspension of the low flavor protein composition by dry weight of the low flavor protein composition can produce no more than 90% of the amount of one or more soy flavor compounds produced by cooking a 1% (w/v) suspension of the source protein composition (by dry weight of the source protein composition). When cooked in water, a 1% (w/v) suspension of the low flavor protein composition by dry weight of the low flavor protein composition can produce no more than 70% of the amount of one or more soy flavor compounds produced by cooking a 1% (w/v) suspension of the source protein composition (by dry weight of the source protein composition). When cooked in water, a 1% (w/v) suspension of the low flavor protein composition by dry weight of the low flavor protein composition can produce no more than 50% of the amount of one or more soy flavor compounds produced by cooking a 1% (w/v) suspension of the source protein composition (by dry weight of the source protein composition). When cooked in water, a 1% (w/v) suspension of the low flavor protein composition by dry weight of the low flavor protein composition can produce no more than 30% of the amount of one or more soy flavor compounds produced by cooking a 1% (w/v) suspension of the source protein composition (by dry weight of the source protein composition). When cooked in water, a 1% (w/v) suspension of the low flavor protein composition by dry weight of the low flavor protein composition can produce no more than 10% of the amount of one or more soy flavor compounds produced by cooking a 1% (w/v) suspension of the source protein composition (by dry weight of the source protein composition). When cooked in a flavor broth, a 1% (w/v) suspension of the low flavor protein composition by dry weight of the low flavor protein composition can produce no more than 90% (e.g., no more than 80%, 70%, 60%, 50%, 40%, 30%, 20%, or 10%) of the amount of one or more soy flavor compounds produced by cooking a 1% (w/v) suspension of the source protein composition (by dry weight of the source protein composition). In some embodiments, the protein composition produces no more than 90% (e.g., no more than 70%, 50%, 30%, or 10%) of the amount of one or more volatile compounds in a set of volatile compounds produced by the source protein composition by solvent-assisted flavor extraction (SAFE). The one or more soy flavor compounds comprise at least one compound selected from the group consisting of hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal. The low flavor protein composition can have a luminance of at least 88 on a scale from 0 (black control value) to 100 (white control value). When cooked in a flavor broth, a 1% (w/v) suspension of the low flavor protein composition by dry weight of the low flavor protein composition can produce at least 5% (e.g., at least 10%, 20%, 30%, 40%, 50%, 60%, 70%, 80%, 90%, 100%, or more) more of the amount of one or more volatile compounds in the meat volatile set produced by cooking a 1% (w/v) suspension of the source protein composition (by dry weight of the source protein composition). The low flavor protein composition can have a luminance of at least 90 on a scale from 0 (black control value) to 100 (white control value). The low flavor protein composition can have a chroma value of less than 14. The low flavor protein composition can have a chroma value of less than 12. The low flavor protein composition can have a chroma value of less than 10. The low flavor protein composition can have a chroma value of less than 8. The low flavor protein composition can have a chroma value of less than 6. The low flavor protein composition can include less than about 1.2% by dry weight lipids (e.g., less than about 1.0% or less than about 0.5% by dry weight lipids). The lipids can include one or more of a fatty acid, a wax, a sterol, a monoglyceride, a diglyceride, a triglyceride, a sphingolipid, or a phospholipid. The plurality of plant proteins, fungal proteins, algal proteins, bacterial proteins, protozoan proteins, invertebrate proteins, or a combination thereof can be at least 90% by dry weight soy proteins. The low flavor protein composition can include at least one of a preservative, an antioxidant, or a shelf life extender. The low flavor protein composition can be in the form of a solution, suspension, or emulsion. The low flavor protein composition can be in the form of a solid or a powder. The low flavor protein composition can have an average particle size of about 5 µm to about 40 µm in the largest dimension. The low flavor protein composition can have an average particle size of about 5 µm to about 40 µm in the largest dimension. The low flavor protein composition can have an average particle size of about 10 µm to about 30 µm in the largest dimension. The low flavor protein composition can have an average particle size of about 10 µm to about 20 µm in the largest dimension. The low flavor protein composition can be in the form of an extrudate. The extrudate can be substantially in the form of granules. The granules can have an average largest dimension of about 3 mm to about 5 mm. Less than about 20% (w/w) of the granules can have a largest dimension less than 1 mm. Less than about 5% (w/w) of the granules can have a largest dimension over 1 cm. The extrudate can have a bulk density of about 0.25 to about 0.4 g/cm³. The extrudate can have a moisture content of about 5% to about 10%. The extrudate can have a protein content of about 65% to about 100% by dry weight. The extrudate can have a fat content of less than about 1.0%. The extrudate can have a sugar content of less than about 1%. The extrudate can have a hydration ratio of about 2.5 to about 3 after about 60 minutes of hydration at room temperature. The extrudate can have a hydration time of less than about 30 minutes. The extrudate can have a pH of about 5.0 to about 7.5 when hydrated. The extrudate can have a bite strength of about 2000 g to about 4000 g at a hydration ratio of about 3. In some embodiments, the low flavor protein composition has a protein dispersibility index of at least about 5 (e.g., at least about 10 or at least about 15). In some embodiments, the low flavor protein composition has a sodium level up to about 1 %w/w (e.g., up to about 0.5, up to about 0.1, up to about 0.05, up to about 0.01, or up to about 0.005 %w/w). In some embodiments, the low flavor protein composition has a solubility of at least 5% (e.g., at least 10%, at least 15%, at least 20%, at least 25%, or at least 30%) in an aqueous solution (e.g., water). In some embodiments, the aqueous solution has a pH of about 6.0 to about 8.0, of about 6.5 to about 7.5, of about 7.0 to about 8.0, of about 7.0, or of about 8.0. In some embodiments, the aqueous solution can include a buffer. In some embodiments, the low flavor protein composition exhibits a temperature-dependent change in one or more mechanical properties (e.g., storage modulus, loss modulus, and/or viscosity) over a temperature range (e.g., heating from 25° C. to 95° C., heating from 40° C. to 95° C., heating from 60° C. to 95° C., or heating from 80° C. to 90° C.). In some embodiments, the temperature-dependent change is at least 5-fold (e.g., at least 10-fold, at least 100-fold, at least 500-fold, or at least 1,000-fold) in magnitude. In some embodiments, the temperature-dependent change is substantially irreversible (e.g., upon cooling over the same temperature range, the magnitude of the change is up to 25%, up to 20%, up to 15%, up to 10%, up to 5%, up to 1%, up to 0.5%, or up to 0.1% the magnitude of the change observed upon heating). In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 90° C. In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa·s, or at least 10,000 Pa) at 95° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 90° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 95° C. The low flavor protein composition can be a protein concentrate. The low flavor protein composition can be a protein isolate.

Also provided herein is a food product comprising a low flavor protein composition produced by any of the methods described herein.

In still another aspect, methods for making a detoxified protein composition are provided. Such methods generally include (a) adding an aqueous solution to a source protein composition to form a solution of solubilized protein; (b) optionally removing solids from the solution of solubilized protein; (c) adding an organic solvent to the solution of solubilized protein to form a solid phase and a liquid phase, and (d) separating the solid phase from the liquid phase to form a detoxified protein composition, wherein the detoxified protein composition can include a plurality of plant proteins, fungal proteins, algal proteins, bacterial proteins, protozoan proteins, invertebrate proteins, wherein the source protein composition can be not suitable for human consumption.

Implementations can include one or more of the following features. The source protein composition can include one or more toxins in an amount sufficient to harm a human being. The source protein composition can be a cottonwood source protein composition. The source protein composition can include gossypol in an amount of more than 450 ppm. The detoxified protein composition can include gossypol in an amount of less than 450 ppm. The detoxified protein composition can include gossypol in an amount of less than 300 ppm. The detoxified protein composition can include gossypol in an amount of less than 100 ppm. The detoxified protein composition can include gossypol in an amount of less than 10 ppm. In some embodiments, a detoxified protein composition as described herein can include one or more toxins in an amount smaller than the amount in the source protein composition. In some cases, a detoxified protein composition can have a toxin content of less than about 90% (e.g., less than about 70%, 50%, 30%, or 10%) of the toxin content of the source protein composition. Non-limiting examples of toxins include gossypol (for example, in cottonwood), vicine or convicine (for example, in faba beans), cyanogenic glycosides (for example, in cassava or bamboo), glucosinolates (for example, in cruciferous vegetables), and glycoalkaloids (for example, in potato and bittersweet nightshade). Step (a) can be performed at a pH of about 6.0 to about 9.0. Step (a) can be performed at a pH of about 7.5 to about 8.5. Step (a) can be performed at a pH of about 7.0 to about 11.0 (e.g., about 7.0 to about 10.0, about 8.0 to about 10.0, about 8.0 to about 9.0, or about 8.0). Step (b) can include centrifugation, filtration, or a combination thereof. Prior to step (c), the pH of the solution of solubilized protein can be adjusted to about 4.0 to about 9.0. Prior to step (c), the pH of the solution of solubilized protein can be adjusted to about 5.5 to about 7.5. Prior to step (c), the pH of the solution of solubilized protein can be adjusted to about 6.0 to about 7.0. Prior to step (c), the pH of the solution of solubilized protein can be adjusted to about 4.0 to about 7.0 (e.g., to about 4.0 to about 6.0, to about 4.5 to about 6.0, to about 4.5, or to about 6.0). In some embodiments, prior to step (c), the solution of solubilized protein is heated, for example, for about 10 seconds to about 30 minutes (e.g., about 10 seconds to about 20 minutes, about 10 seconds to about 30 seconds, about 10 seconds to about 1 minute, about 10 seconds to about 2 minutes, about 10 seconds to about 5 minutes, about 10 seconds to about 10 minutes, about 10 seconds to about 15 minutes, about 30 seconds to about 20 minutes, about 1 minute to about 30 minutes, about 1 minute to about 20 minutes, about 2 minutes to about 20 minutes, about 5 minutes to about 20 minutes, about 10 minutes to about 20 minutes, or about 15 minutes to about 20 minutes) at a temperature of about 70° C. to about 100° C. (e.g., about 80° C. to about 100° C., about 85° C. to about 100° C., about 85° C. to about 95° C., about 90° C. to about 100° C., about 85° C. to about 90° C., about 90° C. to about 95° C., or about 95° C. to about 100° C.). In some embodiments, prior to step (c), the organic solvent and/or the solution of solubilized protein are chilled, for example, to a temperature of about -20° C. to about 10° C. (e.g., about -20° C. to about 4° C.). In some embodiments, prior to step (c), the solution of solubilized protein is heated and then chilled. Step (c) can comprise adding an organic solvent. Step (c) can include adding the organic solvent to a final concentration of about 5% to about 70% (v/v). Step (c) can include adding the organic solvent to a final concentration of about 10% to about 50% (v/v). Step (c) can include adding the organic solvent to a final concentration of about 20% to about 30% (v/v). Step (c) can include adding the organic solvent to a final concentration of about 40% to about 90% (v/v) (e.g., to a final concentration of about 40% to about 70% (v/v), to a final concentration of about 40% to about 60% (v/v), or to a final concentration of about 45% to about 55% (v/v)). The pH can be adjusted by adding an acid. In some embodiments, the acid is selected from the group consisting of hydrochloric acid, acetic acid, citric acid, tartaric acid, malic acid, folic acid, fumaric acid, and lactic acid. In some embodiments, the acid is hydrochloric acid. Step (d) can include centrifugation, filtration, or a combination thereof. The organic solvent can be ethanol (e.g., at least 80%, at least 85%, at least 90%, at least 95%, at least 98%, at least 99%, at least 99.5%, or 100% ethanol). In some embodiments, the organic solvent is selected from the group consisting of ethanol, propanol, isopropyl alcohol, methanol, and acetone. The method further can include (e) washing the low flavor protein composition with an organic wash solvent. The method further can include (e) washing the low flavor protein composition with an aqueous wash solvent. The method further can include (e) washing the low flavor protein composition with first an organic wash solvent and second an aqueous wash solvent, or vice versa. The organic wash solvent can be ethanol (e.g., at least 80%, at least 85%, at least 90%, at least 95%, at least 98%, at least 99%, at least 99.5%, or 100% ethanol, or up to 20%, up to 15%, up to 10%, or up to 5% ethanol). In some embodiments, the organic wash solvent is selected from the group consisting of ethanol, propanol, isopropyl alcohol, methanol, and acetone. The organic wash solvent in step (e) can be the same as the organic solvent in step (c). The aqueous wash solvent can be water. In some embodiments, the aqueous wash solvent has a pH of about 6.0 to about 8.0, of about 6.5 to about 7.5, or of about 7.0. In some embodiments, the aqueous wash solvent can include a buffer. The method can further comprise drying the detoxified protein composition. Drying can include spray drying, mat drying, freeze-drying, or oven drying. The source protein composition can be at least 90% plant, algae, fungi, bacteria, protozoans, invertebrates, a part or derivative of any thereof, or a combination thereof on a dry weight basis.

In yet another aspect, methods of extracting small molecules from a protein source composition are provided. Such methods generally include (a) adding an aqueous solution to a source protein composition to form a solution of solubilized protein; (b) optionally removing solids from the solution of solubilized protein; (c) adding an organic solvent to the solution of solubilized protein to form a solid phase and a liquid phase, and (d) separating the solid phase from the liquid phase to form a solution enriched in small molecules.

Implementations can include one or more of the following features. The source protein composition can be a soy source protein composition. The solution enriched in small molecules can include isoflavones. The solution enriched in small molecules can include isoflavones, pigments (e.g., chlorophylls, anthocyanins, carotenoids, and betalains), flavor compounds (e.g., soy flavor compounds), saponin, toxins (e.g., gossypol), natural products (e.g., plant natural products, pharmacologically active natural products), metabolites (e.g., primary and/or secondary metabolites), and/or phospholipids (e.g., lecithin). The small molecules can have molecular weights up to 900 daltons (e.g., up to 800, up to 700, up to 600, or up to 500 daltons). Step (a) can be performed at a pH of about 6.0 to about 9.0. Step (a) can be performed at a pH of about 7.5 to about 8.5. Step (a) can be performed at a pH of about 7.0 to about 11.0 (e.g., about 7.0 to about 10.0, about 8.0 to about 10.0, about 8.0 to about 9.0, or about 8.0). Step (b) can include centrifugation, filtration, or a combination thereof. Prior to step (c), the pH of the solution of solubilized protein can be adjusted to about 4.0 to about 9.0. Prior to step (c), the pH of the solution of solubilized protein can be adjusted to about 5.5 to about 7.5. Prior to step (c), the pH of the solution of solubilized protein can be adjusted to about 6.0 to about 7.0. Prior to step (c), the pH of the solution of solubilized protein can be adjusted to about 4.0 to about 7.0 (e.g., to about 4.0 to about 6.0, to about 4.5 to about 6.0, to about 4.5, or to about 6.0). In some embodiments, prior to step (c), the solution of solubilized protein is heated, for example, for about 10 seconds to about 30 minutes (e.g., about 10 seconds to about 20 minutes, about 10 seconds to about 30 seconds, about 10 seconds to about 1 minute, about 10 seconds to about 2 minutes, about 10 seconds to about 5 minutes, about 10 seconds to about 10 minutes, about 10 seconds to about 15 minutes, about 30 seconds to about 20 minutes, about 1 minute to about 30 minutes, about 1 minute to about 20 minutes, about 2 minutes to about 20 minutes, about 5 minutes to about 20 minutes, about 10 minutes to about 20 minutes, or about 15 minutes to about 20 minutes) at a temperature of about 70° C. to about 100° C. (e.g., about 80° C. to about 100° C., about 85° C. to about 100° C., about 85° C. to about 95° C., about 90° C. to about 100° C., about 85° C. to about 90° C., about 90° C. to about 95° C., or about 95° C. to about 100° C.). In some embodiments, prior to step (c), the organic solvent and/or the solution of solubilized protein are chilled, for example, to a temperature of about -20° C. to about 10° C. (e.g., about -20° C. to about 4° C.). In some embodiments, prior to step (c), the solution of solubilized protein is heated and then chilled. Step (c) can comprise adding an organic solvent. Step (c) can include adding the organic solvent to a final concentration of about 5% to about 70% (v/v). Step (c) can include adding the organic solvent to a final concentration of about 10% to about 50% (v/v). Step (c) can include adding the organic solvent to a final concentration of about 20% to about 30% (v/v). Step (c) can include adding the organic solvent to a final concentration of about 40% to about 90% (v/v) (e.g., to a final concentration of about 40% to about 70% (v/v), to a final concentration of about 40% to about 60% (v/v), or to a final concentration of about 45% to about 55% (v/v)). The pH can be adjusted by adding an acid. In some embodiments, the acid is selected from the group consisting of hydrochloric acid, acetic acid, citric acid, tartaric acid, malic acid, folic acid, fumaric acid, and lactic acid. In some embodiments, the acid is hydrochloric acid. Step (d) can include centrifugation, filtration, or a combination thereof. The organic solvent can be ethanol (e.g., at least 80%, at least 85%, at least 90%, at least 95%, at least 98%, at least 99%, at least 99.5%, or 100% ethanol). In some embodiments, the organic solvent is selected from the group consisting of ethanol, propanol, isopropyl alcohol, methanol, and acetone. The method further can include (e) washing the low flavor protein composition with an organic wash solvent. The method further can include (e) washing the low flavor protein composition with an aqueous wash solvent. The method further can include (e) washing the low flavor protein composition with first an organic wash solvent and second an aqueous wash solvent, or vice versa. The organic wash solvent can be ethanol (e.g., at least 80%, at least 85%, at least 90%, at least 95%, at least 98%, at least 99%, at least 99.5%, or 100% ethanol, or up to 20%, up to 15%, up to 10%, or up to 5% ethanol). In some embodiments, the organic wash solvent is selected from the group consisting of ethanol, propanol, isopropyl alcohol, methanol, and acetone. The organic wash solvent in step (e) can be the same as the organic solvent in step (c). The aqueous wash solvent can be water. In some embodiments, the aqueous wash solvent has a pH of about 6.0 to about 8.0, of about 6.5 to about 7.5, or of about 7.0. In some embodiments, the aqueous wash solvent can include a buffer. The method can further include drying the low flavor protein composition. Drying can include spray drying, mat drying, freeze-drying, or oven drying. The source protein composition can be at least 90% plant, algae, fungi, bacteria, protozoans, invertebrates, a part or derivative of any thereof, or a combination thereof on a dry weight basis.

In yet another aspect, food products are provided. Such food products optionally include a fat; optionally one or more flavor precursor compounds; and at least 10% by dry weight of a low flavor protein composition, the low flavor protein composition comprising at least 50% by dry weight of a plurality of plant proteins, fungal proteins, algal proteins, bacterial proteins, protozoan proteins, invertebrate proteins, or a combination thereof, and wherein the plurality of plant, fungal, algal, bacterial, protozoan, invertebrate proteins, or combination thereof are substantially aggregated, denatured, or both.

Implementations can include one or more of the following features. The food product can be a plant-based food product. The food product can be an algae-based food product. The food product can be a fungus-based food product. The food product can be an invertebrate-based food product. The fat can include at least one fat selected from the group consisting of corn oil, olive oil, soy oil, peanut oil, walnut oil, almond oil, sesame oil, cottonseed oil, rapeseed oil, canola oil, safflower oil, sunflower oil, flax seed oil, palm oil, palm kernel oil, coconut oil, babassu oil, shea butter, mango butter, cocoa butter, wheat germ oil, rice bran oil, and combinations thereof. The one or more flavor precursors can comprise at least one compound selected from the group consisting of glucose, ribose, cysteine, a cysteine derivative, thiamine, alanine, methionine, lysine, a lysine derivative, glutamic acid, a glutamic acid derivative, IMP, GMP, lactic acid, maltodextrin, creatine, alanine, arginine, asparagine, aspartate, glutamic acid, glutamine, glycine, histidine, isoleucine, leucine, methionine, phenylalanine, proline, threonine, tryptophan, tyrosine, valine, linoleic acid, and mixtures thereof. Suitable flavor precursors can include sugars, sugar alcohols, sugar derivatives, oils (e.g., vegetable oils), free fatty acids, alpha-hydroxy acids, dicarboxylic acids, amino acids and derivatives thereof, nucleosides, nucleotides, vitamins, peptides, protein hydrolysates, extracts, phospholipids, lecithin, and organic molecules. The food product can be a meat analog. The food product can be in the form of ground meat, a sausage, or a cut of meat. The food product can be a dairy analog (e.g., milk, fermented milk, yogurt, cream, butter, cheese, custard, ice cream, gelato, or frozen yogurt). The food product can contain no animal products. The fat can be present in the food product in an amount of about 5% to about 80% by dry weight of the food product. The fat can be present in the food product in an amount of about 10% to about 30% by dry weight of the food product. The food product can contain no fat. The food product can further include about 0.01% to about 5% by dry weight of a heme-containing protein. The food product can be a beverage (e.g., sports drink, protein shake, protein shot, energy drink, caffeinated beverage, coffee drink (e.g., milk coffee), milk, fermented milk, smoothie, carbonated beverage, alcoholic beverage, infant formula, or meal replacement). The fat can be present in the food product in an amount of about 0.01% to about 5% by weight of the beverage. The beverage can contain no fat. The low flavor protein composition can have a luminance of at least 86 on a scale from 0 (black control value) to 100 (white control value). The low flavor protein composition can have a luminance of at least 88 on a scale from 0 (black control value) to 100 (white control value). The low flavor protein composition can have a chroma value of less than 14. The low flavor protein composition can have a chroma value of less than 12. The low flavor protein composition can have a chroma value of less than 10. The low flavor protein composition can have a chroma value of less than 8. The low flavor protein composition can have a chroma value of less than 6. The low flavor protein composition can include less than about 1.2% by dry weight lipids (e.g., less than about 1.0% or less than about 0.5% by dry weight lipids). The lipids can include one or more of a fatty acid, a wax, a sterol, a monoglyceride, a diglyceride, a triglyceride, a sphingolipid, or a phospholipid. The plurality of plant proteins, fungal proteins, algal proteins, bacterial proteins, protozoan proteins, invertebrate proteins, or a combination thereof can be at least 90% by dry weight soy proteins. The food product can further include at least one of a preservative, an antioxidant, or a shelf life extender. The low flavor protein composition can be in the form of a solution, suspension, or emulsion. The low flavor protein composition can be in the form of a solid or a powder. The low flavor protein composition can have an average particle size of about 5 µm to about 40 µm in the largest dimension. The low flavor protein composition can have an average particle size of about 5 µm to about 40 µm in the largest dimension. The low flavor protein composition can have an average particle size of about 10 µm to about 30 µm in the largest dimension. The low flavor protein composition can have an average particle size of about 10 µm to about 20 µm in the largest dimension. The low flavor protein composition can be in the form of an extrudate. The extrudate can be substantially in the form of granules. The granules can have an average largest dimension of about 3 mm to about 5 mm. Less than about 20% (w/w) of the granules can have a largest dimension less than 1 mm. Less than about 5% (w/w) of the granules can have a largest dimension over 1 cm. The extrudate can have a bulk density of about 0.25 to about 0.4 g/cm³. The extrudate can have a moisture content of about 5% to about 10%. The extrudate can have a protein content of about 65% to about 100% by dry weight. The extrudate can have a fat content of less than about 1.0%. The extrudate can have a sugar content of less than about 1%. The extrudate can have a hydration ratio of about 2.5 to about 3 after about 60 minutes of hydration at room temperature. The extrudate can have a hydration time of less than about 30 minutes. The extrudate can have a pH of about 5.0 to about 7.5 when hydrated. The extrudate can have a bite strength of about 2000 g to about 4000 g at a hydration ratio of about 3. In some embodiments, the low flavor protein composition has a protein dispersibility index of at least about 5 (e.g., at least about 10 or at least about 15). In some embodiments, the low flavor protein composition has a sodium level up to about 1 %w/w (e.g., up to about 0.5, up to about 0.1, up to about 0.05, up to about 0.01, or up to about 0.005 %w/w). The low flavor protein composition can have a solubility of at least 5% (e.g., at least 10%, at least 15%, at least 20%, at least 25%, or at least 30%) in an aqueous solution (e.g., water) or in the beverage. The aqueous solution or the beverage can have a pH of about 4.5 to about 8.0, of about 4.5 to about 7.0, of about 6.0 to about 8.0, of about 6.5 to about 7.5, of about 7.0 to about 8.0, of about 7.0, or of about 8.0. In some embodiments, the aqueous solution can include a buffer. In some embodiments, the low flavor protein composition exhibits a temperature-dependent change in one or more mechanical properties (e.g., storage modulus, loss modulus, and/or viscosity) over a temperature range (e.g., heating from 25° C. to 95° C., heating from 40° C. to 95° C., heating from 60° C. to 95° C., or heating from 80° C. to 90° C.). In some embodiments, the temperature-dependent change is at least 5-fold (e.g., at least 10-fold, at least 100-fold, at least 500-fold, or at least 1,000-fold) in magnitude. In some embodiments, the temperature-dependent change is substantially irreversible (e.g., upon cooling over the same temperature range, the magnitude of the change is up to 25%, up to 20%, up to 15%, up to 10%, up to 5%, up to 1%, up to 0.5%, or up to 0.1% the magnitude of the change observed upon heating). In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 90° C. In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 95° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 90° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 95° C. The low flavor protein composition can be a protein concentrate. The low flavor protein composition can be a protein isolate.

In yet another aspect, methods for preparing a food product are provided. Such methods generally include combining a fat, one or more optional flavor precursor compounds, and a low flavor protein composition, the low flavor protein composition produced by a method comprising: (a) adding an aqueous solution to a source protein composition to form a solution of solubilized protein; (b) optionally removing solids from the solution of solubilized protein; (c) adding an organic solvent to the solution of solubilized protein to form a solid phase and a liquid phase, and (d) separating the solid phase from the liquid phase to form a low flavor protein composition.

In still another aspect, methods for reducing perceived protein source flavor in a plant-based food product are provided. Such methods generally include combining a fat, one or more flavor precursor compounds and a low flavor protein composition, the low flavor protein composition produced by a method comprising: (a) adding an aqueous solution to a source protein composition to form a solution of solubilized protein; (b) optionally removing solids from the solution of solubilized protein; (c) adding an organic solvent to the solution of solubilized protein to form a solid phase and a liquid phase, and (d) separating the solid phase from the liquid phase to form a low flavor protein composition, wherein at least 5% by weight of the protein content of the food product can include the low flavor protein composition, thereby reducing perceived protein source flavor in a food product, as compared to a food product having a similar protein content but lacking the low flavor protein composition.

Implementations can include one or more of the following features. Step (a) can be performed at a pH of about 6.0 to about 9.0. Step (a) can be performed at a pH of about 7.5 to about 8.5. Step (a) can be performed at a pH of about 7.0 to about 11.0 (e.g., about 7.0 to about 10.0, about 8.0 to about 10.0, about 8.0 to about 9.0, or about 8.0). Step (b) can include centrifugation, filtration, or a combination thereof. Prior to step (c), the pH of the solution of solubilized protein can be adjusted to about 4.0 to about 9.0. Prior to step (c), the pH of the solution of solubilized protein can be adjusted to about 5.5 to about 7.5. Prior to step (c), the pH of the solution of solubilized protein can be adjusted to about 6.0 to about 7.0. Prior to step (c), the pH of the solution of solubilized protein can be adjusted to about 4.0 to about 7.0 (e.g., to about 4.0 to about 6.0, to about 4.5 to about 6.0, to about 4.5, or to about 6.0). In some embodiments, prior to step (c), the solution of solubilized protein is heated, for example, for about 10 seconds to about 30 minutes (e.g., about 10 seconds to about 20 minutes, about 10 seconds to about 30 seconds, about 10 seconds to about 1 minute, about 10 seconds to about 2 minutes, about 10 seconds to about 5 minutes, about 10 seconds to about 10 minutes, about 10 seconds to about 15 minutes, about 30 seconds to about 20 minutes, about 1 minute to about 30 minutes, about 1 minute to about 20 minutes, about 2 minutes to about 20 minutes, about 5 minutes to about 20 minutes, about 10 minutes to about 20 minutes, or about 15 minutes to about 20 minutes) at a temperature of about 70° C. to about 100° C. (e.g., about 80° C. to about 100° C., about 85° C. to about 100° C., about 85° C. to about 95° C., about 90° C. to about 100° C., about 85° C. to about 90° C., about 90° C. to about 95° C., or about 95° C. to about 100° C.). In some embodiments, prior to step (c), the organic solvent and/or the solution of solubilized protein are chilled, for example, to a temperature of about -20° C. to about 10° C. (e.g., about -20° C. to about 4° C.). In some embodiments, prior to step (c), the solution of solubilized protein is heated and then chilled. Step (c) can comprise adding an organic solvent. Step (c) can include adding the organic solvent to a final concentration of about 5% to about 70% (v/v). Step (c) can include adding the organic solvent to a final concentration of about 10% to about 50% (v/v). Step (c) can include adding the organic solvent to a final concentration of about 20% to about 30% (v/v). Step (c) can include adding the organic solvent to a final concentration of about 40% to about 90% (v/v) (e.g., to a final concentration of about 40% to about 70% (v/v), to a final concentration of about 40% to about 60% (v/v), or to a final concentration of about 45% to about 55% (v/v)). The pH can be adjusted by adding an acid. In some embodiments, the acid is selected from the group consisting of hydrochloric acid, acetic acid, citric acid, tartaric acid, malic acid, folic acid, fumaric acid, and lactic acid. In some embodiments, the acid is hydrochloric acid. Step (d) can include centrifugation, filtration, or a combination thereof. The organic solvent can be ethanol (e.g., at least 80%, at least 85%, at least 90%, at least 95%, at least 98%, at least 99%, at least 99.5%, or 100% ethanol). In some embodiments, the organic solvent is selected from the group consisting of ethanol, propanol, isopropyl alcohol, methanol, and acetone. The method further can include (e) washing the low flavor protein composition with an organic wash solvent. The method further can include (e) washing the low flavor protein composition with an aqueous wash solvent. The method further can include (e) washing the low flavor protein composition with first an organic wash solvent and second an aqueous wash solvent, or vice versa. The organic wash solvent can be ethanol (e.g., at least 80%, at least 85%, at least 90%, at least 95%, at least 98%, at least 99%, at least 99.5%, or 100% ethanol, or up to 20%, up to 15%, up to 10%, or up to 5% ethanol). In some embodiments, the organic wash solvent is selected from the group consisting of ethanol, propanol, isopropyl alcohol, methanol, and acetone. The organic wash solvent in step (e) can be the same as the organic solvent in step (c). The aqueous wash solvent can be water. In some embodiments, the aqueous wash solvent has a pH of about 6.0 to about 8.0, of about 6.5 to about 7.5, or of about 7.0. In some embodiments, the aqueous wash solvent can include a buffer. The method can further include drying the low flavor protein composition. Drying can include spray drying, mat drying, freeze-drying, or oven drying. The source protein composition can be at least 90% plant, algae, fungi, bacteria, protozoans, invertebrates, a part or derivative of any thereof, or a combination thereof on a dry weight basis. The food product can be a plant-based food product. The food product can be an algae-based food product. The food product can be a fungus-based food product. The food product can be an invertebrate-based food product. The fat can include at least one fat selected from the group consisting of corn oil, olive oil, soy oil, peanut oil, walnut oil, almond oil, sesame oil, cottonseed oil, rapeseed oil, canola oil, safflower oil, sunflower oil, flax seed oil, palm oil, palm kernel oil, coconut oil, babassu oil, shea butter, mango butter, cocoa butter, wheat germ oil, rice bran oil, and combinations thereof. The one or more flavor precursors comprise at least one compound selected from the group consisting of glucose, ribose, cysteine, a cysteine derivative, thiamine, alanine, methionine, lysine, a lysine derivative, glutamic acid, a glutamic acid derivative, IMP, GMP, lactic acid, maltodextrin, creatine, alanine, arginine, asparagine, aspartate, glutamic acid, glutamine, glycine, histidine, isoleucine, leucine, methionine, phenylalanine, proline, threonine, tryptophan, tyrosine, valine, linoleic acid, and mixtures thereof.

In any of the embodiments herein, the preservative, antioxidant, or shelf life extender can include at least one of 4-hexylresorcinol, acetic acid, ascorbic acid, ascorbyl palmitate, ascorbyl stearate, benzoic acid, butylated hydroxyanisole (a mixture of 2-tertiarybutyl-4-hydroxyanisole and 3-tertiarybutyl-4-hydroxyanisole), butylated hydroxytoluene (3,5-ditertiarybutyl-4-hydroxytoluene), calcium ascorbate, calcium propionate, calcium sorbate, Carnobacterium divergens M35, Carnobacterium maltaromaticum cb1, carnosum 4010, citric acid, a citric acid ester of a monoglyceride or diglyceride, dimethyl dicarbonate, erythorbic acid, ethyl lauroyl arginate, gum guaiacum, iso-ascorbic acid, L-cysteine, L-cysteine hydrochloride, lecithin, lecithin citrate, Leuconostoc, methyl paraben, methyl-p-hydroxybenzoate, monoglyceride citrate, monoisopropyl citrate, natamycin, nisin, potassium acetate, potassium benzoate, potassium bisulfite, potassium diacetate, potassium lactate, potassium metabisulfite, potassium nitrate, potassium nitrite, potassium sorbate, propionic acid, propyl gallate, propyl paraben, propyl-p-hydroxy benzoate, sodium acetate, sodium ascorbate, sodium benzoate, sodium bisulfite, sodium diacetate, sodium dithionite, sodium erythorbate, sodium iso-ascorbate, sodium lactate, sodium metabisulfite, sodium nitrate, sodium nitrite, sodium propionate, sodium salt of methyl-p-hydroxy benzoic acid, sodium salt of propyl-p-hydroxy benzoic acid, sodium sorbate, sodium sulfite, sorbic acid, sulfurous acid, tartaric acid, tertiary butyl hydroquinone, or a tocopherol.

As used herein, “low flavor” with respect to a protein composition means that the protein composition has less flavor than the source of the protein composition (e.g., soy, if a soy protein composition is described). For example, less (e.g., no more than 90%, 80%, 70%, 60%, 50%, 40%, 30%, 20%, or 10%) of one or more compounds that give rise to a distinguishing flavor associated with the source of the protein. In some embodiments, a low flavor protein composition can have little flavor of its own. In some instances, a low flavor protein composition has less flavor than a known protein composition (e.g., a commercial soy protein isolate, such as those described herein). Having less flavor can be determined, for example, by a trained human panelist, or, for example, by measurement of one or more volatile compounds commonly understood to impart flavor and/or aroma. In some embodiments, a low flavor protein composition can have a discriminability index of at least 1.0 (e.g., at least 1.5, 2.0, 2.5, or 3.0). In some embodiments, when assessed by a trained descriptive panel using the Spectrum method, a low flavor protein composition is described as having low intensity of one or more of: oxidized/rancid flavor, cardboard flavor, astringent flavor, bitter flavor, vegetable complex flavor, and sweet fermented flavor. In some embodiments, when assessed by a trained descriptive panel using the Spectrum method, a low flavor protein composition is described as having low intensity of one or more of: beany flavor, fatty flavor, green flavor, pea flavor, earthy flavor, hay-like flavor, grassy flavor, rancid flavor, leafy flavor, cardboard flavor, acrid flavor, pungent flavor, medicinal flavor, metallic flavor, and brothy flavor.

As used herein, “low color” with respect to a protein composition means that the protein composition has less color than the source of the protein composition (e.g., soy, if a soy protein composition is described). For example, less of one or more compounds that give rise to a color in the protein. In some embodiments, a low color protein composition can have little color of its own. In some instances, a low color protein composition has less color than a known protein composition (e.g., a commercial soy protein isolate, such as those described herein). Having less color can be determined, for example, by measuring the luminance and/or chroma of a protein composition. In some embodiments, a low color protein composition can have a luminance of at least about 86 (e.g., at least about 88, 90, 92, or 94). In some embodiments, a low color protein composition can have a chroma value of less than about 12 (e.g., less than about 10, 8, or 6).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the disclosure are set forth in the accompanying drawings and the description below. Other features, objects, and advantages of the disclosure will be apparent from the description and drawings, and from the claims. The word “comprising” in the claims may be replaced by “consisting essentially of” or with “consisting of,” according to standard practice in patent law.

### DESCRIPTION OF THE DRAWINGS

FIG. 1A is an exemplary flow chart for the preparation of a protein composition, according to some embodiments.

FIG. 1B is an exemplary flow chart for the preparation of a protein composition, according to some embodiments.

FIG. 1C shows exemplary phospholipid content of a protein composition prepared according some embodiments.

FIG. 1D shows exemplary protein content in supernatants according some embodiments.

FIG. 1E is an exemplary flow chart for the preparation of protein, according to some embodiments.

FIG. 2A shows exemplary data for the production of several soy flavor compounds, when an exemplary SPI produced as described herein is cooked in a flavor broth (referred to as FLB _EtOH), as compared to commercial products cSPC-1 and cSPI-1 and a control of the flavor broth alone (FLB).

FIG. 2B shows exemplary data for the production of several meat flavor compounds, when an exemplary SPI produced as described herein is cooked in a flavor broth (FLB _EtOH), as compared to commercial products cSPC-1 F and cSPI-1 and a control of the flavor broth alone (FLB).

FIG. 2C shows exemplary data for the production of several soy flavor compounds, when an exemplary SPI produced as described herein (pureSPI) and an exemplary SPC produced as described herein (pureSPC) are each cooked in water, as compared to commercial products cSPI-1, cSPI-2, cSPC-1, and cSPC-2.

FIG. 2D shows exemplary data for the production of several soy flavor compounds, when an exemplary SPI produced as described herein and an exemplary SPC produced as described herein are each cooked in a flavor broth (FLB_pureSPI and FLB_pureSPC, respectively), as compared to commercial products cSPI-1, cSPI-2, cSPC-1, and cSPC-2 and a control of the flavor broth alone (FLB).

FIG. 3A shows exemplary genistein content of some exemplary protein compositions produced as described herein.

FIG. 3B shows exemplary daidzein content of some exemplary protein compositions produced as described herein.

FIG. 3C shows exemplary glycitein content of some exemplary protein compositions produced as described herein.

FIG. 4A shows a comparison of two commercial SPCs (cSPC-1 and cSPC-2), two commercial SPIs (cSPI-1 and cSPI-2), and an exemplary SPC (pureSPC), produced as described herein, and an exemplary SPI (pureSPI), produced as described herein, on a black background.

FIG. 4B shows a comparison of two commercial SPCs (cSPC-1 and cSPC-2), two commercial SPIs (cSPI-1 and cSPI-2), and an exemplary SPC (pureSPC), produced as described herein, and an exemplary SPI (pureSPI), produced as described herein, on a white background.

FIG. 4C shows a comparison of commercial rapeseed protein isolate (cRPI) and an exemplary RPI (pureRPI), produced as described herein, on both a white and a black background.

FIG. 4D shows a comparison of starch, several commercial protein products, and an exemplary SPI (pureSPI), produced as described herein.

FIG. 4E shows a comparison of starting material (top row) versus exemplary protein compositions (bottom row) produced as described herein, including from soy, pea, canola, and spinach.

FIG. 4F shows a comparison of starting material (top row) versus exemplary protein compositions produced as described herein (bottom row), including from cricket, mealworm, beef, and yeast.

FIG. 4G shows a comparison of the color of an exemplary protein composition produced as described herein that has undergone different drying regimes.

FIG. 4H shows a comparison of the color of exemplary protein compositions produced under various conditions as described herein.

FIG. 5A is a bar plot of luminance data for various commercial protein products and exemplary corresponding protein compositions produced as described herein.

FIG. 5B is a bar plot of chroma data for various commercial protein products and exemplary corresponding protein compositions produced as described herein.

FIG. 6A shows the conditions of a hexad test for the evaluation of an exemplary protein composition produced as described herein.

FIG. 6B is a bar plot showing the results of the hexad test in FIG. 6A.

FIG. 7 shows exemplary milk replica beverages produced using a commercial soy protein isolate (cSPI-2) and an exemplary protein isolate (pureSPI) produced as described herein.

FIG. 8A shows microscopy images of an exemplary protein composition precipitated by ethanol (left) and an exemplary protein composition precipitated by acid (right).

FIG. 8B shows exemplary particle size distribution data for an exemplary protein composition precipitated by ethanol (single peak) and an exemplary protein composition precipitated by acid (double peak).

FIG. 9A shows the change of storage modulus and loss modulus of the cold -precipitated pureSPI with a temperature cycle between 25° C. and 95° C.

FIG. 9B shows the change of storage modulus and loss modulus of the room temperature-precipitated pureSPI with a temperature cycle between 25° C. and 95° C.

FIG. 9C shows the storage modulus of room temperature-precipitated pureSPI, cold -precipitated pureSPI, and commercial cSPI-3 at temperatures ranging from 25° C. and 95° C.

FIG. 10 shows a bar plot of the sodium levels in two commercial SPIs (cSPI-1 and cSPI-3) and an exemplary SPI (pureSPI), produced as described herein.

FIG. 11 shows a bar plot of the levels of isoflavone content, soyasaponin content, and phosphatidylcholine-36:4 content in two commercial SPIs (cSPI-2 and cSPI-3), three replicates of pureSPI, and soy flour. The y-axis is in ppm.

### DETAILED DESCRIPTION

This document is related to materials and methods for protein production. In particular, this document is related to materials and methods for the production of protein using precipitation. In general, this document provides protein compositions as well as methods and materials for purifying proteins resulting in protein compositions that can be used, for example, in food products, e.g., meat and dairy replica products or substitutes.

When a percentage is given herein, it means percent by dry weight, unless otherwise specified.

As used herein, the term “about” has its usual meaning in the context of the field of endeavor to allow for reasonable variations in amounts that can achieve the same effect and also refers herein to a value of plus or minus 10% of the provided value. For example, “about 20” means or includes amounts from 18 to and including 22.

A protein composition (e.g., a low flavor protein isolate, or a low color protein composition) as described herein can be produced from any suitable protein source composition. Non-limiting examples of protein source compositions include plants, algae, fungi, bacteria, protozoans, invertebrates, and a part or derivative of any thereof. As used herein a “part” of plants, algae, fungi, bacteria, protozoans, and invertebrates includes pieces of these, such as the leaves or stalks of plants, or the legs of invertebrates. As used herein, a “derivative” of plants, algae, fungi, bacteria, protozoans, and invertebrates includes products produced from these, such as freeze-dried plant leaves, commercial soy protein flours, concentrates, or isolates, or invertebrate meal.

Non-limiting examples of suitable plants include cottonwood (e.g., Celtis conferta), cottonseed (the seed of a cotton plant e.g., Gossypium hirsutum, Gossypium barbadense, Gossypium arboretum, Gossypium herbaceum, etc.), soybean (e.g., Glycine max), carob (e.g., Fabaceae sp.), peanut (e.g., Arachis hypogaea), mesquite (e.g., Prosopis sp.), lupin (e.g., Lupinus sp.), lentil (e.g., Lens culinaris, Lens esculenta, etc.), tamarind (e.g., Tamarindus indica), chickpea (e.g., Cicer arietinum), farrow (e.g., Triticum turgidum dicoccum), spelt (e.g., Triticum aestivum spelta), pea (e.g., Pisum sativum), alfalfa (e.g., Medicago sativa), clover (e.g., Trifolium sp.), bean (e.g., from the family Fabaceae), hemp (e.g., Cannabis sativa), hempseed (seeds of a hemp plant), sea beans (e.g., Salicornia sp.), rye (e.g., Secale cereal), sorghum (e.g., Sorghum spp.), teff (e.g., Eragrostis tef), freekeh (e.g., Triticum turgidum var. durum), quinoa (e.g., Chenopodium quinoa), rice (e.g., Oryza sativa), buckwheat (e.g., Fagopyrum esculentum), amaranth (e.g., Amaranthus cruentus), barley (e.g., Hordeum vulgare), corn (e.g., Zea mays), bulgur wheat (e.g., Triticum ssp.), einkorn wheat (e.g., Triticum monococcum), wheat (e.g., Triticum aestivum, Triticum turgidum, etc.), wild rice (e.g., Zizania spp.), khorasan grain (e.g., triticum turgidum turanicum), millet (e.g., Panicum miliaceum, Pennisetum Glaucum, Setaria italica, eleusine coracana, digitaria exilis, etc.), chia seed (e.g., Salvia hispanica), oat (e.g., Avena sativa), triticale (e.g., × Triticosecale), lucerne, cassava (e.g., Manihot esculenta), lablab bean (e.g., Lablab purpureus), moringa oleifera, collards (e.g., Brassica oleracea), stinging nettle (e.g., Urtica dioica), moss (from the division Bryophyta sensu stricto), bamboo (e.g., from the family Bambusoideae), among others. Plants can include legumes and pulses.

Non-limiting examples of suitable algae include cyanobacteria (e.g., blue-green algae) such as spirulina (e.g., Arthrospira platensis, Arthrospira maximus, etc.), species from the genus chlorella, and Aphanizomenonflos-aquae. Some algae is multicellular and include seaweeds such as Rhodophyta (red algae), Chlorophyta or Charophyta/Streptophyta (green algae), Phaeophyceae (brown algae). Some examples of red algae can include species from the genus Porphyra (nori), and Palmaria palmate (dulse). Some examples of green algae can include Caulerpa lentillifera (seagrapes), Ulva lactuca (sea lettuce), and Chlamydomonas reinhardtii. Some examples of brown algae include Macrocystis (kelp), Sargassum (seaweed mats), brown algae from the order Fucales, and Ascophyllum nodosum, (e.g., macrocystis).

Non-limiting examples of suitable fungi include brewer’s yeast (e.g., nutritional yeast, Saccharomyces cerevisiae, etc.), Brettanomyces bruxellensis, Brettanomyces anomalus, Brettanomyces custersianus, Brettanomyces naardenensis, Brettanomyces nanus, Dekkera bruxellensis, Dekkera anomala, Candida stellata, Schizosaccharomyces pombe, Torulaspora delbrueckii, Zygosaccharomyces bailii, Pichia pastoris (also called, in some cases, Komagataella phaffii, K. pastoris, or K. pseudopastoris). Some suitable fungi may include mycoprotein derived from Fusarium venenatum. Other types of suitable fungi may include edible mushroom varieties such as Agaricus bisporus, Pleurotus ostreatus, Lentinula edodes, Auricularia auricula-judae, Volvariella volvacea, Flammulina velutipes, Tremella fuciformis, Hypsizygus tessellatus, Stropharia rugosoannulata, Cyclocybe aegerita, Hericium erinaceus, Boletus edulis, Calbovista subsculpta, Calvatia gigantean, Cantharellus cibarius, Craterellus tubaeformis, Clitocybe nuda, Cortinarius caperatus, Craterellus cornucopioides, Grifola frondosa, Gyromitra esculenta, Hericium erinaceus, Hydnum repandum, Lactarius deliciosus, Morchella, Pleurotus ostreatus, Tricholoma matsutake, Tuber sp. among others.

Non-limiting examples of suitable bacteria include methanotrophs (e.g., Methylococcus capsulatus), Methylophilus methylotrphus, Rhodobacter capsulatus bacterial species that are capable of producing syngas fermentation (e.g., homoacetogenic clostridia sp.), among others. some examples of suitable bacteria can be bacterial species that are capable of producing single -cell protein such as Bacillus cereus, Bacillus licheniformis, Bacillus pumilis, Bacillus subtilis, Corynobacterium ammoniagenes, Corynebacterium glutamicum, Cupriavidus necator, Escherichia coli, Haloarcula sp. IRU1, Ralstonia sp., Brevibacillus agri, Aneurunibacillus sp., Methylomonas sp., Rhizosperic diazotrophs, Rhodopseudomonas palustris, among others.

Non-limiting examples of suitable protozoans include Trichonympha, Pyrsonympha, Trichomonas, Isotricha, Entodinium, among others.

Non-limiting examples of suitable invertebrates include spider species (e.g., Haplopelma albostriatum), other arthropods such as scorpions (e.g., Typhlochactas mitchelli, Heterometrus swammerdami, etc.), cricket (e.g., from the order Orthoptera), ants (e.g., from the order Hymenoptera), silkworm and/or moths (e.g., from the order Lepidoptera), beetles (e.g., from the order Coleoptera, flies (e.g., from the order Diptera), among others.

In one aspect, provided herein are methods of preparing a protein composition. In some embodiments, a protein composition can be a protein concentrate. In some embodiments, a protein composition can be a protein isolate. In some embodiments, a protein composition can be a low flavor protein isolate. In some embodiments, a protein composition can be a low color protein composition.. In some embodiments, a protein composition can be a low color protein composition that is a protein concentrate. In some embodiments, a protein composition can be a low color protein composition that is a protein isolate. In some embodiments, a protein composition can be a low flavor and low color protein composition that is a protein isolate.

In some cases, the methods described herein can include one or more steps or conditions that help preserve and/or increase the functionality of the protein in the protein composition. As described herein, functional proteins can have one or more (e.g., two or more, three or more, four or more, or five or more) of the following properties: has a protein dispersibility index of at least about 5 (e.g., at least about 10 or at least about 15); has a sodium level up to about 1 %w/w (e.g., up to about 0.5, up to about 0.1, up to about 0.05, up to about 0.01, or up to about 0.005 %w/w); has a solubility of at least 5% (e.g., at least 10%, at least 15%, at least 20%, at least 25%, or at least 30%) in an aqueous solution (e.g., water), where the aqueous solution can have a pH of about 6.0 to about 8.0, of about 6.5 to about 7.5, of about 7.0 to about 8.0, of about 7.0, or of about 8.0 and/or the aqueous solution can include a buffer; exhibits a temperature-dependent change in one or more mechanical properties (e.g., storage modulus, loss modulus, and/or viscosity) over a temperature range (e.g., heating from 25° C. to 95° C., heating from 40° C. to 95° C., heating from 60° C. to 95° C., or heating from 80° C. to 90° C.), where the temperature-dependent change can be at least 5-fold (e.g., at least 10-fold, at least 100-fold, at least 500-fold, or at least 1,000-fold) in magnitude, the temperature-dependent change can be substantially irreversible (e.g., upon cooling over the same temperature range, the magnitude of the change can be up to 25%, up to 20%, up to 15%, up to 10%, up to 5%, up to 1%, up to 0.5%, or up to 0.1% the magnitude of the change observed upon heating), the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 90° C., the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 95° C., the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 90° C., and/or the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 95° C.; capable of forming a gel upon heating (e.g., a suspension of about 25 to about 250 mg/mL (e.g., about 25 to about 50 mg/mL, about 25 to about 100 mg/mL, about 25 to about 150 mg/mL, about 25 to about 200 mg/mL, about 50 to about 250 mg/mL, about 100 to about 250 mg/mL, about 150 to about 250 mg/mL, or about 200 to about 250 mg/mL) at a pH of about 7.0); thermally transitions to a gel upon heating to about 65° C.; thermally denatures during incubation between about 50° C. and about 85° C., with greater than about 80% of the protein denaturing after about 20 minutes at about 85° C., as measured either by differential scanning calorimetry (DSC) or differential scanning fluorimetry (DSF); in a solution or suspension of purified protein at or above about 50 mg/mL (5% w/v), protein forms a freestanding gel (with, e.g., a 100 Pa storage modulus) when heated at or above about 85° C. for about 20 minutes; can denature and gel between about pH 5.5 and about pH 10.0; can denature and gel in solutions with ionic strength (I) below about 0.5 M, when I is calculated based on the concentration of non-protein solutes; at a protein concentration of about 10 mg/mL, particle size distribution D10, D50 and D90 are less than about 0.1 µm, 1.0 µm and 5 µm, respectively; has enzymatic activity; or has an emulsion activity index (EAI) of greater than or equal to about 50 m²/gprotein across a pH range of about 4.0 to about 8.0.

In some embodiments, the method for making a protein composition comprises: (a) adding an aqueous solution to a source protein composition to form a solution of solubilized protein; (b) optionally removing solids from the solution of solubilized protein; (c) adding an organic solvent to the solution of solubilized protein to form a solid phase and a liquid phase, and (d) separating the solid phase from the liquid phase to form a protein composition including a plurality of plant proteins, fungal proteins, algal proteins, bacterial proteins, protozoan proteins, invertebrate (e.g., insect and/or arachnid) proteins, or a combination thereof

In some embodiments of any of the methods described herein, an aqueous solution can be added to a source protein composition to form a solubilized protein. In some embodiments, a protein composition can be in the form of a solid (e.g., a powder), a suspension, a solution, or an emulsion). An aqueous solution, can in some embodiments, be water. In some embodiments, an aqueous solution can include a buffer. The buffer can be any food-grade buffer (e.g., a buffer that includes sodium phosphate, potassium phosphate, calcium phosphate, sodium acetate, potassium acetate, sodium citrate, calcium citrate, sodium bicarbonate, sodium lactate, potassium lactate, sodium malate, potassium malate, sodium gluconate, and/or potassium gluconate) at a concentration of about 2 mM to about 200 mM (e.g., about 2 mM to about 10 mM, about 10 mM to about 20 mM, about 10 mM to about 30 mM, about 20 mM to about 30 mM, about 30 mM to about 40 mM, about 40 mM to about 50 mM, about 50 mM to about 100 mM, or about 100 mM to about 200 mM). An aqueous solution can include any other appropriate components (e.g., a salt, such as sodium chloride or potassium chloride).

A source protein composition can be any suitable source protein composition. In some embodiments, a source protein composition can be at least 90% plants, algae, fungi, bacteria, protozoans, invertebrates, a part or derivative of any thereof, or a combination thereof on a dry weight basis. In some embodiments, a source protein composition can be at least 90% plants, a part or derivative of any thereof, or a combination thereof on a dry weight basis. In some embodiments, a source protein composition can be at least 90% algae, a part or derivative of any thereof, or a combination thereof on a dry weight basis. In some embodiments, a source protein composition can be at least 90% fungi, a part or derivative of any thereof, or a combination thereof on a dry weight basis. In some embodiments, a source protein composition can be at least 90% bacteria, a part or derivative of any thereof, or a combination thereof on a dry weight basis. In some embodiments, a source protein composition can be at least 90% protozoans, a part or derivative of any thereof, or a combination thereof on a dry weight basis. In some embodiments, a source protein composition can be at least 90% invertebrates, a part or derivative of any thereof, or a combination thereof on a dry weight basis. In some embodiments, a source protein composition can be defatted. In some embodiments, a source protein composition can be a flour or a flake (e.g., soy white flakes). In some embodiments, the source protein composition can be at least 90% a defatted soy flour, a defatted pea flour, or a combination thereof on a dry weight basis.

In some embodiments, the pH of the solution of solubilized protein can have a pH of about 4.0 to about 9.0 (e.g., about 4.0 to about 8.0, about 4.0 to about 7.0, about 4.0 to about 6.0, about 4.0 to about 5.0, about 5.0 to about 9.0, about 6.0 to about 9.0, about 7.0 to about 9.0, about 8.0 to about 9.0). In some embodiments, an aqueous solution can have a pH of about 7.5, about 8.0, or about 8.5. In some embodiments, the pH of the solution of solubilized protein can have a pH of about 6.0 to about 9.0. In some embodiments, the pH of the solution of solubilized protein can have a pH of about 7.5 to about 8.5. In some embodiments, the pH of the solution of solubilized protein can have a pH of about 7.0 to about 11.0 (e.g., about 7.0 to about 10.0, about 8.0 to about 10.0, about 8.0 to about 9.0, or about 8.0).

In some cases, the pH can fall in this range without adjustment. For example, the pH can fall into the mentioned range responsive to the addition of an aqueous solution to the source protein to create a solution of solubilized protein. In some cases, the pH can be adjusted to fall in this range. In some embodiments, an acid (e.g., hydrochloric acid, acetic acid, citric acid, tartaric acid, malic acid, folic acid, fumaric acid, lactic acid, etc.) can be added to the solution of solubilized protein to decrease the pH. In other embodiments, a base (e.g., potassium hydroxide, sodium hydroxide, etc.) can be added to the solution of solubilized protein to increase the pH. In other embodiments, the pH can fall into the mentioned range responsive to a combination of acid(s) and base(s) added to the solution of solubilized protein. In yet other embodiments, the pH can remain in the mentioned pH range responsive to a buffer (e.g., [tris(hydroxymethyl)methylamino]propanesulfonic acid, 2-(bis(2-hydroxyethyl)amino)acetic acid, etc.) added to the solution of solubilized protein.

In some embodiments, the pH of the solution of solubilized protein can be adjusted by the addition of an acid and/or a base. In some embodiments, the pH of the solution of solubilized protein can be adjusted to about 4.0 to about 9.0 (e.g., about 4.0 to about 8.0, about 4.0 to about 7.0, about 4.0 to about 6.0, about 4.0 to about 5.0, about 5.0 to about 9.0, about 6.0 to about 9.0, about 7.0 to about 9.0, about 8.0 to about 9.0). In some embodiments, the pH of the solution of solubilized protein can be adjusted to about 4.0 to about 5.0. In other some embodiments, the pH of the solution of solubilized protein can be adjusted to about 4.5. In some embodiments, the pH of the solution of solubilized protein can be adjusted to about 5.5 to about 7.5. In other some embodiments, the pH of the solution of solubilized protein can be adjusted to about 5.5 to about 6.5. In some embodiments, the pH of the solution of solubilized protein is adjusted to about 6.0 to about 7.0. In some embodiments, the pH of the solution of solubilized protein can be adjusted to about 5.5, 6.0, 6.5, or 7.0.

Optionally, solids can be removed from the solution of solubilized protein. Solids can be removed by any suitable means. In some embodiments, solids can be removed with centrifugation, filtration, or a combination thereof. In some embodiments, the removal of solids can include refraining from agitation for a threshold period of time, and aspirating a liquid portion from the solution of solubilized protein. For example, the solution of solubilized protein can be positioned undisturbed for a threshold period of time such that any solids from the solution of solubilized protein can settle on the bottom of a container. In this instance, the liquid from the solution of solubilized protein can be aspirated to remove said liquid from the solid that is settled on the bottom of said container. In some examples, a combination of refraining from agitation for a threshold period of time can be combined with other methods such as centrifugation and/or filtration. Specifically, in some examples, the solution of solubilized protein can be left undisturbed for a threshold period of time, a liquid portion can be removed from the undisturbed solution of solubilized protein and filtered and/or centrifuged to further remove solids from the solution of solubilized protein.

In some embodiments, a solution of solubilized protein can be heated before an organic solvent and/or acid is added to the solution of solubilized protein. Without being bound by any particular theory, it is believed that heating the solution of solubilized protein can result in the formation of larger protein structures (e.g., larger flocs, or aggregates of particles with a cheese-curd-like structure) and/or the disruption of intermolecular interactions between proteins and other components (e.g., fats, carbohydrates, or small molecules such as flavor compounds or pigments). The solution of solubilized protein can be heated for any appropriate amount of time, for example, about 10 seconds to about 30 minutes (e.g., about 10 seconds to about 20 minutes, about 10 seconds to about 30 seconds, about 10 seconds to about 1 minute, about 10 seconds to about 2 minutes, about 10 seconds to about 5 minutes, about 10 seconds to about 10 minutes, about 10 seconds to about 15 minutes, about 30 seconds to about 20 minutes, about 1 minute to about 30 minutes, about 1 minute to about 20 minutes, about 2 minutes to about 20 minutes, about 5 minutes to about 20 minutes, about 10 minutes to about 20 minutes, or about 15 minutes to about 20 minutes). The solution of solubilized protein can be heated at any appropriate temperature, for example, about 70° C. to about 100° C. (e.g., about 80° C. to about 100° C., about 85° C. to about 100° C., about 85° C. to about 95° C., about 90° C. to about 100° C., about 85° C. to about 90° C., about 90° C. to about 95° C., or about 95° C. to about 100° C.).

In some embodiments, a solution of solubilized protein and/or an organic solvent can be chilled before an organic solvent and/or acid is added to the solution of solubilized protein. The solution of solubilized protein and/or an organic solvent can be chilled, for example, to a temperature of about -20° C. to about 10° C. (e.g., about -20° C. to about 4° C.). In some embodiments, a solution of solubilized protein is heated and then chilled before an organic solvent and/or acid is added to the solution of solubilized protein.

An organic solvent can be added to the solution of solubilized protein. The addition of an organic solvent can form (e.g., precipitate) a solid phase (e.g., a protein composition) from a liquid phase of the solution of solubilized protein. Non-limiting examples of suitable organic solvents can include methanol, propanol, isopropanol, EtOH (ethanol), and acetone. For example, an organic solvent can be added to a final concentration of about 5% to about 70% (v/v) (e.g., about 5% to about 10% (v/v), about 5% to about 20% (v/v), about 5% to about 30% (v/v), about 5% to about 40% (v/v), about 5% to about 50% (v/v), about 5% to about 60% (v/v), about 10% to about 70% (v/v), about 20% to about 70% (v/v), about 30% to about 70% (v/v), about 40% to about 70% (v/v), about 50% to about 70% (v/v), about 60% to about 70% (v/v), about 20% to about 50% (v/v), about 20% to about 30% (v/v), about 30% to about 40%, or about 50% to about 60% (v/v)). In some embodiments, methanol can be added to a final concentration of about 5% to about 70% (v/v) (e.g., about 5% to about 10% (v/v), about 5% to about 20% (v/v), about 5% to about 30% (v/v), about 5% to about 40% (v/v), about 5% to about 50% (v/v), about 5% to about 60% (v/v), about 10% to about 70% (v/v), about 20% to about 70% (v/v), about 30% to about 70% (v/v), about 40% to about 70% (v/v), about 50% to about 70% (v/v), about 60% to about 70% (v/v), about 20% to about 50% (v/v), about 20% to about 30% (v/v), about 30% to about 40%, or about 50% to about 60% (v/v)). In some embodiments, isopropanol can be added to a final concentration of about 5% to about 70% (v/v) (e.g., about 5% to about 10% (v/v), about 5% to about 20% (v/v), about 5% to about 30% (v/v), about 5% to about 40% (v/v), about 5% to about 50% (v/v), about 5% to about 60% (v/v), about 10% to about 70% (v/v), about 20% to about 70% (v/v), about 30% to about 70% (v/v), about 40% to about 70% (v/v), about 50% to about 70% (v/v), about 60% to about 70% (v/v), about 20% to about 50% (v/v), about 20% to about 30% (v/v), about 30% to about 40%, or about 50% to about 60% (v/v)). In some embodiments, EtOH can be added to a final concentration of about 5% to about 70% (v/v) (e.g., about 5% to about 10% (v/v), about 5% to about 20% (v/v), about 5% to about 30% (v/v), about 5% to about 40% (v/v), about 5% to about 50% (v/v), about 5% to about 60% (v/v), about 10% to about 70% (v/v), about 20% to about 70% (v/v), about 30% to about 70% (v/v), about 40% to about 70% (v/v), about 50% to about 70% (v/v), about 60% to about 70% (v/v), about 20% to about 50% (v/v), about 20% to about 30% (v/v), about 30% to about 40%, or about 50% to about 60% (v/v)). In some embodiments, acetone can be added to a final concentration of about 5% to about 70% (v/v) (e.g., about 5% to about 10% (v/v), about 5% to about 20% (v/v), about 5% to about 30% (v/v), about 5% to about 40% (v/v), about 5% to about 50% (v/v), about 5% to about 60% (v/v), about 10% to about 70% (v/v), about 20% to about 70% (v/v), about 30% to about 70% (v/v), about 40% to about 70% (v/v), about 50% to about 70% (v/v), about 60% to about 70% (v/v), about 20% to about 50% (v/v), about 20% to about 30% (v/v), about 30% to about 40%, or about 50% to about 60% (v/v)). In some embodiments, the pH of the solution of solubilized protein can be about 6.0, and the final concentration of the organic solvent (e.g., ethanol) can be about 5% to about 70% (v/v) (e.g., about 5% to about 10% (v/v), about 5% to about 20% (v/v), about 5% to about 30% (v/v), about 5% to about 40% (v/v), about 5% to about 50% (v/v), about 5% to about 60% (v/v), about 10% to about 70% (v/v), about 20% to about 70% (v/v), about 30% to about 70% (v/v), about 40% to about 70% (v/v), about 50% to about 70% (v/v), about 60% to about 70% (v/v), about 20% to about 50% (v/v), about 20% to about 30% (v/v), about 30% to about 40%, or about 50% to about 60% (v/v)). In some embodiments, the pH of the solution of solubilized protein can be about 6.0, and the final concentration of the organic solvent (e.g., ethanol) can be about 50%. In some embodiments, the pH of the solution of solubilized protein can be about 4.5 to about 6.0, and the final concentration of the organic solvent (e.g., ethanol) can be about 40% to about 70%. In some embodiments, the pH of the solution of solubilized protein can be about 6.0, and the final concentration of the organic solvent (e.g., ethanol) can be about 40% to about 70%. In some embodiments, the pH of the solution of solubilized protein can be about 4.5, and the final concentration of the organic solvent (e.g., ethanol) can be about 25% (v/v). In some embodiments, the organic solvent does not include carbon dioxide (e.g., supercritical carbon dioxide).

The organic solvent can be added to the solution of solubilized protein at any appropriate temperature. In some embodiments, the organic solvent can be added to the solution of solubilized protein at approximately ambient temperature (e.g., room temperature). In some embodiments, the organic solvent can be added to the solution of solubilized protein at a temperature of about 10° C. to about 25° C. (e.g., about 10° C. to about 15° C., about 10° C. to about 20° C., about 15° C. to about 25° C., or about 20° C. to about 25° C.). In some embodiments, the organic solvent can be chilled. Without being bound by any particular theory, it is believed that using a chilled organic solvent may help to preserve some of the functionality of the protein. In some embodiments, the organic solvent can be added to the solution of solubilized protein at a temperature of about -20° C. to about 10° C. (e.g., about -20° C. to about -10° C., about -20° C. to about 0° C., about -20° C. to about 4° C., about -10° C. to about 10° C., about 0° C. to about 10° C., or about 4° C. to about 10° C.).

An acid can be added to the solution of solubilized protein. The addition of an acid can form (e.g., precipitate) a solid phase (e.g., a protein composition) from a liquid phase of the solution of solubilized protein. In some embodiments, the acid is selected from the group consisting of hydrochloric acid, acetic acid, citric acid, tartaric acid, malic acid, folic acid, fumaric acid, and lactic acid. In some embodiments, the acid is hydrochloric acid.

The solution of solubilized protein can be at any appropriate temperature when the organic solvent and/or acid is added. In some embodiments, the solution of solubilized protein can be approximately ambient temperature (e.g., room temperature) when the organic solvent is added. In some embodiments, the solution of solubilized protein can be at temperature of about 10° C. to about 25° C. (e.g., about 10° C. to about 15° C., about 10° C. to about 20° C., about 15° C. to about 25° C., or about 20° C. to about 25° C.) when the organic solvent is added. In some embodiments, the solution of solubilized protein can be chilled when the organic solvent is added. Without being bound by any particular theory, it is believed that having the solution of solubilized protein chilled when the organic solvent is added may help to preserve some of the functionality of the protein. In some embodiments, the solution of solubilized protein can be at a temperature of about 2° C. to about 10° C. (e.g., about 2° C. to about 4° C., about 2° C. to about 5° C., about 2° C. to about 8° C., about 4° C. to about 10° C., about 5° C. to about 10° C., or about 8° C. to about 10° C.).

Separation of the precipitated protein (solid phase) from the solution (liquid phase) can be achieved by any suitable method to form a protein composition (e.g., a low flavor protein composition or a low color protein composition). In some embodiments, the solid phase can be removed with centrifugation, filtration, or a combination thereof. In other embodiments, the removal of the solid phase can include refraining from agitation for a threshold period of time, and aspirating the liquid phase from the away from the solid phase. For example, the solution of solubilized protein (including the organic solvent) can be positioned undisturbed for a threshold period of time such that the solid phase from the solution of solubilized protein can settle on the bottom of a container. In this instance, the liquid phase from the solution of solubilized protein can be aspirated to remove said liquid phase from the solid phase that is settled on the bottom of said container. In another example, a combination of refraining from agitation for a threshold period of time can be combined with other methods such as centrifugation and/or filtration. Specifically, in some examples, the solution of solubilized protein can be left undisturbed for a threshold period of time, the liquid phase can be removed from the undisturbed solution of solubilized protein and filtered and/or centrifuged to further remove any remaining solid phase portions from the aspirated liquid phase.

A protein composition (e.g., the solid phase) can optionally be washed with one or more wash solvents (e.g., an organic wash solvent, an aqueous wash solvent (e.g., water, or a buffer), or a mixture of an aqueous wash solvent (e.g., water) and an organic wash solvent). In some embodiments, a wash solvent can be a mixture of water and an organic wash solvent, for example, the wash solvent can include 0%, 5%, 10%, 20%, 30%, 40%, 50%, 60%, 70%, 80%, or 90% of the organic wash solvent (v/v). Non-limiting examples of suitable organic wash solvents can include methanol, propanol, isopropanol, EtOH, and acetone. An organic wash solvent can be used to wash the solid phase, containing precipitated protein. In some embodiments, an organic wash solvent can be the same organic solvent as was used for precipitation. In some embodiments, an organic wash solvent can be a different organic solvent as was used for precipitation. In some cases, the wash step can be repeated one or more times, with the wash solvent independently selected (e.g., from those described herein) for each wash step repetition. For example, in some embodiments, a wash solvent for a first wash step can include about 70% to about 100% (v/v) ethanol, and a repeated wash step can use a wash solvent that can include about 0% to about 20% (v/v) ethanol. A protein composition (e.g., the solid phase) can optionally be washed with first an organic wash solvent and second an aqueous wash solvent, or vice versa.

In some cases, a protein composition (e.g., before resolubilization) can have a protein dispersibility index of about 3 to about 20 (e.g., about 3 to about 18, about 3 to about 15, about 3 to about 12, about 3 to about 10, about 3 to about 8, about 3 to about 5, about 5 to about 20, about 8 to about 20, about 10 to about 20, about 12 to about 20, about 15 to about 20, about 18 to about 20, about 5 to about 15, or about 8 to about 12).

In some embodiments of any of the methods described herein, a protein composition can be treated (e.g., after being optionally washed). A non-limiting example of treatment is resolubilization.

In some cases, a protein composition can be at least partially resolubilized. Without being bound by any particular theory, it is believed that at least partial resolubilization can result in the protein composition having increased functionality or being easier to use in food applications. In some embodiments, resolubilized protein can be soluble at a concentration of about 1.5 to about 50 mg/mL (e.g., about 1.5 to about 5.0 mg/mL, about 1.5 to about 4.0 mg/mL, about 2.0 to about 4.0 mg/mL, about 1.5 to about 20 mg/mL, about 1.5 to about 10 mg/mL, about 10 to about 50 mg/mL, about 10 to about 40 mg/L, about 10 to about 30 mg/mL, about 10 to about 20 mg/mL, about 20 to about 50 mg/mL, or about 20 to about 40 mg/mL). In some embodiments, a pH change can be used to solubilize the protein composition. In some embodiments, the pH of the protein composition can be adjusted to at least 7 (e.g., at least 8, at least 9, at least 10, or at least 11). In some embodiments, the protein composition can be further neutralized (e.g., brought to a pH of about 6.0 to about 8.0, about 6.5 to about 7.5, or about 7.0) after the pH change. In some embodiments, an enzyme can be used to solubilize the protein, for example, a protein glutaminase, a protein asparaginase, or a protein deamidase.

A protein composition can be dried. The protein composition can be dried by any suitable method. For example, the protein composition can be dried via spray drying, mat drying, freeze-drying (e.g., lyophilizing), oven-drying (e.g., at about 70° C. to about 90° C., such as about 80° C.), and combinations thereof.

Accordingly, provided herein are methods of preparing a protein composition, the method including (a) adding an aqueous solution to a source protein composition to form a solution of solubilized protein; (b) optionally removing solids from the solution of solubilized protein; (c) optionally heating the solution of solubilized protein; (d) optionally adjusting the pH of the solution of solubilized protein to about 4.0 to about 9.0; (e) optionally cooling the solution of solubilized protein to about 0° C. to about 10° C.; (f) adding an organic solvent to the solution of solubilized protein to form a solid phase and a liquid phase; (g) separating the solid phase from the liquid phase to form the protein composition; (h) optionally washing the protein composition with a wash solvent; and (i) optionally treating the protein composition, wherein the protein composition comprises at least at least 50% by dry weight of a plurality of plant proteins, fungal proteins, algal proteins, bacterial proteins, protozoan proteins, invertebrate proteins.

In some embodiments, the methods can include steps (a), (b), (f), and (g). In some embodiments, the methods can include steps (a), (b), (c), (f), and (g). In some embodiments, step (c) follows step (b). In some embodiments, step (b) follows step (c). In some embodiments, the methods can include steps (a), (b), (d), (f), and (g). In some embodiments, step (d) follows step (b). In some embodiments, the methods can include steps (a), (b), (e), (f), and (g). In some embodiments, step (e) follows step (b). In some embodiments, step (b) follows step (e). In some embodiments, the methods can include steps (a), (b), (c), (d), (f), and (g). In some embodiments, steps (b), (c), and (d) are performed in the order of (b), (c), (d). In some embodiments, (b), (c), and (d) are performed in the order of (c), (b), (d). In some embodiments, steps (b), (c), and (d) are performed in the order of (b), (d), (c). In some embodiments, the methods can include steps (a), (b), (c), (e), (f), and (g). In some embodiments, steps (b), (c), and (e) are performed in the order of (b), (c), (e). In some embodiments, steps (b), (c), and (e) are performed in the order of (c), (b), (e). In some embodiments, steps (b), (c), and (e) are performed in the order of (b), (e), (c). In some embodiments, the methods can include steps (a), (b), (c), (d), (e), (f), and (g). In some embodiments, steps (b), (c), (d), and (e) are performed in the order of (b), (c), (d), (e). In some embodiments, steps (b), (c), (d), and (e) are performed in the order of (c), (b), (d), (e). In some embodiments, steps (b), (c), (d), and (e) are performed in the order of (b), (d), (e), (c). In some embodiments, steps (b), (c), (d), and (e) are performed in the order of (b), (d), (c), (e). In some embodiments, the methods can include steps (a), (c), (f), and (g). In some embodiments, the methods can include steps (a), (c), (d), (f), and (g). In some embodiments, step (c) is performed before step (d). In some embodiments, step (d) is performed before step (c). In some embodiments, the methods can include steps (a), (c), (d), (e), (f), and (g). In some embodiments, steps (c), (d), and (e) are performed in the order (c), (d), (e). In some embodiments, steps (c), (d), and (e) are performed in the order (d), (e), (c). In some embodiments, steps (c), (d), and (e) are performed in the order (d), (c), (e). In some embodiments, the methods can include steps (a), (d), (f), and (g). In some embodiments, the methods can include steps (a), (d), (e), (f), and (g). In some embodiments, step (d) is performed before step (e). In some embodiments, the methods can include steps (a), (e), (f), and (g). In some embodiments of any of the methods described herein, the methods can include step (h). In some embodiments, step (h) is repeated one or more times. In some embodiments, in a repeat of step (h), the wash solvent is the same as in the first step (h). In some embodiments, in a repeat of step (h), the wash solvent is different than in the first step (h). In some embodiments of any of the methods described herein, the methods can include step (i). In some embodiment, the methods can further include drying the protein composition. In some embodiments, the drying can include spray drying, mat drying, freeze-drying, or oven drying.

In some embodiments, the source protein composition can include one or more isoflavones. In some embodiments, the source protein composition can be a soy source protein composition and can include one or more isoflavones (e.g., genistein, daidzein, glycitein, or a combination thereof). In some embodiments, the methods described herein can result in the reduction in content of one or more isoflavones in the protein composition as compared to the source protein composition. For instance, the protein composition can have an isoflavone content less than 90% (e.g., less than 80%, 70%, 60%, 50%, 40%, 30%, 20%, 10%, or less) of the isoflavone content of the source protein composition, on a dry weight basis.

In some embodiments, the source protein composition can include one or more sphingolipids, disaccharides (e.g., sucrose), oligosaccharides (e.g., raffinose, stachyose), phytoestrogens, lignans, O-methylated isoflavones (e.g., formononetin, biochanin A), phytoalexins, coumestans (e.g., coumestrol), phytotoxins, phytochemicals, carotenoids, or pterocarpans (e.g., glycinol, glyceollidin I and II, glyceollins (glyceollin I, II, III and IV)). In some embodiments, the methods described herein can result in the reduction in content of one or more sphingolipids, disaccharides (e.g., sucrose), oligosaccharides (e.g., raffinose, stachyose), phytoestrogens, lignans, O-methylated isoflavones (e.g., formononetin, biochanin A), phytoalexins, coumestans (e.g., coumestrol), phytotoxins, phytochemicals, carotenoids, or pterocarpans (e.g., glycinol, glyceollidin I and II, glyceollins (glyceollin I, II, III and IV)) in the protein composition as compared to the source protein composition. For instance, the protein composition can have a content less than 90% (e.g., less than 80%, 70%, 60%, 50%, 40%, 30%, 20%, 10%, or less) of the content of the source protein composition, on a dry weight basis.

A protein composition can, in some embodiments, produce less of one or more flavor compounds (e.g., soy flavor compounds) when cooked as compared to the amount of the one or more flavor compounds (e.g., soy flavor compounds) produced by cooking the source protein composition. Non-limiting examples of the one or more of the flavor compounds (e.g., soy flavor compounds) are hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal. For example, when cooked in water, a 1% (w/v) suspension of a protein composition (by dry weight of the protein composition) can produce no more than 90% (e.g., no more than 80%, 70%, 60%, 50%, 40%, 30%, 20%, or 10%) of the amount of one or more flavor compounds (e.g., soy flavor compounds) produced by cooking a 1% (w/v) suspension of the source protein composition (by dry weight of the source protein composition). For example, when cooked in a flavor broth, a 1% (w/v) suspension of a protein composition (by dry weight of the protein composition) can produce no more than 90% (e.g., no more than 80%, 70%, 60%, 50%, 40%, 30%, 20%, or 10%) of the amount of one or more flavor compounds (e.g., soy flavor compounds) produced by cooking a 1% (w/v) suspension of the source protein composition (by dry weight of the source protein composition). When cooked in a flavor broth (e.g., containing a reducing sugar, a sulfur-containing amino acid, and a heme-containing protein), a 1% (w/v) suspension of a protein composition (by dry weight of the protein composition) can produce at least 5% (e.g., at least 10%, 20%, 30%, 40%, 50%, 60%, 70%, 80%, 90%, 100%, or more) more of the amount of one or more volatile compounds in the meat volatile set produced by cooking a 1% (w/v) suspension of the source protein composition (by dry weight of the source protein composition).

In some embodiments, a set of volatiles can be evaluated for any of the protein compositions as described herein. As defined herein, “volatile set 1” comprises 1-hexanol, 1-octen-3-ol, 1-octen-3-one, 1-pentanol, 2-butanol, 2-decanone, 2-decenal, 2-nonanone, 2,4-decadienal, acetophenone, butanoic acid, 2-pentyl-furan, hexanal, hexanoic acid, octanoic acid, pentanal, and pentanoic acid. In some embodiments, “volatile set 1” consists of 1-hexanol, 1-octen-3-ol, 1-octen-3-one, 1-pentanol, 2-butanol, 2-decanone, 2-decenal, 2-nonanone, 2,4-decadienal, acetophenone, butanoic acid, 2-pentyl-furan, hexanal, hexanoic acid, octanoic acid, pentanal, and pentanoic acid.

As defined herein, “volatile set 2” comprises pentanal, hexanal, 2-pentyl furan, 2,4-decadienal, 2,6-nonadienal, 1-octen-3-ol, 1-octen-3-one, 1-hexanol, 2-decenal, 1-pentanol, acetophenone, 2-decanone, 2-nonanone, 2-butanol, 4-ethylbenzaldehyde, butanoic acid, pentanoic acid, hexanoic acid, and octanoic acid. In some embodiments, “volatile set 2” consists of pentanal, hexanal, 2-pentyl furan, 2,4-decadienal, 2,6-nonadienal, 1-octen-3-ol, 1-octen-3-one, 1-hexanol, 2-decenal, 1-pentanol, acetophenone, 2-decanone, 2-nonanone, 2-butanol, 4-ethylbenzaldehyde, butanoic acid, pentanoic acid, hexanoic acid, and octanoic acid.

As defined herein, “volatile set 3” comprises pentanal, hexanal, 2-pentyl furan, 2,4-decadienal, 2-nonenal, 2,6-nonadienal, 1-octen-3-ol, 1-octen-3-one, 1-hexanol, 2-decenal, 1-pentanol, acetophenone, 2-decanone, 2-nonanone, 2-butanol, 4-ethylbenzaldehyde, butanoic acid, pentanoic acid, hexanoic acid, and octanoic acid. In some embodiments, “volatile set 3” consists of pentanal, hexanal, 2-pentyl furan, 2,4-decadienal, 2-nonenal, 2,6-nonadienal, 1-octen-3-ol, 1-octen-3-one, 1-hexanol, 2-decenal, 1-pentanol, acetophenone, 2-decanone, 2-nonanone, 2-butanol, 4-ethylbenzaldehyde, butanoic acid, pentanoic acid, hexanoic acid, and octanoic acid.

As defined herein, “volatile set 4” comprises butanoic acid, pentanoic acid, hexanoic acid, and octanoic acid. In some embodiments, “volatile set 4” consists of butanoic acid, pentanoic acid, hexanoic acid, and octanoic acid.

As defined herein, “volatile set 5” comprises 1-octen-3-ol, 1-hexanol, and 1-pentanol. In some embodiments, “volatile set 5” consists of 1-octen-3-ol, 1-hexanol, and 1-pentanol.

As defined herein, “volatile set 6” comprises pentanal, hexanal, 2,4-decadienal, 2-nonenal, 2,6-nonadienal, 2-decenal, and 4-ethylbenzaldehyde. In some embodiments, “volatile set 6” consists of pentanal, hexanal, 2,4-decadienal, 2-nonenal, 2,6-nonadienal, 2-decenal, and 4-ethylbenzaldehyde.

As defined herein, “volatile set 7” comprises 2-pentyl furan. In some embodiments, “volatile set 7” consists of 2-pentyl furan.

As defined herein, “volatile set 8” comprises 1-octen-3-one, acetophenone, 2-decanone, 2-nonanone, and 2-butanol. In some embodiments, “volatile set 8” consists of 1-octen-3-one, acetophenone, 2-decanone, 2-nonanone, and 2-butanol.

As defined herein, “volatile set 9” comprises 4-ethylbenzaldehyde, acetophenone, 2-butanol, butanoic acid, 1-pentanol, 2-pentyl furan, pentanal, pentanoic acid, 1-hexanol, hexanal, and hexanoic acid. In some embodiments, “volatile set 9” consists of 4-ethylbenzaldehyde, acetophenone, 2-butanol, butanoic acid, 1-pentanol, 2-pentyl furan, pentanal, pentanoic acid, 1-hexanol, hexanal, and hexanoic acid.

As defined herein, “volatile set 10” comprises 1-octen-3-ol, 1-octen-3-one, octanoic acid, 2,6-nonadienal, 2-nonanone, 2-nonenal, 2,4-decadienal, 2-decanone, and 2-decenal. In some embodiments, “volatile set 10” consists of 1-octen-3-ol, 1-octen-3-one, octanoic acid, 2,6-nonadienal, 2-nonanone, 2-nonenal, 2,4-decadienal, 2-decanone, and 2-decenal.

As defined herein, “meat volatile set” comprises 2,3-butanedione, 2,3-pentanedione, thiazole, 2-acetylthiazole, benzaldehyde, 3-methyl-butanal, 2-methyl-butanal, thiophene, and pyrazine.

A protein composition can, in some embodiments, produce less of one or more volatile compounds that can influence taste when cooked as compared to the amount of the one or more volatile compounds produced by cooking the source protein composition. Without being bound by any particular theory, it is believed that a reduction in volatile content that can influence taste when cooked can allow a protein composition to be suitable to be used in a diverse range of food products. Non-limiting examples of the one or more volatile compounds that can influence taste include the volatile compounds of any of volatile sets 1-10. For example, when cooked in water, a 1% (w/v) suspension of a protein composition (by dry weight of the protein composition) can produce no more than 90% (e.g., no more than 80%, 70%, 60%, 50%, 40%, 30%, 20%, or 10%) of the amount of one or more volatile compounds in a set of volatile compounds produced by cooking 1% (w/v) suspension of the source protein composition (by dry weight of the source protein composition). For example, when cooked in a flavor broth, a 1% (w/v) suspension of a protein composition (by dry weight of the protein composition) can produce no more than 90% (e.g., no more than 80%, 70%, 60%, 50%, 40%, 30%, 20%, or 10%) of the amount of one or more volatile compounds produced by cooking a 1% (w/v) suspension of the source protein composition (by dry weight of the source protein composition). When cooked in a flavor broth, a 1% (w/v) suspension of a protein composition (by dry weight of the protein composition) can produce at least 5% (e.g., at least 10%, 20%, 30%, 40%, 50%, 60%, 70%, 80%, 90%, 100%, or more) more of the amount of one or more volatile compounds in the meat volatile set produced by cooking a 1% (w/v) suspension of the source protein composition (by dry weight of the source protein composition). In some embodiments, a flavor broth comprises one or more (e.g., two or more, three or more, four or more, or five or more) flavor precursor molecules or compounds. The one or more flavor precursors can comprise at least one compound selected from the group consisting of glucose, ribose, cysteine, a cysteine derivative, thiamine, alanine, methionine, lysine, a lysine derivative, glutamic acid, a glutamic acid derivative, IMP, GMP, lactic acid, maltodextrin, creatine, alanine, arginine, asparagine, aspartate, glutamic acid, glutamine, glycine, histidine, isoleucine, leucine, methionine, phenylalanine, proline, threonine, tryptophan, tyrosine, valine, linoleic acid, and mixtures thereof. Suitable flavor precursors can include sugars, sugar alcohols, sugar derivatives, oils (e.g., vegetable oils), free fatty acids, alpha-hydroxy acids, dicarboxylic acids, amino acids and derivatives thereof, nucleosides, nucleotides, vitamins, peptides, protein hydrolysates, extracts, phospholipids, lecithin, and organic molecules. In some embodiments, a set of volatile compounds can comprise a compound in volatile set 1. In some embodiments, a set of volatile compounds can be volatile set 1. In some embodiments, a set of volatile compounds can comprise a compound in volatile set 2. In some embodiments, a set of volatile compounds can be volatile set 2. In some embodiments, a set of volatile compounds can comprise a compound in volatile set 3. In some embodiments, a set of volatile compounds can be volatile set 3. In some embodiments, a set of volatile compounds can comprise a compound in volatile set 4. In some embodiments, a set of volatile compounds can be volatile set 4. In some embodiments, a set of volatile compounds can comprise a compound in volatile set 5. In some embodiments, a set of volatile compounds can be volatile set 5. In some embodiments, a set of volatile compounds can comprise a compound in volatile set 6. In some embodiments, a set of volatile compounds can be volatile set 6. In some embodiments, a set of volatile compounds can comprise a compound in volatile set 7. In some embodiments, a set of volatile compounds can be volatile set 7. In some embodiments, a set of volatile compounds can comprise a compound in volatile set 8. In some embodiments, a set of volatile compounds can be volatile set 8. In some embodiments, a set of volatile compounds can comprise a compound in volatile set 9. In some embodiments, a set of volatile compounds can be volatile set 9. In some embodiments, a set of volatile compounds can comprise a compound in volatile set 10. In some embodiments, a set of volatile compounds can be volatile set 10.

In some embodiments, a protein composition as described herein can include one or more isoflavones in an amount smaller than the amount in the source protein composition. In some cases, a protein composition can have an isoflavone content of less than about 90% (e.g., less than about 80%, 70%, 60%, 50%, 40%, 30%, 20%, or 10%) than the isoflavone content of the source protein composition. In some cases, a protein composition can have a content of daidzein, daidzin, genistein, genistin, glycitein, and glycitin, in total, of less than about 90% (e.g., less than about 80%, 70%, 60%, 50%, 40%, 30%, 20%, or 10%) than the content of daidzein, daidzin, genistein, genistin, glycitein, and glycitin, in total, of the source protein composition. In some cases, a protein composition can have a content of daidzin, genistin, and glycitin, in total, of less than about 90% (e.g., less than about 80%, 70%, 60%, 50%, 40%, 30%, 20%, or 10%) than the content of daidzin, genistin, and glycitin, in total, of the source protein composition. In some cases, a protein composition can have a content of daidzein, genistein, and glycitein, in total, of less than about 90% (e.g., less than about 80%, 70%, 60%, 50%, 40%, 30%, 20%, or 10%) than the content of daidzein, genistein, and glycitein, in total, of the source protein composition. In some cases, the isoflavone content is content of an isoflavone selected from the group consisting of daidzein, daidzin, genistein, genistin, glycitein, glycitin, and any combination thereof. In some cases, a protein composition can have a daidzein content of less than about 90% (e.g., less than about 80%, 70%, 60%, 50%, 40%, 30%, 20%, or 10%) than the daidzein content of the source protein composition. In some cases, a protein composition can have a daidzin content of less than about 90% (e.g., less than about 80%, 70%, 60%, 50%, 40%, 30%, 20%, or 10%) than the daidzin content of the source protein composition. In some cases, a protein composition can have a genistein content of less than about 90% (e.g., less than about 80%, 70%, 60%, 50%, 40%, 30%, 20%, or 10%) than the genistein content of the source protein composition. In some cases, a protein composition can have a genistin content of less than about 90% (e.g., less than about 80%, 70%, 60%, 50%, 40%, 30%, 20%, or 10%) than the genistin content of the source protein composition. In some cases, a protein composition can have a glycitein content of less than about 90% (e.g., less than about 80%, 70%, 60%, 50%, 40%, 30%, 20%, or 10%) than the glycitein content of the source protein composition. In some cases, a protein composition can have a glycitin content of less than about 90% (e.g., less than about 80%, 70%, 60%, 50%, 40%, 30%, 20%, or 10%) than the glycitin content of the source protein composition. In some cases, the isoflavone content is content of an isoflavone selected from the group consisting of formononetin and biochanin A.

In some embodiments, a protein composition as described herein can include one or more phospholipids in an amount smaller than the amount in the source protein composition. In some cases, a protein composition can have a phospholipid content of less than about 90% (e.g., less than about 80%, 70%, 60%, 50%, 40%, 30%, 20%, or 10%) than the phospholipid content of the source protein composition. In some cases, a protein composition can have a phosphatidylcholine-36:4 content of less than about 90% (e.g., less than about 80%, 70%, 60%, 50%, 40%, 30%, 20%, or 10%) than the phosphatidylcholine-36:4 content of the source protein composition. In some embodiments, the phospholipid content is phosphatidylcholine-36:3 content. In some embodiments, the phospholipid content is phosphotidylethanolamine-36:4 content. In some embodiments, the phospholipid content is phosphatidic acid-36:4 content.

In some embodiments, a protein composition as described herein can include one or more saponins in an amount smaller than the amount in the source protein composition. In some cases, a protein composition can have a saponin content of less than about 90% (e.g., less than about 80%, 70%, 60%, 50%, 40%, 30%, 20%, or 10%) of the saponin content of the source protein composition. In some cases, a protein composition can have a soyasaponin content of less than about 90% (e.g., less than about 80%, 70%, 60%, 50%, 40%, 30%, 20%, or 10%) of the soyasaponin content of the source protein composition.

In some embodiments, a protein composition as described herein can include one or more lipids in an amount smaller than the amount in the source protein composition. In some cases, a protein composition can have a lipid content of less than about 90% (e.g., less than about 80%, 70%, 60%, 50%, 40%, 30%, 20%, or 10%) of the lipid content of the source protein composition.

In some embodiments, a protein composition as described herein can include one or more phenolic acids in an amount smaller than the amount in the source protein composition. In some cases, a protein composition can have a phenolic acid content of less than about 90% (e.g., less than about 80%, 70%, 60%, 50%, 40%, 30%, 20%, or 10%) of the phenolic acid content of the source protein composition.

In some embodiments, a protein composition as described herein can include one or more flavor compounds in an amount smaller than the amount in the source protein composition. In some cases, a protein composition can have a flavor compound content of less than about 90% (e.g., less than about 80%, 70%, 60%, 50%, 40%, 30%, 20%, or 10%) of the flavor compound content of the source protein composition. In some embodiments, the flavor compounds are selected from the group consisting of elected from aldehydes, ketones, esters, alcohols, pyrazines, pyranones, acids, sulfur compounds, terpenes, furans, alkanes, alkenes, and combinations thereof.

Flavor can refer to taste and/or aroma. Five basic tastes (i.e., sweet, bitter, sour, salty, and umami or savory) respond primarily to nonvolatile compounds and can be perceived via receptors on the tongue. Aroma refers primarily to volatile compounds, perceived via nasal receptors. Other effects can influence flavor, including but not limited to astringent, dry, rough, metallic, pungent, spicy, cool, and fatty, as well as texture (e.g., smoothness, coarseness, hardness, thickness, slipperiness, viscosity).

Without being bound by any particular theory, it is believed that off-flavors and their precursors may exist as protein-bound complexes in protein sources and/or be generated during harvesting, processing, or storage. Residual phospholipids (PL) and free fatty acids (FFA) in protein compositions may be the precursors of off-flavors. Autoxidation or enzymatic oxidation of PL and FFA during storage may generate off-flavor compounds to unacceptable levels. Further, it is believed that even if off-flavor causing carbonyl compounds are removed from a protein composition, the residual PL and FFA in a protein would continuously generate these carbonyls via autoxidation or enzymatic oxidation during storage.

Volatile compounds that can cause off-flavors can include, but are not limited to, aldehydes, ketones, esters, alcohols, pyrazines, pyranones, acids, sulfur compounds, terpenes, furans, alkanes, and alkenes. Non-limiting examples of off-flavors can include beany, fatty, green, pea, earthy, hay-like, grassy, rancid, leafy, cardboard, acrid, pungent, medicinal, metallic, and brothy. Nonvolatile compounds can also cause off-flavors. For example, isoflavones can cause bitter off-flavors, saponins can cause astringent off-flavors, and phenolic acids, peptides, or amino acids can cause metallic off-flavors.

The methods provided herein can also be used to prepare a detoxified protein composition. As used herein, a “detoxified protein composition” refers to a protein composition prepared from a source protein composition that is otherwise unsuitable for human consumption (e.g., due to the presence or amount of one or more toxins), wherein the protein composition has one or more toxins removed or reduced in amount as compared to the source protein composition such that the detoxified protein composition is suitable for human consumption.

In some embodiments, the method for making a detoxified protein composition comprises: (a) adding an aqueous solution to a source protein composition to form a solution of solubilized protein; (b) optionally removing solids from the solution of solubilized protein; (c) adding an organic solvent to the solution of solubilized protein to form a solid phase and a liquid phase, and (d) separating the solid phase from the liquid phase to form a detoxified protein composition, wherein the detoxified protein composition comprises a plurality of plant proteins, fungal proteins, algal proteins, bacterial proteins, protozoan proteins, invertebrate (e.g., insect and/or arachnid) proteins, and wherein the source protein composition is not suitable for human consumption.

In some such embodiments, the source protein composition comprises one or more toxins in an amount sufficient to harm a human being. For example, the source protein composition can be a cottonwood source protein composition. In some embodiments, the source protein composition comprises a toxic phenolic compound, such as gossypol. For example, the source protein composition can include gossypol in an amount of more than 450 ppm. Accordingly, in some embodiments, the detoxified protein composition comprises gossypol in an amount of less than 450 ppm (e.g., less than about 300 ppm; less than about 100 ppm; less than about 50 ppm; less than about 10 ppm, less than about 5 ppm, or less than about 2 ppm). In some embodiments, a protein composition as described herein can include one or more toxins in an amount smaller than the amount in the source protein composition. In some cases, a protein composition can have a toxin content of less than about 90% (e.g., less than about 70%, 50%, 30%, or 10%) of the toxin content of the source protein composition. Non-limiting examples of toxins include gossypol (for example, in cottonwood), vicine or convicine (for example, in faba beans), cyanogenic glycosides (for example, in cassava or bamboo), glucosinolates (for example, in cruciferous vegetables), and glycoalkaloids (for example, in potato and bittersweet nightshade).

Various methods can be used to determine the amount of the one or more toxins in the source protein composition or the detoxified protein composition (e.g., spectrophotometry, HPLC, enzyme-linked immunosorbent assay (ELISA)). In some embodiments, a toxin can also contribute to color of a protein composition, and its removal can result in the protein composition being lower in color. For example, gossypol typically has a green-yellow color.

Also provided herein are methods for extracting small molecules from a protein source composition. In some embodiments, the methods include: (a) adding an aqueous solution to a source protein composition to form a solution of solubilized protein; (b) optionally removing solids from the solution of solubilized protein; (c) adding an organic solvent to the solution of solubilized protein to form a solid phase and a liquid phase, and (d) separating the solid phase from the liquid phase to form a solution enriched in small molecules. For example, the source protein composition can be a soy source protein composition. In some such embodiments, the small molecules to be extracted can include one or more isoflavones. For example, the one or more isoflavones can include genistein and daidzein. The small molecules to be extracted can include isoflavones, pigments (e.g., chlorophylls, anthocyanins, carotenoids, and betaiains), flavor compounds (e.g., soy flavor compounds), saponin, toxins (e.g., gossypol), natural products (e.g., plant natural products, pharmacologically active natural products), metabolites (e.g., primary and/or secondary metabolites), and/or phospholipids (e.g., lecithin). For example, isoflavones and saponins may have medical or nutritional uses. Lecithin may be used as an emulsifier, for example in food products, or as a choline-rich nutrient source. The small molecules can have molecular weights up to 900 daltons (e.g., up to 800, up to 700, up to 600, or up to 500 daltons). In some embodiments, the extracted small molecules can be useful as supplements. In some embodiments, the extracted small molecules can be useful as food ingredients (e.g., food colorants or flavor compounds). In some embodiments, the extracted small molecules can be useful as chemical precursors for industrial synthesis (e.g., pharmaceutical synthesis). ). As non limiting examples, isoflavones have been suggested to lower the risk of breast cancer, to prevent or inhibit the progression of prostate cancer, and to reduce menopause symptoms; soy isoflavones are sold as nutrient supplement; saponins are thought to decrease blood lipids, lower cancer risks, and lower blood glucose response, and are also sold as nutrient supplement; and soy lecithin (phospholipids) are sold as a food emulsifier, and soy lecithin is rich in choline which is an essential nutrient for human and animals.

Also provided herein are protein compositions. In some embodiments, a protein composition can be produced by any of the methods described herein.

In some cases, protein compositions can be compared to commercial protein products. Non-limiting examples of commercial protein products are protein concentrates and protein isolates. In some embodiments, a comparison can be based on the agricultural source of the protein in the protein composition. For example, a soy protein composition as described herein can be compared to a commercial soy protein product. In some embodiments, a comparison can be based on the protein type. For example, a protein composition that is a protein isolate as described herein can be compared to a commercial protein isolate product, while a protein composition that is a protein concentrate as described herein can be compared to a commercial protein concentrate product. In some embodiments, a comparison can be made on the basis of both the agricultural source of the protein in the protein composition and the protein type. For example, protein composition that is a canola protein concentrate as described herein can be compared to a commercial canola protein concentrate. In some embodiments, a commercial protein product can be a soy protein concentrate. In some embodiments, a commercial protein product can be a soy protein isolate.

Examples of commercial protein products include, without limitation, commercially available soy protein isolate, commercially available soy protein isolate, commercially available pea protein isolate, and commercially available canola protein isolate. In some embodiments, a protein composition as provided herein can be a protein concentrate (e.g., a soy protein concentrate), and the commercial protein product can be a protein concentrate (e.g., a soy protein concentrate). In some embodiments, a protein composition as provided herein can be a protein isolate (e.g., a soy protein isolate), and the commercial protein product can be a protein isolate (e.g., a soy protein isolate).

In some embodiments, provided herein is a protein composition comprising at least 50% by dry weight of a plurality of plant proteins, fungal proteins, algal proteins, bacterial proteins, protozoan proteins, invertebrate proteins, or a combination thereof, wherein the protein composition is a low color protein composition. In some embodiments, the protein composition is a low color protein composition.

In some embodiments, provided herein is a protein composition comprising at least 50% by dry weight of a plurality of plant proteins, fungal proteins, algal proteins, bacterial proteins, protozoan proteins, invertebrate proteins, or a combination thereof; and less than 1.0% by dry weight of lipids.

Protein compositions as described herein typically have a protein content of at least 50% (e.g., at least 55%, 60%, 65%, 70%, 75%, 80%, 85%, 90%, 95%, or 99%) by dry weight of the protein composition. In some embodiments, a protein composition as described herein can have a protein content of at least about 90% (e.g., at least 90.5%, 91%, 91.5%, 92%. 92.5%, 93%, 93.5%, 94%, 94.5%, 95%, 97%, or 99%) by dry weight of the protein composition. In some embodiments, a protein composition as described herein can have a protein content of about 60% to about 80% (e.g., about 65% to about 75%) by dry weight of the protein composition. The protein content of a protein composition may vary based on whether the protein composition is a protein concentrate or a protein isolate. In some cases, a protein concentrate can have a protein content of about 55% to about 75% (e.g., about 55% to about 70%, about 55% to about 65%, about 55% to about 60%, about 60% to about 75%, about 65% to about 75%, about 70% to about 75%) by dry weight of the protein composition. In some cases, a protein isolate can have a protein content of about 80% to about 99% (e.g., about 80% to about 95%, about 80% to about 95%, about 80% to about 85%, about 85% to about 99%, about 90% to about 99%, or about 95% to about 99%) by dry weight of the protein composition. In some cases, a protein isolate can have less than about 8% (e.g., less than about 7%, 6%, 5%, 4%, 3%, 2%, or 1%) by dry weight carbohydrates (e.g., insoluble carbohydrates). In some cases, a protein concentrate can have at least about 8% (e.g., at least about 9%, 10%, 11%, 12%, 13%, 14%, 15%, 16%, 17%, 18%, 19%, 20%, or more) by dry weight carbohydrates (e.g., insoluble carbohydrates).

The proteins in a protein composition as described herein can be any appropriate proteins. In some embodiments, the plurality of plant proteins, fungal proteins, algal proteins, bacterial proteins, protozoan proteins, invertebrate proteins, or the combination thereof comprises at least 90% plant proteins. In some embodiments, the plurality of plant proteins, fungal proteins, algal proteins, bacterial proteins, protozoan proteins, invertebrate proteins, or the combination thereof comprises at least 90% legume proteins. In some embodiments, the plurality of plant proteins, fungal proteins, algal proteins, bacterial proteins, protozoan proteins, invertebrate proteins, or the combination thereof comprises at least 90% pulse proteins. In some embodiments, the plurality of plant proteins, fungal proteins, algal proteins, bacterial proteins, protozoan proteins, invertebrate proteins, or the combination thereof comprises at least 90% soy proteins. In some embodiments, the plurality of plant proteins, fungal proteins, algal proteins, bacterial proteins, protozoan proteins, invertebrate proteins, or the combination thereof comprises at least 90% fungal proteins. In some embodiments, the plurality of plant proteins, fungal proteins, algal proteins, bacterial proteins, protozoan proteins, invertebrate proteins, or the combination thereof comprises at least 90% yeast proteins. In some embodiments, the plurality of plant proteins, fungal proteins, algal proteins, bacterial proteins, protozoan proteins, invertebrate proteins, or the combination thereof comprises at least 90% algal proteins.

Protein compositions can be produced using any appropriate starting material, such as any of those described herein, or a mixture of any thereof. Thus, a protein composition as described herein can include a plurality of plant proteins, fungal proteins, algal proteins, bacterial proteins, invertebrate (e.g., insect and/or arachnid) proteins, or a combination thereof.

In some embodiments, protein compositions as described herein include proteins that are substantially aggregated, denatured, or both. Aggregation and/or denaturation can be determined by any appropriate method. In some cases, aggregation can be measured by average particle size (e.g., using dynamic light scattering (DLS)). In some embodiments, a protein composition as described herein can have an average particle size of about 1 µm to about 40 µm (e.g., about 5 to about 40 µm, about 10 to about 40 µm, about 20 to about 40 µm, about 30 to about 40 µm, about 1 to about 5 µm, about 1 to about 10 µm, about 1 to about 20 µm, about 1 to about 30 µm, about 10 to about 30 µm, or about 20 to about 30 µm) in the largest dimension. In some embodiments, the size and shape of the particle size distribution can be related to the conditions in which the protein composition was precipitated. In some embodiments, particles in a protein composition as described herein can have a zeta potential of about -1.5 to about -4.5 mV. In some embodiments, the particle charge can be related to the conditions in which the protein composition was precipitated. In some cases, the surface hydrophobicity and protein solubility of a protein composition can be tunable. In some cases, denaturation or unfolding can be measured by circular dichroism spectroscopy, differential scanning calorimetry, or a fluorescent dye assay in which the dye binds to hydrophobic regions exposed during protein unfolding. In some cases, denaturation can be correlated to a loss of temperature-dependent change in one or more mechanical properties (e.g., storage modulus, loss modulus, and/or viscosity) over a temperature range (e.g., heating from 25° C. to 95° C., heating from 40° C. to 95° C., heating from 60° C. to 95° C., or heating from 80° C. to 90° C.).

In some embodiments, a protein composition as described herein has a protein dispersibility index of at least about 5 (e.g., at least about 10 or at least about 15). In some embodiments, a protein composition as described herein has a sodium level up to about 1 %w/w (e.g., up to about 0.5, up to about 0.1, up to about 0.05, up to about 0.01, or up to about 0.005 %w/w).

In some embodiments, a protein composition as described herein can have a solubility of at least 5% (e.g., at least 10%, at least 15%, at least 20%, at least 25%, or at least 30%) in an aqueous solution (e.g., water). In some embodiments, the aqueous solution has a pH of about 6.0 to about 8.0, of about 6.5 to about 7.5, of about 7.0 to about 8.0, of about 7.0, or of about 8.0. In some embodiments, the aqueous solution can include a buffer.

In some embodiments, a protein composition as described herein can exhibit a temperature-dependent change in one or more mechanical properties (e.g., storage modulus, loss modulus, and/or viscosity) over a temperature range (e.g., heating from 25° C. to 95° C., heating from 40° C. to 95° C., heating from 60° C. to 95° C., or heating from 80° C. to 90° C.). In some embodiments, the temperature-dependent change is at least 5-fold (e.g., at least 10-fold, at least 100-fold, at least 500-fold, or at least 1,000-fold) in magnitude. In some embodiments, the temperature-dependent change is substantially irreversible (e.g., upon cooling over the same temperature range, the magnitude of the change is up to 25%, up to 20%, up to 15%, up to 10%, up to 5%, up to 1%, up to 0.5%, or up to 0.1% the magnitude of the change observed upon heating). In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 90° C. In some embodiments, the storage modulus and/or loss modulus reach a value of at least 1,000 Pa (e.g., at least 2,000 Pa, at least 3,000 Pa, at least 4,000 Pa, at least 5,000 Pa, at least 6,000 Pa, at least 7,000 Pa, at least 8,000 Pa, at least 9,000 Pa, or at least 10,000 Pa) at 95° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 90° C. In some embodiments, the viscosity reaches a value of at least 1,000 Pa·s (e.g., at least 2,000 Pa·s, at least 3,000 Pa·s, at least 4,000 Pa·s, at least 5,000 Pa·s, at least 6,000 Pa·s, at least 7,000 Pa·s, at least 8,000 Pa·s, at least 9,000 Pa·s, or at least 10,000 Pa·s) at 95° C.

Protein compositions as described herein can include components other than protein. In some cases, protein compositions as described herein can include carbohydrates (e.g., insoluble carbohydrates), lipids (e.g., a fatty acid, a wax, a sterol, a monoglyceride, a diglyceride, a triglyceride, a sphingolipid, a phospholipid, or a combination thereof), saponins, or a combination thereof. In some embodiments, a protein composition as described herein can include lipids in an amount less than about 1.5% (e.g., less than about 1.3%, 1.2%, 1.1%, 1.0%, 0.9%, 0.8%, 0.7%, 0.6%, 0.5%, 0.4%, 0.3%, 0.2%, 0.1%, or less) by dry weight of the protein composition. In some embodiments, a protein composition as described herein can include lipids in an amount less than about 1.2% by dry weight of the protein composition. In some embodiments, a protein composition can include lipids in an amount of less than about 1.0% by dry weight of the protein composition. In some embodiments, a protein composition can include lipids in an amount of less than about 0.8% by dry weight of the protein composition. In some embodiments, a protein composition can include lipids in an amount of less than about 0.7% by dry weight of the protein composition. In some embodiments, a protein composition can include lipids in an amount of less than about 0.6% by dry weight of the protein composition. In some embodiments, a protein composition can include lipids in an amount of less than about 0.5% by dry weight of the protein composition. In some embodiments, a protein composition can include lipids in an amount of less than about 0.4% by dry weight of the protein composition. In some embodiments, a protein composition as described herein can include lipids in an amount less than about 0.5% by dry weight of the protein composition. In some embodiments, a protein composition as described herein can include phospholipids in an amount less than about 0.5% (e.g., less than about 0.4%, 0.3%, 0.2%, or 0.1%) by dry weight of the protein composition. In some cases, phosphatidylcholine 36:4 can be used as a surrogate measurement for total phospholipids. In some embodiments, a protein composition as described herein can have a reduced amount of one or more of: a fatty acid, a wax, a sterol, a monoglyceride, a diglyceride, a triglyceride, or a phospholipid as compared to the source of the protein in the protein composition. In some embodiments, a protein composition as described herein can have a reduced amount (e.g., reduced by at least 5%, 10%, 20%, 25%, 30%, 40%, 50%, 60%, 70%, 80%, 90%, 95%, or more) of a phospholipid (e.g., a phosphatidylcholine (e.g., phosphatidylcholine-36:4, phosphatidylcholine-34:2, phosphatidylcholine-36:3), a phosphotidylethanolamine (e.g., phosphotidylethanolamine-36:4), a glycerophospholipid, a phosphatidic acid (e.g., phosphatidic acid-36:4), a phosphatidylserine, a phosphoinositide, or a combination thereof) as compared to the source of the protein in the protein composition (or, e.g., the source protein composition from which the protein composition was made).

Saponins can cause foaming of solutions. In some embodiments, a protein composition as described herein can have a lower saponin content than the saponin content of the source of the protein in the composition (or, e.g., the source protein composition from which the protein composition was made). In some embodiments, a protein composition as described herein can have a saponin content of less than 90% (e.g., less than 80%, 70%, 60%, 50%, 40%, 30%, 20%, 10%, or less) of the saponin content of the source of the protein in the composition (or, e.g., the source protein composition from which the protein composition was made). In some embodiments, a protein isolate as described herein can have a lower saponin content than the saponin content of a commercial protein isolate. In some embodiments, a protein isolate as described herein can have a saponin content of less than 90% (e.g., less than 80%, 70%, 60%, 50%, 40%, 30%, 20%, 10%, or less) of the saponin content of a commercial protein isolate.

In some embodiments, a protein composition as described herein can include one or more isoflavones. In some cases, a protein composition can have an isoflavone content of less than about 500 ppm (e.g., less than about 400 ppm, 300 ppm, 250 ppm, 200 ppm, 150 ppm, 125 ppm, 100 ppm, 75 ppm, or 50 ppm). In some cases, the isoflavone content is content of daidzein, daidzin, genistein, genistin, glycitein, and glycitin, in total. In some cases, a protein composition can have a content of daidzein, daidzin, genistein, genistin, glycitein, and glycitin, in total, of less than about 250 ppm (e.g., less than about 200 ppm, 150 ppm, 125 ppm, 100 ppm, 75 ppm, or 50 ppm). In some cases, the isoflavone content is content of daidzin, genistin, and glycitin, in total. In some embodiments, a protein composition can have a content of daidzin, genistin, and glycitin, in total, of less than about 200 ppm (e.g., less than about 150 ppm, 100 ppm, or 75 ppm). In some cases, the isoflavone content is content of daidzein, genistein, and glycitein, in total. In some embodiments, a protein composition can have a content of daidzein, genistein, and glycitein, in total, of less than about 50 ppm (e.g., less than about 30 ppm, 20 ppm, or 10 ppm). In some cases, the isoflavone content is content of an isoflavone selected from the group consisting of daidzein, daidzin, genistein, genistin, glycitein, glycitin, and any combination thereof. In some embodiments, a protein composition can have a content of daidzein of less than about 100 ppm (e.g., less than about 75 ppm, 50 ppm, 30 ppm, 20 ppm, 10 ppm, 5 ppm, or 3 ppm). In some embodiments, a protein composition can have a content of daidzin of less than about 100 ppm (e.g., less than about 75 ppm, 50 ppm, 30 ppm, or 10 ppm). In some embodiments, a protein composition can have a content of genistein of less than about 100 ppm (e.g., less than about 75 ppm, 50 ppm, 20 ppm, 10 ppm, 5 ppm, 3 ppm or 1 ppm). In some embodiments, a protein composition can have a content of genistin of less than about 300 ppm (e.g., less than about 200 ppm, 100 ppm, 75 ppm, 50 ppm, or 30 ppm). In some embodiments, a protein composition can have a content of glycitein of less than about 30 ppm (e.g., less than about 20 ppm, 10 ppm, 5 ppm, 3 ppm, or 1 ppm). In some embodiments, a protein composition can have a content of glycitin of less than about 30 ppm (e.g., less than about 20 ppm, 10 ppm, or 5 ppm). In some cases, the isoflavone content is content of an isoflavone selected from the group consisting of formononetin and biochanin A.

In some embodiments, a protein composition as described herein can include one or more phospholipids. In some cases, a protein composition can have a phospholipid content of less than about 1,000 ppm (e.g., less than about 750 ppm, 500 ppm, 250 ppm, 100 ppm, 50 ppm, 25 ppm, 10 ppm, 5 ppm, 2 ppm, or 1 ppm). In some embodiments, the phospholipid content is phosphatidylcholine-36:4 content. In some embodiments, a protein composition can have a phosphatidylcholine-36:4 content of less than about 500 ppm (e.g., less than about 250 ppm, 100 ppm, 50 ppm, 25 ppm, 10 ppm, 5 ppm, 2 ppm, or 1 ppm). In some embodiments, the phospholipid content is phosphatidylcholine-34:2 content. In some embodiments, a protein composition can have a phosphatidylcholine-34:2 content of less than about 750 ppm (e.g., less than about 500 ppm, 250 ppm, 100 ppm, 50 ppm, 25 ppm, 10 ppm, 5 ppm, 2 ppm, or 1 ppm). In some embodiments, the phospholipid content is phosphatidylcholine-36:3 content. In some embodiments, the phospholipid content is phosphotidylethanolamine-36:4 content. In some embodiments, the phospholipid content is phosphatidic acid-36:4 content.

In some embodiments, a protein composition as described herein can include one or more saponins. In some cases, a protein composition can have a saponin content of less than about 1000 ppm (e.g., less than about 750 ppm, 500 ppm, 250 ppm, 100 ppm, 75 ppm, 50 ppm, or 25 ppm). In some cases, the saponin content is soyasaponin content. In some cases, a protein composition can have a soyasaponin content of less than about 1000 ppm (e.g., less than about 750 ppm, 500 ppm, 250 ppm, 100 ppm, 75 ppm, 50 ppm, or 25 ppm).

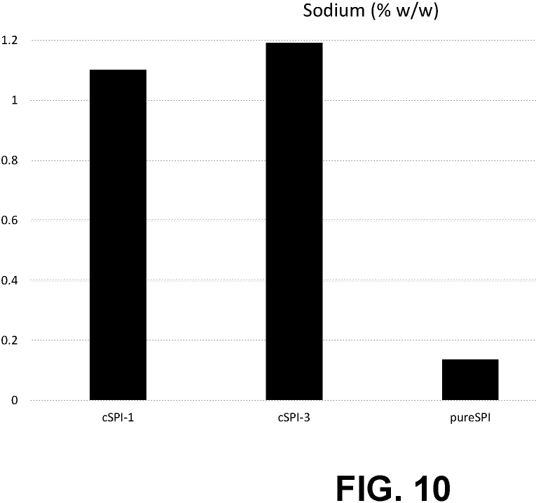

In some embodiments, protein compositions as described herein can include sodium. Without being bound by any particular theory, it is believed that various commercial processes, for example, isoelectric point precipitation, can introduce sodium into a protein product. In some embodiments, a protein composition as described herein can have less sodium than a commercial protein product. Exemplary sodium content is shown in FIG. 10.

In some embodiments, a protein composition (e.g., a protein concentrate) as described herein can have a sodium content of about 0.0005% to about 0.01% (w/w) (e.g., about 0.0005% to about 0.001%, about 0.0005% to about 0.002%, about 0.0005% to about 0.003%, about 0.0005% to about 0.004%, about 0.0005% to about 0.005%, about 0.0005% to about 0.007%, about 0.0005% to about 0.0009%, about 0.001% to about 0.01%, about 0.002% to about 0.01%, about 0.003% to about 0.01%, about 0.004% to about 0.01%, about 0.005% to about 0.01%, about 0.007% to about 0.01%, or about 0.009% to about 0.01% (w/w)). In some embodiments, a protein composition (e.g., a protein isolate) as described herein can have a sodium content of about 0.05% to about 0.3% (w/w) (e.g., about 0.05% to about 0.1%, about 0.05% to about 0.2%, about 0.1% to about 0.2%, about 0.1% to about 0.3%, or about 0.2% to about 0.3% (w/w). In some cases, a protein composition can have a sodium content of less than about 1% (w/w) (e.g., less than about 0.9%, 0.8%, 0.7%, 0.6%, 0.5%, 0.4%, 0.3%, 0.2%, or 0.1% (w/w)).

A protein composition as described herein can include non-organic content (sometimes referred to as “ash” in an analysis). The non-organic content can include salts, such as sodium salts. In some embodiments, a protein composition as described herein can include non-organic content in an amount of about 4% to about 8% (e.g., about 4% to about 7%, about 4% to about 6%, about 4% to about 5%, about 5% to about 8%, about 6% to about 8%, about 7% to about 8%, or about 5% to about 6%) by dry weight of the protein composition.

In some cases, protein compositions as described herein can have parameters that make them well suited to being ingredients in food. For example, protein compositions as described herein can be one or more of: low color, low flavor, and detoxified.

The color of a protein composition can be determined by any appropriate assay. In some cases, the relative luminance of a protein composition can be evaluated, where an internal white control is rated 100, and an internal black control is rated 0. In some embodiments, a protein composition as described herein can have a luminance of at least 85 (e.g., at least 86, 87, 88, 89, 90, 91, 92, or more) on this relative scale. In some embodiments, a protein composition as described herein can have a luminance of at least 85 (e.g., at least 85.5, 86, 86.5, 87, 87.5, 88, 88.5, 89, 89.5, 90, 90.5, 91, 91.5, 92, or more) on this relative scale. In some cases, the chroma (a unitless measure presented herein on a scale from 0-100) of a protein composition can be evaluated, e.g., using a chroma meter or colorimeter. In some embodiments, a protein composition as described herein can have a chroma value of less than 15 (e.g., less than 14, 13, 12, 11, 10, 9, 8, 7, 6, 5, or lower). In some embodiments, a protein composition as described herein can have a chroma value of less than 15 (e.g., less than 14.5, 14, 13.5, 13, 12.5, 12, 11.5, 11, 10.5, 10, 9.5, 9, 8.5, 8, 7.5, 7, 6.5, 6, 5.5, 5, or lower). In some embodiments, a low color protein composition can have a luminance of at least about 85 (e.g., at least 86, 87, 88, 89, 90, 91, 92, or more), a chroma value less than of less than 15 (e.g., less than 14, 13, 12, 11, 10, 9, 8, 7, 6, 5, or lower), or both. In some embodiments, a low color protein composition can have a luminance of at least about 85 (e.g., at least 85.5, 86, 86.5, 87, 87.5, 88, 88.5, 89, 89.5, 90, 90.5, 91, 91.5, 92, or more), a chroma value less than of less than 15 (e.g., less than 14.5, 14, 13.5, 13, 12.5, 12, 11.5, 11, 10.5, 10, 9.5, 9, 8.5, 8, 7.5, 7, 6.5, 6, 5.5, 5, or lower), or both.

The flavor of a protein composition (or, e.g., a source of the protein in a protein composition, a source protein composition, or a commercial protein product) can be determined using any appropriate method. In some cases, a protein composition, a source of the protein in the composition, a source protein composition, or a commercial protein product can be ground into a powder before flavor analysis. Grinding into a powder can performed by any appropriate method. For example, a cryogenic mill (e.g., a SPEX Freezer Mill) or a blender (e.g., a high -performance blender, such as a Vitamix brand blender, in which case temperature is optionally monitored) can be used. In some embodiments, the amount of one or more volatile compounds produced by the protein composition (or a source of the protein in a protein composition, a source protein composition, or a commercial protein product, e.g., for the purpose of comparison to a protein composition provided herein) (e.g., as a 1% (w/v) suspension) can be evaluated without heating (e.g., without cooking). In some embodiments, the amount of one or more volatile compounds produced by cooking the protein composition (or a source of the protein in a protein composition, a source protein composition, or a commercial protein product, e.g., for the purpose of comparison to a protein composition provided herein) (e.g., as a 1% (w/v) suspension) can be evaluated. In some embodiments, a protein composition (or a source of the protein in a protein composition, a source protein composition, or a commercial protein product, e.g., for the purpose of comparison to a protein composition provided herein) can be cooked in water (e.g., tap water). In some embodiments, a protein composition (or a source of the protein in a protein composition, a source protein composition, or a commercial protein product, e.g., for the purpose of comparison to a protein composition provided herein) can be cooked in a flavor broth. In some embodiments, a flavor broth comprises one or more (e.g., two or more, three or more, four or more, or five or more) flavor precursor molecules or compounds. The one or more flavor precursors can comprise at least one compound selected from the group consisting of glucose, ribose, cysteine, a cysteine derivative, thiamine, alanine, methionine, lysine, a lysine derivative, glutamic acid, a glutamic acid derivative, IMP, GMP, lactic acid, maltodextrin, creatine, alanine, arginine, asparagine, aspartate, glutamic acid, glutamine, glycine, histidine, isoleucine, leucine, methionine, phenylalanine, proline, threonine, tryptophan, tyrosine, valine, linoleic acid, and mixtures thereof. Suitable flavor precursors can include sugars, sugar alcohols, sugar derivatives, oils (e.g., vegetable oils), free fatty acids, alpha-hydroxy acids, dicarboxylic acids, amino acids and derivatives thereof, nucleosides, nucleotides, vitamins, peptides, protein hydrolysates, extracts, phospholipids, lecithin, and organic molecules. In some embodiments, a flavor broth can include a reducing sugar, a sulfur-containing amino acid, and a heme-containing protein. In some cases, a protein isolate as described herein can produce a smaller amount of one or more volatile compounds when cooked as compared to the amount of the one or more volatiles produced by cooking the source of the protein in the protein composition (or a source protein composition, or a commercial protein isolate, e.g., for the purpose of comparison to a protein isolate provided herein). In some cases, a protein isolate as described herein can produce a greater amount of one or more volatiles in the meat volatile set when cooked in a flavor broth as compared to the amount of one or more volatiles in the meat volatile set produced by cooking the source protein composition in the protein composition (or a source protein composition, or a commercial protein isolate, e.g., for the purpose of comparison to a protein isolate provided herein). In some cases wherein when cooked in a solution comprising a reducing sugar, a sulfur-containing amino acid, and a heme-containing protein, a 1% (w/v) of the protein composition produces one or more volatile compounds associated with the aroma and/or taste of meat. In some embodiments, at least one of the one or more volatile compounds associated with the aroma and/or taste of meat is produced in a smaller amount when the reducing sugar, the sulfur-containing amino acid, and the heme-containing protein are cooked in the absence of the protein composition. In some embodiments, at least one of the one or more volatile compounds associated with the aroma and/or taste of meat is not produced when the reducing sugar, the sulfur-containing amino acid, and the heme-containing protein are cooked in the absence of the protein composition. In some embodiments, the one or more volatile compounds associated with the aroma and/or taste of meat comprise at least one compound selected from the group consisting of 2,3-butanedione, 2,3-pentanedione, thiazole, 2-acetylthiazole, benzaldehyde, 3-methyl-butanal, 2-methyl-butanal, thiophene, pyrazine, and combinations thereof. In some cases, “cooking” can mean 3 ml of a sample is sealed in a 20-ml GC glass vial and cooked in a 150 C heating block for 3 minutes with vigorous agitations (e.g., 750 rpm). In some cases, volatile compounds can be evaluated using gas chromatography mass spectrometry (GCMS). For example, the volatile compounds in the headspace of a 1% (w/v) suspension (cooked or not cooked) can be extracted using solid-phase microextraction (SPME) fiber (e.g., DVB/CAR/PDMS) at 50° C. Volatile compounds can be separated on a chromatography column, e.g., on a capillary wax column with temperature ramp from 35° C. to 255° C. Mass spectra can be collected, e.g., at 10 Hz with mass range from 20 to 500.

In some cases, the one or more volatiles may be indicative of the source of the protein in the protein composition. For example, if the source of the protein in the protein composition is soy, a reduction in the amount of one or more soy flavor compounds can be observed, in some cases. Non-limiting examples of compounds that are flavor compounds (e.g., soy flavor compounds) include hexanal, pentanal, 2-pentylfuran, 1-octen-3-ol, 1-octen-3-one, 1-hexanol, (E)-2-nonenal, (E,Z)-2,6-nonadienal, (E,E)-2,4-decadienal, and combinations thereof. Flavor compounds (e.g., soy flavor compounds) can include isoflavones or saponins. Other examples of soy flavor compounds may be found in the literature, for example, in Kao, Jian-Wen, Earl G. Hammond, and Pamela J. White. “Volatile compounds produced during deodorization of soybean oil and their flavor significance.” Journal of the American Oil Chemists’ Society 75.12 (1998): 1103-1107; Solina, Marica, et al. “Volatile aroma components of soy protein isolate and acid-hydrolysed vegetable protein.” Food chemistry 90.4 (2005): 861-873.; Irwin, Anthony J., John D. Everard, and Robert J. Micketts. “Identification of Flavor-Active Volatiles in Soy Protein Isolate via Gas Chromatography Olfactometry.” Chemistry, Texture, and Flavor of Soy. American Chemical Society, 2010. 389-400.; or Lei, Q., and W. L. Boatright. “Compounds contributing to the odor of aqueous slurries of soy protein concentrate.” Journal of food science 66.9 (2001): 1306-1310., Ramasamy Ravi, Ali Taheri, Durga Khandekar, and Reneth Millas. “Rapid Profiling of Soybean Aromatic Compounds Using Electronic Nose.” Biosensors 2019, 9(2), 66, each of which is herein incorporated by reference in its entirety. Other examples of flavor compounds may be found in the literature, for example, in Wibke S. U. Roland et al. “Flavor Aspects of Pulse Ingredients.” Cereal Chemistry 2017, 94(1), 58-65, which is herein incorporated by reference in its entirety.

In some embodiments, when cooked (e.g., as a 1% (w/v) suspension) in water, a protein composition as described herein can produce a lesser amount (e.g., no more than 90% (e.g., no more than 80%, 70%, 60%, 50%, 40%, 30%, 20%, or 10%)) of one or more compounds in a set of volatile compounds than the amount of the one or more compounds in the set of volatile compounds produced by cooking the source of the protein in the protein composition (or, e.g., the source protein composition from which the protein composition was made) (e.g., as a 1% (w/v) suspension) in water. In some embodiments, when cooked (e.g., as a 1% (w/v) suspension) in a flavor broth, a protein composition as described herein can produce a lesser amount (e.g., no more than 90% (e.g., no more than 80%, 70%, 60%, 50%, 40%, 30%, 20%, or 10%)) of one or more compounds in a set of volatile compounds than the amount of the one or more compounds in the set of volatile compounds produced by cooking the source of the protein in the protein composition (or, e.g., the source protein composition from which the protein composition was made) (e.g., as a 1% (w/v) suspension) in the flavor broth.

In some embodiments, when cooked (e.g., as a 1% (w/v) suspension) in water, a protein isolate as described herein can produce a lesser amount (e.g., no more than 90% (e.g., no more than 80%, 70%, 60%, 50%, 40%, 30%, 20%, or 10%)) of one or more compounds in a set of volatile compounds than the amount of the one or more compounds in the set of volatile compounds produced by cooking a commercial protein isolate (e.g., as a 1% (w/v) suspension) in water. In some embodiments, when cooked (e.g., as a 1% (w/v) suspension) in a flavor broth, a protein isolate as described herein can produce a lesser amount (e.g., no more than 90% (e.g., no more than 80%, 70%, 60%, 50%, 40%, 30%, 20%, or 10%)) of one or more compounds in a set of volatile compounds than the amount of the one or more compounds in the set of volatile compounds produced by cooking a commercial protein isolate (e.g., as a 1% (w/v) suspension) in the flavor broth.

In some embodiments, a set of volatile compounds can comprise a compound volatile in set 1. In some embodiments, a set of volatile compounds can be volatile set 1. In some embodiments, a set of volatile compounds can comprise a compound in volatile set 2. In some embodiments, a set of volatile compounds can be volatile set 2. In some embodiments, a set of volatile compounds can comprise a compound in volatile set 3. In some embodiments, a set of volatile compounds can be volatile set 3. In some embodiments, a set of volatile compounds can comprise a compound in volatile set 4. In some embodiments, a set of volatile compounds can be volatile set 4. In some embodiments, a set of volatile compounds can comprise a compound in volatile set 5. In some embodiments, a set of volatile compounds can be volatile set 5. In some embodiments, a set of volatile compounds can comprise a compound in volatile set 6. In some embodiments, a set of volatile compounds can be volatile set 6. In some embodiments, a set of volatile compounds can comprise a compound in volatile set 7. In some embodiments, a set of volatile compounds can be volatile set 7. In some embodiments, a set of volatile compounds can comprise a compound in volatile set 8. In some embodiments, a set of volatile compounds can be volatile set 8. In some embodiments, a set of volatile compounds can comprise a compound in volatile set 9. In some embodiments, a set of volatile compounds can be volatile set 9. In some embodiments, a set of volatile compounds can comprise a compound in volatile set 10. In some embodiments, a set of volatile compounds can be volatile set 10.

A commercial protein product can be any appropriate commercial protein product, such as a commercial soy protein product (e.g., soy protein isolate).

In some embodiments, a protein composition provided herein, or a food product comprising a protein composition as provided herein can be favorably evaluated by a panel of trained tasters. In some embodiments, when assessed by a trained descriptive panel using the Spectrum method, a protein composition as described herein is described as having low intensity of one or more of: oxidized/rancid flavor, cardboard flavor, astringent flavor, bitter flavor, vegetable complex flavor, and sweet fermented flavor. In some embodiments, when assessed by a trained descriptive panel using the Spectrum method, a protein composition as described herein is described as having low intensity of one or more of: beany flavor, fatty flavor, green flavor, pea flavor, earthy flavor, hay-like flavor, grassy flavor, rancid flavor, leafy flavor, cardboard flavor, acrid flavor, pungent flavor, medicinal flavor, metallic flavor, and brothy flavor. In some cases, trained panelists can be able to discriminate between a protein composition provided herein and a different protein composition (e.g., a commercial protein product), or between food products containing them. In some embodiments, when assessed by a trained panel, the protein composition has a discriminability index of at least 1.0 (e.g., at least 1.5, 2.0, 2.5, or 3.0).

In some embodiments, other small molecules that are part of the source of the protein in the protein composition are also reduced in the protein composition as compared to the source of the protein in the protein composition as described herein. In some embodiments, a small molecule may have economic value outside of the context of a protein composition as described herein. In some embodiments, a protein composition can include less than 90% by mass (e.g., less than 80%, 70%, 60%, 50%, 40%, 30%, 20%, or 10%) by mass of one or more other small molecules. For example, when the source of the protein in the protein composition is soy, one or more isoflavones (e.g., genistein, daidzein, glycitein, or a combination thereof) can be depleted as compared to soy, or a defatted soy flour.

In some embodiments, a protein composition as described herein can include one or more added ingredients. In some cases, an added ingredient can be one or more of a preservative, an antioxidant, or a shelf life extender. Non-limiting examples of a preservative, antioxidant, or shelf life extender include 4-hexylresorcinol, acetic acid, ascorbic acid, ascorbyl palmitate, ascorbyl stearate, benzoic acid, butylated hydroxyanisole (a mixture of 2-tertiarybutyl-4-hydroxyanisole and 3-tertiarybutyl-4-hydroxyanisole), butylated hydroxytoluene (3,5-ditertiarybutyl-4-hydroxytoluene), calcium ascorbate, calcium propionate, calcium sorbate, Carnobacterium divergens M35, Carnobacterium maltaromaticum cb1, carnosum 4010, citric acid, a citric acid ester of a monoglyceride or diglyceride, dimethyl dicarbonate, erythorbic acid, ethyl lauroyl arginate, gum guaiacum, iso-ascorbic acid, L-cysteine, L-cysteine hydrochloride, lecithin, lecithin citrate, Leuconostoc, methyl paraben, methyl-p-hydroxybenzoate, monoglyceride citrate, monoisopropyl citrate, natamycin, nisin, potassium acetate, potassium benzoate, potassium bisulfite, potassium diacetate, potassium lactate, potassium metabisulfite, potassium nitrate, potassium nitrite, potassium sorbate, propionic acid, propyl gallate, propyl paraben, propyl-p-hydroxy benzoate, sodium acetate, sodium ascorbate, sodium benzoate, sodium bisulfite, sodium diacetate, sodium dithionite, sodium erythorbate, sodium iso-ascorbate, sodium lactate, sodium metabisulfite, sodium nitrate, sodium nitrite, sodium propionate, sodium salt of methyl-p-hydroxy benzoic acid, sodium salt of propyl-p-hydroxy benzoic acid, sodium sorbate, sodium sulfite, sorbic acid, sulfurous acid, tartaric acid, tertiary butyl hydroquinone, or a tocopherol.

A protein composition as described herein can be in any appropriate form. In some embodiments, a protein composition can be in the form of a solution, suspension, or emulsion. In some embodiments, a protein composition can be in the form of a solid or a powder. In some embodiments, a protein composition is in the form of an extrudate. An extrudate can, in some cases, be substantially in the form of granules. Granules can have an average largest dimension of about 3 mm to about 5 mm. In some embodiments, less than about 20% (w/w) of the granules can have a largest dimension less than 1 mm. In some embodiments, less than about 5% (w/w) of the granules can have a largest dimension over 1 cm. In some embodiments, an extrudate can have a bulk density of about 0.25 to about 0.4 g/cm³. In some embodiments, an extrudate can have a moisture content of about 5% to about 10%. In some embodiments, an extrudate can have a protein content of about 65% to about 100% by dry weight. In some embodiments, an extrudate can have a fat content of less than about 2%. In some embodiments, an extrudate can have a sugar content of less than about 1%. In some embodiments, an extrudate can have a hydration ratio of about 2.5 to about 3 after about 60 minutes of hydration at room temperature. In some embodiments, an extrudate can have a hydration time of less than about 30 minutes. In some embodiments, an extrudate can have a pH of about 5.0 to about 7.5 when hydrated. In some embodiments, an extrudate can have a bite strength of about 2000 g to about 4000 g at a hydration ratio of about 3.

Also provided herein are food products including any of the protein compositions as described herein, and/or protein compositions produced by any of the methods described herein. Food products as described herein can optionally further contain a fat (e.g., a non-animal fat) and one or more flavor precursor compounds. A food product can take any appropriate form, such as those described herein. In some embodiments, a food product can be a meat analog. In some embodiments, a food product can be beverage. In some embodiments, a food product can be a dairy replica (e.g., a milk replica).

As used herein, “a food product” means (1) articles used for food or drink for man or other animals, (2) chewing gum, and (3) articles used for components of any such article.

As used herein, “a plant-based food product” is a food product in which at least 50% (e.g., at least 60%, 70%, 80%, 90%, or more) by dry weight of the ingredients are from plants.

As used herein, “an algae-based food product” is a food product in which at least 50% (e.g., at least 60%, 70%, 80%, 90%, or more) by dry weight of the ingredients are from algae.

As used herein, “a fungus-based food product” is a food product in which at least 50% (e.g., at least 60%, 70%, 80%, 90%, or more) by dry weight of the ingredients are from fungus.

s used herein, “an invertebrate-based food product” is a food product in which at least 50% (e.g., at least 60%, 70%, 80%, 90%, or more) by dry weight of the ingredients are from invertebrates (e.g., insects and/or arachnids).

A protein composition as described herein or a protein composition produced by a method described herein can be included in a food product in any appropriate amount. For example, in some embodiments, a protein composition as described herein or a protein composition produced by a method described herein can be included in a food product in an amount of about 1% to about 99% (e.g., about 5% to about 80% or about 10% to about 30%) by dry weight of the food product.

In some embodiments, also provided herein are methods of preparing a food product, including combining a fat, one or more optional flavor precursor compounds, and a protein composition as described herein or a protein composition prepared by a method described herein.

In some embodiments, a food product as described herein can contain less than 10% (e.g., less than 5% or less than 1%) by weight animal products. In some embodiments, a food product can contain no animal products. In some embodiments, a food product can contain no animal meat. In some embodiments, a food product can contain no animal blood. In some embodiments, a food product can contain no animal products that contain heme.

A fat can be present in a food product in any appropriate amount. For example, a fat can be present in a lower amount in a low-fat meat analog (e.g., a chicken breast analog), or in a higher amount in a high-fat meat analog (e.g., a bacon analog). In some embodiments, a fat can be present in a low-fat meat analog in an amount of about 0.1% to about 5%. In some embodiments, a fat can be present in a fat tissue analog in an amount of about 85% to about 90%. In some embodiments, a ground meat analog can include about 10% to about 25% (e.g., about 10% to about 15%, about 10% to about 20%, about 15% to about 25%, or about 20% to about 25%) of a fat. In some embodiments, a milk replica can include about 0.01% to about 5% (e.g., about 0.01% to about 0.1%, about 0.1% to about 1%, or about 1% to about 5%) fat by weight of the milk replica.

Non-limiting examples of flavor precursor molecules include glucose, ribose, cysteine, a cysteine derivative, thiamine, alanine, methionine, lysine, a lysine derivative, glutamic acid, a glutamic acid derivative, IMP, GMP, lactic acid, maltodextrin, creatine, alanine, arginine, asparagine, aspartate, glutamic acid, glutamine, glycine, histidine, isoleucine, leucine, methionine, phenylalanine, proline, threonine, tryptophan, tyrosine, valine, linoleic acid, and mixtures thereof.

Also provided herein are methods of making food products. In some embodiments, the method can include combining a fat, one or more optional flavor precursor compounds, and any of the protein compositions as described herein (e.g., a low flavor protein isolate, or a low color protein composition, in the form of a protein isolate or a protein concentrate).

Also provided herein are methods of reducing perceived protein source flavor in a food product (e.g., a plant-based food product, an algae-based food product, a fungus-based food product, or an invertebrate-based food product). The method can include combining a fat, one or more optional flavor precursor compounds, and any of the protein compositions as described herein (e.g., a low flavor protein isolate, or a low color protein composition, in the form of a protein isolate or a protein concentrate), where at least 5% (e.g., at least 6%, 7%, 8%, 9%, 10%, 15%, 20%, 25%, or more) by weight of the protein content of the food product comprises the protein composition, as compared to a food product having a similar protein content but lacking the protein composition.

Any of the protein compositions described herein can be included in a variety of food products, including meat replicas, dairy replicas (e.g., milk replicas or cheese replicas), and beverages (e.g., protein supplement beverages, sports drink, protein shake, protein shot, energy drink, caffeinated beverage, coffee drink (e.g., milk coffee), milk, fermented milk, smoothie, carbonated beverage, alcoholic beverage, meal replacement beverages, or infant formula). In some cases, any of the protein compositions described herein can be sold to a consumer to be used in food products at the consumer’s discretion (e.g., to supplement a baked good with protein). Meat replicas can be formulated, for example, as ground meat (e.g., ground beef, pork, or chicken), sausages (e.g., breakfast sausages, bratwursts, or hot dogs), or as a cut of meat (e.g., a steak, a roast, a loin, a breast, a thigh, a leg, or a wing).

Exemplary food products are described in U.S. Pat. Nos. 10,039,306, 9,700,067, and 9,011,949; U.S. Pat. Application Publication Nos. US20150305361A1, US20170172169A1, US20150289541A1, and US20170188612A1, each of which is incorporated by reference in its entirety.

In some embodiments, a food product can be a protein supplement. For example, in some embodiments, a protein composition as disclosed herein can be part of a protein powder, which can be used in protein shakes, smoothies, baking, and the like.

In some embodiments, a food product can include a muscle replica. In some embodiments, a food product can include an adipose replica. In some embodiments, a food product can include a muscle replica and an adipose replica. In some embodiments, a food product that includes a muscle replica and an adipose replica can also be called a meat replica.

In some embodiments, a food product can be a dairy replica (e.g., a replica of milk, fermented milk, yogurt, cream, butter, cheese, custard, ice cream, gelato, or frozen yogurt). In some embodiments, a food product can be a cheese replica. In some embodiments, a food product can be a milk replica. In some embodiments, a milk replica comprising a protein composition as described herein can have one or more properties that are more like animal milk than other non-dairy milks including, for example, a whiter color, a better mouthfeel, a greater stability (e.g., a greater emulsion stability, a lack of curdling in hot or acidic liquids such as coffee), or a combination thereof. In some embodiments, a milk replica can have a protein content similar to or greater than that of cow’s milk. In some embodiments, a milk replica can have a protein content of about 20 to about 60 mg/mL (e.g., about 30 to about 55 mg/mL, about 25 to about 35 mg/mL), some or all of which can be a protein composition as described herein and/or a protein composition produced by a method described herein. For example, in some embodiments, the milk replica is stable (e.g., the emulsion does not break) when added to liquid with a temperature of about 70° C. to about 100° C. (e.g., about 80° C. to about 100° C., about 80° C. to about 98° C., about 70° C. to about 80° C., about 70° C. to about 95° C., about 70° C. to about 85° C., or about 80° C. to about 85° C.). In some embodiments, the milk replica is stable (e.g., the emulsion does not break) when added to liquid with a pH of about 4.0 to about 8.0 (e.g., about 4.0 to about 7.0, about 4.5 to about 6.5, about 4.5 to about 6.0). In some embodiments, a milk replica can be used to make a cheese replica.

In some embodiments, provided herein is a milk replica comprising an emulsion of a fat, water, and a protein composition as described herein or a protein composition produced by a method as described herein. In some embodiments, the fat is present in the milk replica in an amount of about 0.01% to about 5% (e.g., about 0.01% to about 0.1%, about 0.01% to about 0.5%, about 0.01% to about 1%, about 0.01% to about 2%, about 0.01% to about 3%, about 0.01 % to about 4%, about 0.1% to about 5%, about 0.5% to about 5%, about 1% to about 5%, about 2% to about 5%, about 3% to about 5%, or about 4% to about 5%) of the milk replica. In some embodiments, the fat is selected from the group consisting of corn oil, olive oil, soy oil, peanut oil, walnut oil, almond oil, sesame oil, cottonseed oil, rapeseed oil, canola oil, safflower oil, sunflower oil, flax seed oil, palm oil, palm kernel oil, coconut oil, babassu oil, shea butter, mango butter, cocoa butter, wheat germ oil, rice bran oil, and combinations thereof.

In some embodiments, a food product can be an egg replica. In some embodiments, a food product can be a whole egg replica (e.g., with a yolk replica partitioned from an albumen replica). In some embodiments, a food product can be an egg yolk replica. In some embodiments, a food product can be an albumen replica. In some embodiments, a food product can be a scrambled egg replica (e.g., a mixture of an egg yolk replica and an albumen replica).

A food product can include one or more proteins (e.g., a protein composition as described herein, a commercially available protein, a protein purified by any method known in the art, or a combination thereof). In some embodiments, a food product can include any of the protein compositions as described herein. In some embodiments, a food product can include any of the protein compositions as described herein in addition to a commercially available protein (e.g., soy protein concentrate, soy protein isolate, casein, whey, wheat gluten, pea vicilin, or pea legumin). In some embodiments, a food product can include any of the protein compositions as described herein, in addition to one or more proteins purified by any method known in the art.

One or more proteins (e.g., a protein composition as described herein, a commercially available protein, a protein purified by any method known in the art, or a combination thereof) can be present in an amount of about 0.1% to about 100% by weight (e.g., about 0.1% to about 1%, about 1% to about 5%, about 5% to about 10%, about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100% about 10% to about 30%, about 30% to about 50%, about 50% to about 70%, about 70% to about 90%, about 0.1% to about 20%, about 20% to about 40%, about 40% to about 60%, about 60% to about 80%, about 80% to about 100%, about 0.1% to about 33%, about 33% to about 66%, about 66% to about 100, about 0.1% to about 50%, or about 50% to about 100%) of a food product (e.g., a meat replica, a dairy replica, or a supplement).

Any of the food products described herein can include an iron complex (e.g., ferrous chlorophyllin (e.g., CAS No. 69138-22-3), iron pheophorbide (e.g., CAS No. 15664-29-6), an iron salt (e.g. iron sulfate (e.g., any of CAS Nos. 7720-78-7, 17375-41-6, 7782-63-0, or 10028-22-5) iron gluconate (e.g., any of CAS Nos. 299-29-6, 22830-45-1, or 699014-53-4), iron citrate (e.g., any of CAS Nos. 3522-50-7, 2338-05-8, or 207399-12-0), ferric EDTA (e.g., CAS No. 17099-81-9) or a heme (e.g., heme A (e.g., CAS No. 18535-39-2), heme B (e.g. CAS No. 14875-96-8), heme C (e.g., CAS No. 26598-29-8), heme O (e.g., CAS No. 137397-56-9), heme I, heme M, heme D, heme S)) or a heme-containing protein.

In some embodiments, a food product can include a heme-containing protein. In some embodiments, a food product can include a heme-containing protein in an amount of about 0.01% to about 5% (e.g., 0.01% to about 1%, about 0.01% to about 0.5%, about 0.01% to about 0.1%, about 0.01% to about 0.05%, about 0.05% to about 5%, about 0.1% to about 5%, about 0.5% to about 5%, about 1% to about 5%, about 0.05% to about 0.5%, or about 0.1% to about 0.5%) by weight of the food product. In some embodiments, the heme-containing protein is a globin. In some embodiments, the globin is selected from the group consisting of an androglobin, a cytoglobin, a globin E, a globin X, a globin Y, a hemoglobin, a myoglobin, a leghemoglobin, an erythrocruorin, a beta hemoglobin, an alpha hemoglobin, a protoglobin, a cyanoglobin, a cytoglobin, a histoglobin, a neuroglobins, a chlorocruorin, a truncated hemoglobin, a truncated 2/2 globin, and a hemoglobin 3. In some embodiments, the heme-containing protein is a non-animal heme-containing protein. In some embodiments, the heme-containing protein is a plant, fungal, algal, archaeal, or bacterial protein. In some embodiments, the heme-containing protein is not natively expressed in plant, fungal, algal, archaeal, or bacterial cells. In some embodiments, the heme-containing protein comprises an amino acid sequence having at least 50% sequence identity (e.g., at least 60%, 70%, 80%, 90%, or 95% sequence identity) to a polypeptide set forth in SEQ ID NOs. 1-27.

Heme-containing proteins that can be used in any of the food products described herein can be from mammals (e.g., farms animals such as cows, goats, sheep, pigs, ox, or rabbits), birds, plants, algae (e.g., C. reinhardtii), fungi (e.g., yeast or filamentous fungi), ciliates, or bacteria. For example, a heme-containing protein can be from a mammal such as a farm animal (e.g., a cow, goat, sheep, pig, ox, or rabbit) or a bird such as a turkey or chicken. Heme-containing proteins can be from a plant such as Nicotiana tabacum or Nicotiana sylvestris (tobacco); Zea mays (corn), Arabidopsis thaliana, a legume such as Glycine max (soybean), Cicer arietinum (garbanzo or chick pea), Pisum sativum (pea) varieties such as garden peas or sugar snap peas, Phaseolus vulgaris varieties of common beans such as green beans, black beans, navy beans, northern beans, or pinto beans, Vigna unguiculata varieties (cow peas), Vigna radiata (Mung beans), Lupinus albus (lupin), or Medicago sativa (alfalfa); Brassica napus (canola); Triticum sps. (wheat, including wheat berries, and spelt); Gossypium hirsutum (cotton); Oryza sativa (rice); Zizania sps. (wild rice); Helianthus annuus (sunflower); Beta vulgaris (sugarbeet); Pennisetum glaucum (pearl millet); Chenopodium sp. (quinoa); Sesamum sp. (sesame); Linum usitatissimum (flax); or Hordeum vulgare (barley). Heme-containing proteins can be isolated from fungi such as Saccharomyces cerevisiae, Pichia pastoris, Magnaporthe oryzae, Fusarium graminearum, Aspergillus oryzae, Trichoderma reesei, Myceliopthera thermophile, Kluyvera lactis, or Fusarium oxysporum. Heme-containing proteins can be isolated from bacteria such as Escherichia coli, Bacillus subtilis, Bacillus licheniformis, Bacillus megaterium, Synechocistis sp., Aquifex aeolicus, Methylacidiphilum infernorum, or thermophilic bacteria such as Thermophilus. The sequences and structure of numerous heme-containing proteins are known. See for example, Reedy, et al., Nucleic Acids Research, 2008, Vol. 36, Database issue D307-D313 and the Heme Protein Database available on the world wide web at hemeprotein.info/heme.php.

For example, a non-symbiotic hemoglobin can be from a plant selected from the group consisting of soybean, sprouted soybean, alfalfa, golden flax, black bean, black eyed pea, northern, garbanzo, moong bean, cowpeas, pinto beans, pod peas, quinoa, sesame, sunflower, wheat berries, spelt, barley, wild rice, or rice.

Any of the heme-containing proteins described herein that can be used for producing food products can have at least 70% (e.g., at least 75%, 80%, 85%, 90%, 95%, 97%, 98%, 99%, or 100%) sequence identity to the amino acid sequence of the corresponding wild-type heme-containing protein or fragments thereof that contain a heme-binding motif. For example, a heme-containing protein can have at least 70% sequence identity to an amino acid sequence, including a non-symbiotic hemoglobin such as that from Vigna radiata (SEQ ID NO:1), Hordeum vulgare (SEQ ID NO:5), Zea mays (SEQ ID NO:13), Oryza sativa subsp. japonica (rice) (SEQ ID NO:14), or Arabidopsis thaliana (SEQ ID NO:15), a Hell’s gate globin I such as that from Methylacidiphilum infernorum (SEQ ID NO:2), a flavohemoprotein such as that from Aquifex aeolicus (SEQ ID NO:3), a leghemoglobin such as that from Glycine max (SEQ ID NO:4), Pisum sativum (SEQ ID NO:16), or Vigna unguiculata (SEQ ID NO:17), a heme-dependent peroxidase such as from Magnaporthe oryzae, (SEQ ID NO:6) or Fusarium oxysporum (SEQ ID NO:7), a cytochrome c peroxidase from Fusarium graminearum (SEQ ID NO:8), a truncated hemoglobin from Chlamydomonas moewusii (SEQ ID NO:9), Tetrahymena pyriformis (SEQ ID NO:10, group I truncated), Paramecium caudatum (SEQ ID NO:11, group I truncated), a hemoglobin from Aspergillus niger (SEQ ID NO:12), or a mammalian myoglobin protein such as the Bos taurus (SEQ ID NO:18) myoglobin, Sus scrofa (SEQ ID NO:19) myoglobin, Equus caballus (SEQ ID NO:20) myoglobin, a heme-protein from Nicotiana benthamiana (SEQ ID NO:21), Bacillus subtilis (SEQ ID NO:22), Corynebacterium glutamicum (SEQ ID NO:23), Synechocystis PCC6803 (SEQ ID NO:24), Synechococcus sp. PCC 7335 (SEQ ID NO:25), Nostoc commune (SEQ ID NO:26), or Bacillus megaterium (SEQ ID NO: 27).

The percent identity between two amino acid sequences can be determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson’s web site (e.g., fr.com/blast/) or the U.S. government’s National Center for Biotechnology Information web site (ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Similar procedures can be following for nucleic acid sequences except that blastn is used.

Once aligned, the number of matches is determined by counting the number of positions where an identical amino acid residue is presented in both sequences. The percent identity is determined by dividing the number of matches by the length of the full-length polypeptide amino acid sequence followed by multiplying the resulting value by 100. It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

In some embodiments, heme-containing proteins can be extracted from a production organism (e.g., extracted from animal tissue, or plant, fungal, algal, or bacterial biomass, or from the culture supernatant for secreted proteins) or from a combination of production organisms (e.g., multiple plant species). Leghemoglobin is readily available as an unused by-product of commodity legume crops (e.g., soybean, alfalfa, or pea). The amount of leghemoglobin in the roots of these crops in the United States exceeds the myoglobin content of all the red meat consumed in the United States.

In some embodiments, extracts of heme-containing proteins include one or more non -heme-containing proteins from the source material (e.g., other animal, plant, fungal, algal, or bacterial proteins) or from a combination of source materials (e.g., different animal, plant, fungi, algae, or bacteria).

In some embodiments, heme-containing proteins can be provided in a food product in a form that is not part of a protein composition as described herein. In some embodiments, heme-containing proteins can be purified by any method known in the art.

Also provided herein is a method of evaluating a protein composition for effect on flavor in a food product, the method including determining that a level of one or more volatile compounds in a set of volatile compounds of a first protein composition from a protein source is higher than the level of the one or more volatile compounds of a second protein composition from the protein source; and determining that the second protein composition is superior to the first protein composition for use in a food product. In some embodiments, the second protein composition is a protein composition as described herein or a protein composition produced by a method described herein. In some embodiments, the first protein composition is not a protein composition described herein or a protein composition produced by a method described herein.

Also provided herein is a method of evaluating a protein composition for effect on flavor in a food product, the method including determining that a level of one or more volatile compounds in a set of volatile compounds of a source protein composition from a protein source is higher than the level of the one or more volatile compounds of a protein composition from the protein source; and determining that the protein composition is superior to the source protein composition for use in a food product. In some embodiments, the protein composition is a protein composition as described herein, or a protein composition produced by a method described herein.

In some embodiments, the set of volatile compounds comprises a volatile compound from any one of volatile sets 1-10. In some embodiments, the set of volatile compounds is any one of volatile sets 1-10. In some embodiments, wherein the set of volatile compounds is selected from the group consisting of volatile set 1, volatile set 2, volatile set 3, volatile set 4, volatile set 5, volatile set 6, volatile set 7, volatile set 8, volatile set 9, volatile set 10, and combinations thereof. In some embodiments, the protein source is a plant, a fungus, algae, bacteria, protozoa, an invertebrate, or a combination thereof. In some embodiments, the protein source is soy. In some embodiments, set of volatile compounds comprise at least one compound selected from the group consisting of hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal. In some embodiments, set of volatile compounds is hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal. In some embodiments, the food product is a meat replica. In some embodiments, the food product is plant-based. In some embodiments, n the food product contains less than 10% by weight animal products. In some embodiments, the food product contains less than 5% by weight animal products. In some embodiments, the food product contains less than 1% by weight animal products. In some embodiments, the food product contains no animal products.

Also provided here in is a method of reducing flavor in a protein composition, the method including (a) determining a level of one or more volatile compounds in a set of volatile compounds of a first protein composition from a protein source; (b) preparing a second protein composition from the protein source, wherein preparing the second protein composition comprises reducing the amount of one or more components of the protein source that are included in the second protein composition; and (c) determining that a level of one or more volatile compounds in a set of volatile compounds from the second protein composition is lower than the level of the one or more volatile compounds in a set of volatile compounds in the first protein composition.

Also provided herein is a method of determining a cause of flavor in a protein composition, the method including (a) determining a level of one or more volatile compounds in a set of volatile compounds of a first protein composition from a protein source; (b) providing a second protein composition from the protein source, wherein the second protein composition comprises a decreased amount of one or more components of the protein source; (c) determining that a level of one or more volatile compounds in a set of volatile compounds from the second protein composition is lower than the level the of one or more volatile compounds in a set of volatile compounds in the first protein composition; and (d) identifying the one or more components of the protein course to be a cause of flavor in the protein composition.

In some embodiments, the second protein composition can be a protein composition as described herein, or a protein composition produced by a method described herein. In some embodiments, the set of volatile compounds comprises a volatile compound from any one of volatile sets 1-10. In some embodiments, the set of volatile compounds is any one of volatile sets 1-10. In some embodiments, the set of volatile compounds is selected from the group consisting of volatile set 1, volatile set 2, volatile set 3, volatile set 4, volatile set 5, volatile set 6, volatile set 7, volatile set 8, volatile set 9, volatile set 10, and combinations thereof. In some embodiments, the protein source is a plant, a fungus, algae, bacteria, protozoa, an invertebrate, or a combination thereof. In some embodiments, the protein source is soy. In some embodiments, the set of volatile compounds comprise at least one compound selected from the group consisting of hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal. In some embodiments, the set of volatile compounds is hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal. In some embodiments, the component of the protein source that is decreased comprises lipids. In some embodiments, the component of the protein source that is decreased comprises a fatty acid, a wax, a sterol, a monoglyceride, a diglyceride, a triglyceride, a sphingolipid, phospholipid, or a combination thereof. In some embodiments, the component of the protein source that is decreased comprises phospholipids. In some embodiments, the decreased amount of one or more components of the protein source in the second protein composition is at least a 10% decrease (e.g., at least a 30%, 50%, 70%, or 90% decrease) compared to the first protein composition.

### Exemplary Embodiments

Embodiment 1 is a protein composition comprising:
at least 50% by dry weight of a plurality of plant proteins, fungal proteins, algal proteins, bacterial proteins, protozoan proteins, invertebrate proteins, or a combination thereof,
wherein the protein composition is a low color protein composition.

Embodiment 2 is a protein composition comprising:
at least 50% by dry weight of a plurality of plant proteins, fungal proteins, algal proteins, bacterial proteins, protozoan proteins, invertebrate proteins, or a combination thereof;
less than 1.0% by dry weight of lipids.

Embodiment 3 is a protein composition produced by a method comprising:
(a) adding an aqueous solution to a source protein composition to form a solution of solubilized protein;
(b) optionally removing solids from the solution of solubilized protein;
(c) optionally heating the solution of solubilized protein;
(d) optionally adjusting the pH of the solution of solubilized protein to about 4.0 to about 9.0;
(e) optionally cooling the solution of solubilized protein to about 0° C. to about 10° C.;
(f) adding an organic solvent to the solution of solubilized protein to form a solid phase and a liquid phase;
(g) separating the solid phase from the liquid phase to form the protein composition;
(h) optionally washing the protein composition with a wash solvent; and
(i) optionally treating the protein composition,
wherein the protein composition comprises at least at least 50% by dry weight of a plurality of plant proteins, fungal proteins, algal proteins, bacterial proteins, protozoan proteins, invertebrate proteins.

Embodiment 4 is the protein composition of any one of embodiments 2-3, wherein the protein composition is a low color protein composition.

Embodiment 5 is the protein composition of any one of embodiments 1-4, wherein the protein composition comprises at least about 90% by dry weight of the plurality of plant proteins, fungal proteins, algal proteins, bacterial proteins, protozoan proteins, invertebrate proteins, or the combination thereof.

Embodiment 6 is the protein composition of any one of embodiments 1-4, wherein the protein composition comprises at least about 91% by dry weight of the plurality of plant proteins, fungal proteins, algal proteins, bacterial proteins, protozoan proteins, invertebrate proteins, or the combination thereof.

Embodiment 7 is the protein composition of any one of embodiments 1-4, wherein the protein composition comprises at least about 93% by dry weight of the plurality of plant proteins, fungal proteins, algal proteins, bacterial proteins, protozoan proteins, invertebrate proteins, or the combination thereof.

Embodiment 8 is the protein composition of any one of embodiments 5-7, wherein the protein composition is a protein isolate.

Embodiment 9 is the protein composition of embodiment 8, wherein the protein composition comprises less than 8% by dry weight of insoluble carbohydrates.

Embodiment 10 is the protein composition of any one of embodiments 8-9, wherein the protein composition is a low flavor protein composition.

Embodiment 11 is the protein composition of any one of embodiments 8-10, wherein the protein composition has an isoflavone content of less than about 150 ppm.

Embodiment 12 is the protein composition of any one of embodiments 8-11, wherein the protein composition has an isoflavone content of less than about 125 ppm.

Embodiment 13 is the protein composition of any one of embodiments 8-12, wherein the protein composition has an isoflavone content of less than about 100 ppm.

Embodiment 14 is the protein composition of any one of embodiments 8-13, wherein the protein composition has an isoflavone content of less than about 75 ppm.

Embodiment 15 is the protein composition of any one of embodiments 8-14, wherein the protein composition has a saponin content of less than about 75 ppm.

Embodiment 16 is the protein composition of any one of embodiments 8-15, wherein the protein composition has a saponin content of less than about 50 ppm.

Embodiment 17 is the protein composition of any one of embodiments 8-16, wherein the protein composition has a saponin content of less than about 25 ppm.

Embodiment 18 is the protein composition of any one of embodiments 8-17, wherein the protein composition has a phospholipid content of less than about 500 ppm.

Embodiment 19 is the protein composition of any one of embodiments 8-18, wherein the protein composition has a phospholipid content of less than about 250 ppm.

Embodiment 20 is the protein composition of any one of embodiments 8-19, wherein the protein composition has a phospholipid content of less than about 100 ppm.

Embodiment 21 is the protein composition of any one of embodiments 8-20, wherein the protein composition has a phospholipid content of less than about 50 ppm.

Embodiment 22 is the protein composition of any one of embodiments 8-21, wherein the protein composition has a phospholipid content of less than about 25 ppm.

Embodiment 23 is the protein composition of any one of embodiments 8-22, wherein the protein composition has a phospholipid content of less than about 10 ppm.

Embodiment 24 is the protein composition of any one of embodiments 8-23, wherein the protein composition has a phospholipid content of less than about 5 ppm.

Embodiment 25 is the protein composition of any one of embodiments 8-24, wherein the protein composition has a phospholipid content of less than about 2 ppm.

Embodiment 26 is the protein composition of any one of embodiments 8-25, wherein the protein composition has a phospholipid content of less than about 1 ppm.

Embodiment 27 is the protein composition of any one of embodiments 1-5, wherein the protein composition comprises about 60% to about 80% by dry weight of the plurality of plant proteins, fungal proteins, algal proteins, bacterial proteins, protozoan proteins, invertebrate proteins, or the combination thereof.

Embodiment 28 is the protein composition of embodiment 27, wherein the protein composition comprises about 65% to about 75% by dry weight of the plurality of plant proteins, fungal proteins, algal proteins, bacterial proteins, protozoan proteins, invertebrate proteins, or the combination thereof.

Embodiment 29 is the protein composition of embodiment 27 or embodiment 28, wherein the protein composition is a protein concentrate.

Embodiment 30 is the protein composition of embodiment 29, wherein the protein composition comprises at least 9% by dry weight of insoluble carbohydrates.

Embodiment 31 is the protein composition of any one of embodiments 1-30, wherein the protein composition comprises less than 0.8% by dry weight of lipids.

Embodiment 32 is the protein composition of any one of embodiments 1-31, wherein the protein composition comprises less than 0.6% by dry weight of lipids.

Embodiment 33 is the protein composition of any one of embodiments 1-32, wherein the protein composition comprises less than 0.4% by dry weight of lipids.

Embodiment 34 is the protein composition of any one of embodiments 1-33, wherein the protein composition has a luminance of at least 86 on a scale from 0 (black control value) to 100 (white control value).

Embodiment 35 is the protein composition of any one of embodiments 1-34, wherein the protein composition has a luminance of at least 88 on a scale from 0 (black control value) to 100 (white control value).

Embodiment 36 is the protein composition of any one of embodiments 1-35, wherein the protein composition has a luminance of at least 90 on a scale from 0 (black control value) to 100 (white control value).

Embodiment 37 is the protein composition of any one of embodiments 1-36, wherein the protein composition has a chroma value of less than 14.

Embodiment 38 is the protein composition of any one of embodiments 1-37, wherein the protein composition has a chroma value of less than 12.

Embodiment 39 is the protein composition of any one of embodiments 1-38, wherein the protein composition has a chroma value of less than 10.

Embodiment 40 is the protein composition of any one of embodiments 1-39, wherein the protein composition has a chroma value of less than 8.

Embodiment 41 is the protein composition of any one of embodiments 1-40, wherein the protein composition has a chroma value of less than 6.

Embodiment 42 is the protein composition of any one of embodiments 1-41, wherein the plurality of plant proteins, fungal proteins, algal proteins, bacterial proteins, protozoan proteins, invertebrate proteins, or the combination thereof comprises at least 90% plant proteins.

Embodiment 43 is the protein composition of any one of embodiments 1-41, wherein the plurality of plant proteins, fungal proteins, algal proteins, bacterial proteins, protozoan proteins, invertebrate proteins, or the combination thereof comprises at least 90% legume proteins.

Embodiment 44 is the protein composition of any one of embodiments 1-41, wherein the plurality of plant proteins, fungal proteins, algal proteins, bacterial proteins, protozoan proteins, invertebrate proteins, or the combination thereof comprises at least 90% pulse proteins.

Embodiment 45 is the protein composition of any one of embodiments 1-41, wherein the plurality of plant proteins, fungal proteins, algal proteins, bacterial proteins, protozoan proteins, invertebrate proteins, or the combination thereof comprises at least 90% soy proteins.

Embodiment 46 is the protein composition of any one of embodiments 1-41, wherein the plurality of plant proteins, fungal proteins, algal proteins, bacterial proteins, protozoan proteins, invertebrate proteins, or the combination thereof comprises at least 90% fungal proteins.

Embodiment 47 is the protein composition of any one of embodiments 1-41, wherein the plurality of plant proteins, fungal proteins, algal proteins, bacterial proteins, protozoan proteins, invertebrate proteins, or the combination thereof comprises at least 90% algal proteins.

Embodiment 48 is the protein composition of any one of claims 1-47, wherein wherein the plurality of plant proteins, fungal proteins, algal proteins, bacterial proteins, protozoan proteins, invertebrate proteins, or the combination thereof are substantially denatured, aggregated, or both.

Embodiment 49 is the protein composition of any one of embodiments 1-48, wherein when cooked in a solution comprising a reducing sugar, a sulfur-containing amino acid, and a heme-containing protein, a 1% (w/v) of the protein composition produces one or more volatile compounds associated with the aroma and/or taste of meat.

Embodiment 50 is the protein composition of embodiment 49, wherein at least one of the one or more volatile compounds associated with the aroma and/or taste of meat is produced in a smaller amount when the reducing sugar, the sulfur-containing amino acid, and the heme-containing protein are cooked in the absence of the protein composition.

Embodiment 51 is the protein composition of embodiment 49, wherein at least one of the one or more volatile compounds associated with the aroma and/or taste of meat is not produced when the reducing sugar, the sulfur-containing amino acid, and the heme-containing protein are cooked in the absence of the protein composition.

Embodiment 52 is the protein composition of any one of embodiments 49-51, wherein the one or more volatile compounds associated with the aroma and/or taste of meat comprise at least one compound selected from the group consisting of 2,3-butanedione, 2,3-pentanedione, thiazole, 2-acetylthiazole, benzaldehyde, 3-methyl-butanal, 2-methyl-butanal, thiophene, pyrazine, and combinations thereof.

Embodiment 53 is the protein composition of any one of embodiments 1-52, wherein when assessed by a trained descriptive panel using the Spectrum method, the protein composition is described as having low intensity of one or more of: oxidized/rancid flavor, cardboard flavor, astringent flavor, bitter flavor, vegetable complex flavor, and sweet fermented flavor.

Embodiment 54 is the protein composition of any one of embodiments 1-52, wherein when assessed by a trained descriptive panel using the Spectrum method, the protein composition is described as having low intensity of one or more of: beany flavor, fatty flavor, green flavor, pea flavor, earthy flavor, hay-like flavor, grassy flavor, rancid flavor, leafy flavor, cardboard flavor, acrid flavor, pungent flavor, medicinal flavor, metallic flavor, and brothy flavor.

Embodiment 55 is the protein composition of any one of embodiments 1-54, wherein when assessed by a trained panel, the protein composition has a discriminability index of at least 1.0.

Embodiment 56 is the protein composition of any one of embodiments 1-55, wherein when assessed by a trained panel, the protein composition has a discriminability index of at least 1.5.

Embodiment 57 is the protein composition of any one of embodiments 1-56, wherein when assessed by a trained panel, the protein composition has a discriminability index of at least 2.0.

Embodiment 58 is the protein composition of any one of embodiments 1-57, wherein when assessed by a trained panel, the protein composition has a discriminability index of at least 2.5.

Embodiment 59 is the protein composition of any one of embodiments 1-58, wherein when assessed by a trained panel, the protein composition has a discriminability index of at least 3.0.

Embodiment 60 is the protein composition of any one of embodiments 1-59, wherein the protein composition comprises less than about 0.5% by dry weight phospholipids.

Embodiment 61 is the protein composition of any one of embodiments 1-60, wherein the plurality of plant proteins, fungal proteins, algal proteins, bacterial proteins, protozoan proteins, invertebrate proteins, or a combination thereof is at least 90% by dry weight soy proteins.

Embodiment 62 is the protein composition of any one of embodiments 1-61, further comprising at least one of a preservative, an antioxidant, or a shelf life extender.

Embodiment 63 is the protein composition of embodiment 62, wherein the preservative, antioxidant, or shelf life extender comprises at least one of 4-hexylresorcinol, acetic acid, ascorbic acid, ascorbyl palmitate, ascorbyl stearate, benzoic acid, butylated hydroxyanisole (a mixture of 2-tertiarybutyl-4-hydroxyanisole and 3-tertiarybutyl-4-hydroxyanisole), butylated hydroxytoluene (3,5-ditertiarybutyl-4-hydroxytoluene), calcium ascorbate, calcium propionate, calcium sorbate, Carnobacterium divergens M35, Carnobacterium maltaromaticum cb1, carnosum 4010, citric acid, a citric acid ester of a monoglyceride or diglyceride, dimethyl dicarbonate, erythorbic acid, ethyl lauroyl arginate, gum guaiacum, iso-ascorbic acid, L-cysteine, L-cysteine hydrochloride, lecithin, lecithin citrate, Leuconostoc, methyl paraben, methyl-p-hydroxybenzoate, monoglyceride citrate, monoisopropyl citrate, natamycin, nisin, potassium acetate, potassium benzoate, potassium bisulfite, potassium diacetate, potassium lactate, potassium metabisulfite, potassium nitrate, potassium nitrite, potassium sorbate, propionic acid, propyl gallate, propyl paraben, propyl-p-hydroxy benzoate, sodium acetate, sodium ascorbate, sodium benzoate, sodium bisulfite, sodium diacetate, sodium dithionite, sodium erythorbate, sodium iso-ascorbate, sodium lactate, sodium metabisulfite, sodium nitrate, sodium nitrite, sodium propionate, sodium salt of methyl-p-hydroxy benzoic acid, sodium salt of propyl-p-hydroxy benzoic acid, sodium sorbate, sodium sulfite, sorbic acid, sulfurous acid, tartaric acid, tertiary butyl hydroquinone, or a tocopherol.

Embodiment 64 is the protein composition of any one of embodiments 1-63, wherein the protein composition is in the form of a solution, suspension, or emulsion.

Embodiment 65 is the protein composition of any one of embodiments 1-63, wherein the protein composition is in the form of a solid or a powder.

Embodiment 66 is the protein composition of embodiment 65, wherein the protein composition has an average particle size of about 5 µm to about 40 µm in the largest dimension.

Embodiment 67 is the protein composition of embodiment 65, wherein the protein composition has an average particle size of about 10 µm to about 40 µm in the largest dimension.

Embodiment 68 is the protein composition of embodiment 65, wherein the protein composition has an average particle size of about 10 µm to about 30 µm in the largest dimension.

Embodiment 69 is the protein composition of embodiment 65, wherein the protein composition has an average particle size of about 10 µm to about 20 µm in the largest dimension.

Embodiment 70 is the protein composition of any one of embodiments 1-69, wherein the protein composition is in the form of an extrudate.

Embodiment 71 is the protein composition of embodiment 70, wherein the extrudate is substantially in the form of granules.

Embodiment 72 is the protein composition of embodiment 71, wherein the granules have an average largest dimension of about 3 mm to about 5 mm.

Embodiment 73 is the protein composition of embodiment 71 or embodiment 72, wherein less than about 20% (w/w) of the granules have a largest dimension less than 1 mm.

Embodiment 74 is the protein composition of any one of embodiments 71-73, wherein less than about 5% (w/w) of the granules have a largest dimension over 1 cm.

Embodiment 75 is the protein composition of any one of embodiments 70-74, wherein the extrudate has a bulk density of about 0.25 to about 0.4 g/cm³.

Embodiment 76 is the protein composition of any one of embodiments 70-75, wherein the extrudate has a moisture content of about 5% to about 10%.

Embodiment 77 is the protein composition of any one of embodiments 70-76, wherein the extrudate has a protein content of about 65% to about 100% by dry weight.

Embodiment 78 is the protein composition of any one of embodiments 70-77, wherein the extrudate has a fat content of less than about 1.0%.

Embodiment 79 is the protein composition of any one of embodiments 70-78, wherein the extrudate has a sugar content of less than about 1%.

Embodiment 80 is the protein composition of any one of embodiments 70-79, wherein the extrudate has a hydration ratio of about 2.5 to about 3 after about 60 minutes of hydration at room temperature.

Embodiment 81 is the protein composition of any one of embodiments 70-80, wherein the extrudate has a hydration time of less than about 30 minutes.

Embodiment 82 is the protein composition of any one of embodiments 70-81, wherein the extrudate has a pH of about 5.0 to about 7.5 when hydrated.

Embodiment 83 is the protein composition of any one of embodiments 70-82, wherein the extrudate has a bite strength of about 2000 g to about 4000 g at a hydration ratio of about 3.

Embodiment 84 is the protein composition of embodiment 3 or any one of embodiments 4-83 as dependent on embodiment 3, wherein step (a) is performed at a pH of about 7.0 to about 10.0.

Embodiment 85 is the protein composition of embodiment 3 or any one of embodiments 4-84 as dependent on embodiment 3, wherein step (a) is performed at a pH of about 6.0 to about 9.0.

Embodiment 86 is the protein composition of embodiment 3 or any one of embodiments 4-85 as dependent on embodiment 3, wherein step (a) is performed at a pH of about 7.5 to about 8.5.

Embodiment 87 is the protein composition of embodiment 3 or any one of embodiments 4-86 as dependent on embodiment 3, wherein step (b) comprises centrifugation, filtration, or a combination thereof.

Embodiment 88 is the protein composition of embodiment 3 or any one of embodiments 4-87 as dependent on embodiment 3, wherein step (d) comprises adjusting the pH of the solution of solubilized protein to about 4.0 to about 6.0.

Embodiment 89 is the protein composition of embodiment 3 or any one of embodiments 4-88 as dependent on embodiment 3, wherein step (d) comprises adjusting the pH of the solution of solubilized protein to about 6.0 to about 7.0.

Embodiment 90 is the protein composition of embodiment 3 or any one of embodiments 4-89 as dependent on embodiment 3, wherein step (f) comprises adding the organic solvent to a final concentration of about 5% to about 70% (v/v).

Embodiment 91 is the protein composition of embodiment 3 or any one of embodiments 4-90 as dependent on embodiment 3, wherein step (f) comprises adding the organic solvent to a final concentration of about 10% to about 50% (v/v).

Embodiment 92 is the protein composition of embodiment 3 or any one of embodiments 4-90 as dependent on embodiment 3, wherein step (f) comprises adding the organic solvent to a final concentration of about 40% to about 70% (v/v).

Embodiment 93 is the protein composition embodiment 3 or any one of embodiments 4-92 as dependent on embodiment 3, wherein at the beginning of step (f), the organic solvent has a temperature of about -20° C. to about 10° C.

Embodiment 94 is the protein composition of embodiment 3 or any one of embodiments 4-93 as dependent on embodiment 3, wherein at the beginning of step (f), the organic solvent has a temperature of about -20° C. to about 0° C.

Embodiment 95 is the protein composition of embodiment 3 or any one of embodiments 4-93 as dependent on embodiment 3, wherein at the beginning of step (f), the organic solvent has a temperature of about 0° C. to about 4° C.

Embodiment 96 is the protein composition of embodiment 3 or any one of embodiments 4-92 as dependent on embodiment 3, wherein at the beginning of step (f), the organic solvent has a temperature of about 10° C. to about 25° C.

Embodiment 97 is the protein composition of embodiment 3 or any one of embodiments 4-96 as dependent on embodiment 3, wherein step (e) comprises cooling the solution of solubilized protein to a temperature of about 0° C. to about 4° C.

Embodiment 98 is the protein composition embodiment 3 or any one of embodiments 4-96 as dependent on embodiment 3, wherein at the beginning of step (f), the solution of solubilized protein has a temperature of about 10° C. to about 25° C.

Embodiment 99 is the protein composition of embodiment 3 or any one of embodiments 4-98 as dependent on embodiment 3, wherein step (c) comprises heating the solution of solubilized protein for a period of about 10 seconds to about 30 minutes.

Embodiment 100 is the protein composition of embodiment 3 or any one of embodiments 4-99 as dependent on embodiment 3, wherein step (c) comprises heating the solution of solubilized protein for a period of about 1 minute to about 20 minutes.

Embodiment 101 is the protein composition of embodiment 3 or any one of embodiments 4-100 as dependent on embodiment 3, wherein step (c) comprises heating the solution of solubilized protein at a temperature of about 70° C. to about 100° C.

Embodiment 102 is the protein composition of embodiment 3 or any one of embodiments 4-101 as dependent on embodiment 3, wherein step (c) comprises heating the solution of solubilized protein at a temperature of about 85° C. to about 95° C.

Embodiment 103 is the protein composition of embodiment 3 or any one of embodiments 4-102 as dependent on embodiment 3, wherein step (g) comprises centrifugation, filtration, or a combination thereof.

Embodiment 104 is the protein composition of embodiment 3 or any one of embodiments 4-103 as dependent on embodiment 3, wherein the organic solvent is selected from the group consisting of ethanol, methanol, propanol, isopropyl alcohol, and acetone.

Embodiment 105 is the protein composition of embodiment 3 or any one of embodiments 4-104 as dependent on embodiment 3, wherein the organic solvent is ethanol.

Embodiment 106 is the protein composition of embodiment 3 or any one of embodiments 4-105 as dependent on embodiment 3, wherein the wash solvent is an organic wash solvent.

Embodiment 107 is the protein composition of embodiment 106, wherein the organic wash solvent is the same as the organic solvent in step (f).

Embodiment 108 is the protein composition of embodiment 106, wherein the organic wash solvent is selected from the group consisting of ethanol, methanol, propanol, isopropyl alcohol, and acetone.

Embodiment 109 is the protein composition of embodiment 106, wherein the organic wash solvent is ethanol.

Embodiment 110 is the protein composition of embodiment 3 or any one of embodiments 4-105 as dependent on embodiment 3, wherein the wash solvent is an aqueous solution.

Embodiment 111 is the protein composition of embodiment 3 or any one of embodiments 4-105, wherein the wash solvent is a mixture of an aqueous solution and an organic wash solvent.

Embodiment 112 is the protein composition of embodiment 111, wherein the wash solvent comprises about 1% to about 30% (v/v) of the organic wash solvent.

Embodiment 113 is the protein composition of embodiment 111, wherein the wash solvent comprises about 30% to about 80% (v/v) of the organic wash solvent.

Embodiment 114 is the protein composition of embodiment 111, wherein the wash solvent comprises about 80% to about 99% (v/v) of the organic wash solvent.

Embodiment 115 is the protein composition of any one of embodiments 111-114, wherein the organic wash solvent is ethanol.

Embodiment 116 is the protein composition of any one of embodiments 111-114, wherein the organic wash solvent in step (h) is the same as the organic solvent in step (f).

Embodiment 117 is the protein composition of embodiment 3 or any one of embodiments 4-116 as dependent on embodiment 3, wherein the treating comprises resolubilizing the protein composition to a concentration of about 1.5 to about 50 mg/mL.

Embodiment 118 is the protein composition of embodiment 3 or any one of embodiments 4-117 as dependent on embodiment 3, wherein the treating comprises resolubilizing the protein composition to a concentration of about 2 to about 4 mg/mL.

Embodiment 119 is the protein composition of embodiment 3 or any one of embodiments 4-117 as dependent on embodiment 3, wherein the treating comprises resolubilizing the protein composition to a concentration of about 20 to about 40 mg/mL.

Embodiment 120 is the protein composition of embodiment 3 or any one of embodiments 4-119 as dependent on embodiment 3, wherein the treating comprises resolubilizing at least a portion of the protein composition at a pH of at least 8.0.

mbodiment 121 is the protein composition of embodiment 120, wherein the treating comprises resolubilizing at least a portion of the protein composition at a pH of at least 9.0.

Embodiment 122 is the protein composition of embodiment 121, wherein the treating comprises resolubilizing at least a portion of the protein composition at a pH of at least 10.0.

Embodiment 123 is the protein composition of any one of embodiments 120-122, further comprising neutralizing or acidifying the protein composition.

Embodiment 124 is the protein composition of embodiment 3 or any one of embodiments 4-123 as dependent on embodiment 3, wherein the treating comprises resolubilizing at least a portion of the protein composition using an enzyme.

Embodiment 125 is the protein composition of embodiment 121, wherein the enzyme is a protein deamidase.

Embodiment 126 is the protein composition of embodiment 121, wherein the enzyme is a protein glutaminase.

Embodiment 127 is the protein composition of embodiment 121, wherein the enzyme is a protein asparaginase.

Embodiment 128 is the protein composition of embodiment 3 or any one of embodiments 4-127 as dependent on embodiment 3 comprising steps (a), (b), (f), and (g).

Embodiment 129 is the protein composition of embodiment 3 or any one of embodiments 4-128 as dependent on embodiment 3 comprising steps (a), (b), (c), (f), and (g).

Embodiment 130 is the protein composition of embodiment 129, wherein step (c) follows step (b).

Embodiment 131 is the protein composition of embodiment 129, wherein step (b) follows step (c).

Embodiment 132 is the protein composition of embodiment 3 or any one of embodiments 4-131 as dependent on embodiment 3 comprising steps (a), (b), (d), (f), and (g).

Embodiment 133 is the protein composition of embodiment 132, wherein step (d) follows step (b).

Embodiment 134 is the protein composition of embodiment 3 or any one of embodiments 4-133 as dependent on embodiment 3 comprising steps (a), (b), (e), (f), and (g).

Embodiment 135 is the protein composition of embodiment 134, wherein step (e) follows step (b).

Embodiment 136 is the protein composition of embodiment 134, wherein step (b) follows step (e).

Embodiment 137 is the protein composition of embodiment 3 or any one of embodiments 4-136 as dependent on embodiment 3 comprising steps (a), (b), (c), (d), (f), and (g).

Embodiment 138 is the protein composition of embodiment 137, wherein steps (b), (c), and (d) are performed in the order of (b), (c), (d).

Embodiment 139 is the protein composition of embodiment 137, wherein steps (b), (c), and (d) are performed in the order of (c), (b), (d).

Embodiment 140 is the protein composition of embodiment 137, wherein steps (b), (c), and (d) are performed in the order of (b), (d), (c).

Embodiment 141 is the protein composition of embodiment 3 or any one of embodiments 4-140 as dependent on embodiment 3 comprising steps (a), (b), (c), (e), (f), and (g).

Embodiment 142 is the protein composition of embodiment 141, wherein steps (b), (c), and (e) are performed in the order of (b), (c), (e).

Embodiment 143 is the protein composition of embodiment 141, wherein steps (b), (c), and (e) are performed in the order of (c), (b), (e).

Embodiment 144 is the protein composition of embodiment 141, wherein steps (b), (c), and (e) are performed in the order of (b), (e), (c).

Embodiment 145 is the protein composition of embodiment 3 or any one of embodiments 4-144 as dependent on embodiment 3 comprising steps (a), (b), (c), (d), (e), (f), and (g).

Embodiment 146 is the protein composition of embodiment 146, wherein steps (b), (c), (d), and (e) are performed in the order of (b), (c), (d), (e).

Embodiment 147 is the protein composition of embodiment 146, wherein steps (b), (c), (d), and (e) are performed in the order of (c), (b), (d), (e).

Embodiment 148 is the protein composition of embodiment 146, wherein steps (b), (c), (d), and (e) are performed in the order of (b), (d), (e), (c).

Embodiment 149 is the protein composition of embodiment 146, wherein steps (b), (c), (d), and (e) are performed in the order of (b), (d), (c), (e).

Embodiment 150 is the protein composition of embodiment 3 or any one of embodiments 4-149 as dependent on embodiment 3 comprising steps (a), (c), (f), and (g).

Embodiment 151 is the protein composition of embodiment 3 or any one of embodiments 4-149 as dependent on embodiment 3 comprising steps (a), (c), (d), (f), and (g).

Embodiment 152 is the protein composition of embodiment 151, wherein step (c) is performed before step (d).

Embodiment 153 is the protein composition of embodiment 151, wherein step (d) is performed before step (c).

Embodiment 154 is the protein composition of embodiment 3 or any one of embodiments 4-153 as dependent on embodiment 3 comprising steps (a), (c), (d), (e), (f), and (g).

Embodiment 155 is the protein composition of embodiment 154, wherein steps (c), (d), and (e) are performed in the order (c), (d), (e).

Embodiment 156 is the protein composition of embodiment 154, wherein steps (c), (d), and (e) are performed in the order (d), (e), (c).

Embodiment 157 is the protein composition of embodiment 154, wherein steps (c), (d), and (e) are performed in the order (d), (c), (e).

Embodiment 158 is the protein composition of embodiment 3 or any one of embodiments 4-157 as dependent on embodiment 3 comprising steps (a), (d), (f), and (g).

Embodiment 159 is the protein composition of embodiment 3 or any one of embodiments 4-158 as dependent on embodiment 3 comprising steps (a), (d), (e), (f), and (g).

Embodiment 160 is the protein composition of embodiment 3, wherein step (d) is performed before step (e).

Embodiment 161 is the protein composition of embodiment 3 or any one of embodiments 4-160 as dependent on embodiment 3, comprising steps (a), (e), (f), and (g).

Embodiment 162 is the protein composition of embodiment 3 or any one of embodiments 4-161 as dependent on embodiment 3, comprising step (h).

Embodiment 163 is the protein composition of embodiment 162, further comprising repeating step (h).

Embodiment 164 is the protein composition of embodiment 163, wherein in the repeat of step (h), the wash solvent is the same as in the first step (h).

Embodiment 165 is the protein composition of embodiment 163, wherein in the repeat of step (h), the wash solvent is different than in the first step (h).

Embodiment 166 is the protein composition of embodiment 3 or any one of embodiments 4-165 as dependent on embodiment 3, comprising step (i).

Embodiment 167 is the protein composition of embodiment 3 or any one of embodiments 4-166 as dependent on embodiment 3, further comprising drying the protein composition.

Embodiment 168 is the protein composition of embodiment 167, comprising spray drying, mat drying, freeze-drying, or oven drying.

Embodiment 169 is the protein composition of embodiment 3 or any one of embodiments 4-168 as dependent on embodiment 3, wherein the source protein composition is at least 90% plant, algae, fungi, bacteria, protozoans, invertebrates, a part or derivative of any thereof, or a combination thereof on a dry weight basis.

Embodiment 170 is the protein composition of embodiment 169, wherein the source protein composition is at least 90% a defatted soy flour, a defatted pea flour, or a combination thereof on a dry weight basis.

Embodiment 171 is the protein composition of embodiment 3 or any one of embodiments 4-170 as dependent on embodiment 3, wherein the source protein composition is a soy protein composition, and the protein composition has an isoflavone content less than 90% of the isoflavone content of the source protein composition, on a dry weight basis.

Embodiment 172 is the protein composition of embodiment 3 or any one of embodiments 4-171 as dependent on embodiment 3, wherein the source protein composition is a soy protein composition, and the protein composition has an isoflavone content less than 70% of the isoflavone content of the source protein composition, on a dry weight basis.

Embodiment 173 is the protein composition of embodiment 3 or any one of embodiments 4-172 as dependent on embodiment 3, wherein the source protein composition is a soy protein composition, and the protein composition has an isoflavone content less than 50% of the isoflavone content of the source protein composition, on a dry weight basis.

Embodiment 174 is the protein composition of embodiment 3 or any one of embodiments 4-173 as dependent on embodiment 3, wherein the source protein composition is a soy protein composition, and the protein composition has an isoflavone content less than 30% of the isoflavone content of the source protein composition, on a dry weight basis.

Embodiment 175 is the protein composition of embodiment 3 or any one of embodiments 4-174 as dependent on embodiment 3, wherein the source protein composition is a soy protein composition, and the protein composition has an isoflavone content less than 10% of the isoflavone content of the source protein composition, on a dry weight basis.

Embodiment 176 is the protein composition of embodiment 3 or any one of embodiments 4-175 as dependent on embodiment 3, wherein, when cooked in water, a 1% (w/v) suspension of the protein composition by dry weight of the protein composition produces no more than 90% of the amount of one or more soy flavor compounds produced by cooking a 1% (w/v) suspension of the source protein composition (by dry weight of the source protein composition).

Embodiment 177 is the protein composition of embodiment 3 or any one of embodiments 4-176 as dependent on embodiment 3, wherein, when cooked in water, a 1% (w/v) suspension of the protein composition by dry weight of the protein composition produces no more than 70% of the amount of one or more soy flavor compounds produced by cooking a 1% (w/v) suspension of the source protein composition (by dry weight of the source protein composition).

Embodiment 178 is the protein composition of embodiment 3 or any one of embodiments 4-177 as dependent on embodiment 3, wherein, when cooked in water, a 1% (w/v) suspension of the protein composition by dry weight of the protein composition produces no more than 50% of the amount of one or more soy flavor compounds produced by cooking a 1% (w/v) suspension of the source protein composition (by dry weight of the source protein composition).

Embodiment 179 is the protein composition of embodiment 3 or any one of embodiments 4-178 as dependent on embodiment 3, wherein, when cooked in water, a 1% (w/v) suspension of the protein composition by dry weight of the protein composition produces no more than 30% of the amount of one or more soy flavor compounds produced by cooking a 1% (w/v) suspension of the source protein composition (by dry weight of the source protein composition).

Embodiment 180 is the protein composition of embodiment 3 or any one of embodiments 4-179 as dependent on embodiment 3, wherein, when cooked in water, a 1% (w/v) suspension of the protein composition by dry weight of the protein composition produces no more than 10% of the amount of one or more soy flavor compounds produced by cooking a 1% (w/v) suspension of the source protein composition (by dry weight of the source protein composition).

Embodiment 181 is the protein composition of any one of embodiments 176-180, wherein the one or more soy flavor compounds comprise at least one compound selected from the group consisting of hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal.

Embodiment 182 is the protein composition of embodiment 3 or any one of embodiments 4-181 as dependent on embodiment 3, wherein, when cooked in water, a 1% (w/v) suspension of the protein composition by dry weight of the protein composition produces no more than 90% of the amount of one or more volatile compounds in a set of volatile compounds produced by cooking a 1% (w/v) suspension of the source protein composition (by dry weight of the source protein composition).

Embodiment 183 is the protein composition of embodiment 3 or any one of embodiments 4-182 as dependent on embodiment 3, wherein, when cooked in water, a 1% (w/v) suspension of the protein composition by dry weight of the protein composition produces no more than 70% of the amount of one or more volatile compounds in a set of volatile compounds produced by cooking a 1% (w/v) suspension of the source protein composition (by dry weight of the source protein composition).

Embodiment 184 is the protein composition of embodiment 3 or any one of embodiments 4-183 as dependent on embodiment 3, wherein, when cooked in water, a 1% (w/v) suspension of the protein composition by dry weight of the protein composition produces no more than 50% of the amount of one or more volatile compounds in a set of volatile compounds produced by cooking a 1% (w/v) suspension of the source protein composition (by dry weight of the source protein composition).

Embodiment 185 is the protein composition of embodiment 3 or any one of embodiments 4-184 as dependent on embodiment 3, wherein, when cooked in water, a 1% (w/v) suspension of the protein composition by dry weight of the protein composition produces no more than 30% of the amount of one or more volatile compounds in a set of volatile compounds produced by cooking a 1% (w/v) suspension of the source protein composition (by dry weight of the source protein composition).

Embodiment 186 is the protein composition of embodiment 3 or any one of embodiments 4-185 as dependent on embodiment 3, wherein, when cooked in water, a 1% (w/v) suspension of the protein composition by dry weight of the protein composition produces no more than 10% of the amount of one or more volatile compounds in a set of volatile compounds produced by cooking a 1% (w/v) suspension of the source protein composition (by dry weight of the source protein composition).

Embodiment 187 is the protein composition of embodiment 3 or any one of embodiments 4-186 as dependent on embodiment 3, wherein the protein composition produces no more than 90% of the amount of one or more volatile compounds in a set of volatile compounds produced by the source protein composition by solvent-assisted flavor extraction (SAFE).

Embodiment 188 is the protein composition of embodiment 3 or any one of embodiments 4-187 as dependent on embodiment 3, wherein the protein composition produces no more than 70% of the amount of one or more volatile compounds in a set of volatile compounds produced by the source protein composition by SAFE.

Embodiment 189 is the protein composition of embodiment 3 or any one of embodiments 4-188 as dependent on embodiment 3, wherein the protein composition produces no more than 50% of the amount of one or more volatile compounds in a set of volatile compounds produced by the source protein composition by SAFE.

Embodiment 190 is the protein composition of embodiment 3 or any one of embodiments 4-189 as dependent on embodiment 3, wherein the protein composition produces no more than 30% of the amount of one or more volatile compounds in a set of volatile compounds produced by the source protein composition by SAFE.

Embodiment 191 is the protein composition of embodiment 3 or any one of embodiments 4-190 as dependent on embodiment 3, wherein the protein composition produces no more than 10% of the amount of one or more volatile compounds in a set of volatile compounds produced by the source protein composition by SAFE.

Embodiment 192 is the protein composition of any one of embodiments 182-191, wherein the set of volatile compounds comprises a volatile compound in any one of volatile sets 1-10.

Embodiment 193 is the protein composition of any one of embodiments 182-191, wherein the set of volatile compounds is any one of volatile sets 1-10.

Embodiment 194 is the protein composition of any one of embodiments 182-191, wherein the set of volatile compounds is selected from the group consisting of volatile set 1, volatile set 2, volatile set 3, volatile set 4, volatile set 5, volatile set 6, volatile set 7, volatile set 8, volatile set 9, volatile set 10, and combinations thereof.

Embodiment 195 is the protein composition of embodiment 3 or any one of embodiments 4-194 as dependent on embodiment 3, wherein the protein composition has a saponin content that is less than 50% of the saponin content of the source protein composition.

Embodiment 196 is the protein composition of embodiment 3 or any one of embodiments 4-195 as dependent on embodiment 3, wherein the protein composition has a saponin content that is less than 30% of the saponin content of the source protein composition.

Embodiment 197 is the protein composition of embodiment 3 or any one of embodiments 4-196 as dependent on embodiment 3, wherein the protein composition has a saponin content that is less than 10% of the saponin content of the source protein composition.

Embodiment 198 is the protein composition of embodiment 3 or any one of embodiments 4-197 as dependent on embodiment 3, wherein the protein composition has an isoflavone content that is less than 50% of the isoflavone content of the source protein composition.

Embodiment 199 is the protein composition of embodiment 3 or any one of embodiments 4-198 as dependent on embodiment 3, wherein the protein composition has an isoflavone content that is less than 30% of the isoflavone content of the source protein composition.

Embodiment 200 is the protein composition of embodiment 3 or any one of embodiments 4-199 as dependent on embodiment 3, wherein the protein composition has an isoflavone content that is less than 10% of the isoflavone content of the source protein composition.

Embodiment 201 is the protein composition of embodiment 3 or any one of embodiments 4-200 as dependent on embodiment 3, wherein the protein composition has a phospholipid content that is less than 50% of the phospholipid content of the source protein composition.

Embodiment 202 is the protein composition of embodiment 3 or any one of embodiments 4-201 as dependent on embodiment 3, wherein the protein composition has a phospholipid content that is less than 30% of the phospholipid content of the source protein composition.

Embodiment 203 is the protein composition of embodiment 3 or any one of embodiments 4-202 as dependent on embodiment 3, wherein the protein composition has a phospholipid content that is less than 10% of the phospholipid content of the source protein composition.

Embodiment 204 is the protein composition of embodiment 3 or any one of embodiments 4-203 as dependent on embodiment 3, wherein the protein composition has a lipid content that is less than 50% of the lipid content of the source protein composition.

Embodiment 205 is the protein composition of embodiment 3 or any one of embodiments 4-204 as dependent on embodiment 3, wherein the protein composition has a lipid content that is less than 30% of the lipid content of the source protein composition.

Embodiment 206 is the protein composition of embodiment 3 or any one of embodiments 4-205 as dependent on embodiment 3, wherein the protein composition has a lipid content that is less than 10% of the lipid content of the source protein composition.

Embodiment 207 is the protein composition of embodiment 3 or any one of embodiments 4-206 as dependent on embodiment 3, wherein the protein composition has a phospholipid content that is less than 50% of the phospholipid content of the source protein composition.

Embodiment 208 is the protein composition of embodiment 3 or any one of embodiments 4-207 as dependent on embodiment 3, wherein the protein composition has a phospholipid content that is less than 30% of the phospholipid content of the source protein composition.

Embodiment 209 is the protein composition of embodiment 3 or any one of embodiments 4-208 as dependent on embodiment 3, wherein the protein composition has a phospholipid content that is less than 10% of the phospholipid content of the source protein composition.

Embodiment 210 is the protein composition of embodiment 3 or any one of embodiments 4-209 as dependent on embodiment 3, wherein the protein composition has a phenolic acid content that is less than 50% of the phenolic acid content of the source protein composition.

Embodiment 211 is the protein composition of embodiment 3 or any one of embodiments 4-210 as dependent on embodiment 3, wherein the protein composition has a phenolic acid content that is less than 30% of the phenolic acid content of the source protein composition.

Embodiment 212 is the protein composition of embodiment 3 or any one of embodiments 4-211 as dependent on embodiment 3, wherein the protein composition has a phenolic acid content that is less than 10% of the phenolic acid content of the source protein composition.

Embodiment 213 is the protein composition of embodiment 3 or any one of embodiments 4-212 as dependent on embodiment 3, wherein the protein composition has a flavor compounds content that is less than 50% of the flavor compounds content of the source protein composition, wherein the flavor compounds are selected from the group consisting of elected from aldehydes, ketones, esters, alcohols, pyrazines, pyranones, acids, sulfur compounds, terpenes, furans, alkanes, alkenes, and combinations thereof.

Embodiment 214 is the protein composition of embodiment 3 or any one of embodiments 4-213 as dependent on embodiment 3, wherein the protein composition has a flavor compounds content that is less than 30% of the flavor compounds content of the source protein composition, wherein the flavor compounds are selected from the group consisting of elected from aldehydes, ketones, esters, alcohols, pyrazines, pyranones, acids, sulfur compounds, terpenes, furans, alkanes, alkenes, and combinations thereof.

Embodiment 215 is the protein composition of embodiment 3 or any one of embodiments 4-214 as dependent on embodiment 3, wherein the protein composition has a flavor compounds content that is less than 10% of the flavor compounds content of the source protein composition, wherein the flavor compounds are selected from the group consisting of elected from aldehydes, ketones, esters, alcohols, pyrazines, pyranones, acids, sulfur compounds, terpenes, furans, alkanes, alkenes, and combinations thereof.

Embodiment 216 is a food product comprising the protein composition of any one of embodiments 1-215.

Embodiment 217 is the food product of embodiment 216, wherein the food product is a meat substitute.

Embodiment 218 is the food product of embodiment 216, wherein the food product is a beverage.

Embodiment 219 is the food product of embodiment 218, wherein the beverage is a milk replica.

Embodiment 220 is a method for producing a protein composition, the method comprising:
(a) adding an aqueous solution to a source protein composition to form a solution of solubilized protein;
(b) optionally removing solids from the solution of solubilized protein;
(c) optionally heating the solution of solubilized protein;
(d) optionally adjusting the pH of the solution of solubilized protein to about 4.0 to about 9.0;
(e) optionally cooling the solution of solubilized protein to about 0° C. to about 10° C.;
(f) adding an organic solvent to the solution of solubilized protein to form a solid phase and a liquid phase;
(g) separating the solid phase from the liquid phase to form the protein composition;
(h) optionally washing the protein composition with a wash solvent; and
(i) optionally resolubilizing the protein composition,
wherein the protein composition comprises at least at least 50% by dry weight of a plurality of plant proteins, fungal proteins, algal proteins, bacterial proteins, protozoan proteins, invertebrate proteins.

Embodiment 221 is the method of embodiment 220, wherein the source protein composition comprises one or more toxins in an amount sufficient to harm a human being.

Embodiment 222 is the method of any one of embodiments 220 or embodiment 221, wherein the source protein composition is a cottonwood source protein composition.

Embodiment 223 is the method of any one of embodiments 220-222, wherein the source protein composition comprises gossypol in an amount of more than 450 ppm.

Embodiment 224 is the method of embodiment 223, wherein the detoxified protein composition comprises gossypol in an amount of less than 450 ppm.

Embodiment 225 is the method of embodiment 223, wherein the detoxified protein composition comprises gossypol in an amount of less than 300 ppm.

Embodiment 226 is the method of embodiment 223, wherein the detoxified protein composition comprises gossypol in an amount of less than 100 ppm.

Embodiment 227 is the method of embodiment 223, wherein the detoxified protein composition comprises gossypol in an amount of less than 10 ppm.

Embodiment 228 is the method of any one of embodiments 220-227, wherein the protein composition is the protein composition of any one of embodiments 1-215.

Embodiment 229 is the method of any one of embodiments 221-228, wherein step (a) is performed at a pH of about 7.0 to about 10.0.

Embodiment 230 is the method of any one of embodiments 220-229, wherein step (a) is performed at a pH of about 6.0 to about 9.0.

Embodiment 231 is the method of any one of embodiments 220-230, wherein step (a) is performed at a pH of about 7.5 to about 8.5.

Embodiment 232 is the method of any one of embodiments 220-231, wherein step (b) comprises centrifugation, filtration, or a combination thereof.

Embodiment 233 is the method of any one of embodiments 220-232, wherein step (d) comprises adjusting the pH of the solution of solubilized protein to about 4.0 to about 6.0.

Embodiment 234 is the method of any one of embodiments 220-233, wherein step (d) comprises adjusting the pH of the solution of solubilized protein to about 6.0 to about 7.0.

Embodiment 235 is the method of any one of embodiments 220-234, wherein step (f) comprises adding the organic solvent to a final concentration of about 5% to about 70% (v/v).

Embodiment 236 is the method of any one of embodiments 220-235, wherein step (f) comprises adding the organic solvent to a final concentration of about 10% to about 50% (v/v).

Embodiment 237 is the method of any one of embodiments 220-236, wherein step (f) comprises adding the organic solvent to a final concentration of about 40% to about 70% (v/v).

Embodiment 238 is the method of any one of embodiments 220-237, wherein at the beginning of step (f), the organic solvent has a temperature of about -20° C. to about 10° C.

Embodiment 239 is the method of any one of embodiments 220-238, wherein at the beginning of step (f), the organic solvent has a temperature of about -20° C. to about 0° C.

Embodiment 240 is the method of any one of embodiments 220-239, wherein at the beginning of step (f), the organic solvent has a temperature of about 0° C. to about 4° C.

Embodiment 241 is the method of any one of embodiments 220-240, wherein at the beginning of step (f), the organic solvent has a temperature of about 10° C. to about 25° C.

Embodiment 242 is the method of any one of embodiments 220-241, wherein step (e) comprises cooling the solution of solubilized protein to a temperature of about 0° C. to about 4° C.

Embodiment 243 is the method of any one of embodiments 220-242, wherein at the beginning of step (f), the solution of solubilized protein has a temperature of about 10° C. to about 25° C.

Embodiment 244 is the method of any one of embodiments 220-243, wherein step (c) comprises heating the solution of solubilized protein for a period of about 10 seconds to about 30 minutes.

Embodiment 245 is the method of any one of embodiments 220-244, wherein step (c) comprises heating the solution of solubilized protein for a period of about 1 minute to about 20 minutes.

Embodiment 246 is the method of any one of embodiments 220-245, wherein step (c) comprises heating the solution of solubilized protein at a temperature of about 70° C. to about 100° C.

Embodiment 247 is the method of any one of embodiments 220-246, wherein step (c) comprises heating the solution of solubilized protein at a temperature of about 85° C. to about 95° C.

Embodiment 248 is the method of any one of embodiments 220-247, wherein step (g) comprises centrifugation, filtration, or a combination thereof.

Embodiment 249 is the method of any one of embodiments 220-248, wherein the organic solvent is selected from the group consisting of ethanol, methanol, propanol, isopropyl alcohol, and acetone.

Embodiment 250 is the method of any one of embodiments 220-249, wherein the organic solvent is ethanol.

Embodiment 251 is the method of any one of embodiments 220-250, wherein the wash solvent is an organic wash solvent.

Embodiment 252 is the method of embodiment 251, wherein the organic wash solvent is the same as the organic solvent in step (f).

Embodiment 253 is the method of embodiment 251, wherein the organic wash solvent is selected from the group consisting of ethanol, methanol, propanol, isopropyl alcohol, and acetone.

Embodiment 254 is the method of embodiment 251, wherein the organic wash solvent is ethanol.

Embodiment 255 is the method of any one of embodiments 220-250, wherein the wash solvent is an aqueous solution.

Embodiment 256 is the method of any one of embodiments 220-250, wherein the wash solvent is a mixture of an aqueous solution and an organic wash solvent.

Embodiment 257 is the method of embodiment 256, wherein the wash solvent comprises about 1% to about 30% (v/v) of the organic wash solvent.

Embodiment 258 is the method of embodiment 256, wherein the wash solvent comprises about 30% to about 80% of the organic wash solvent.

Embodiment 259 is the method of embodiment 256, wherein the wash solvent comprises about 80% to about 99% of the organic wash solvent.

Embodiment 260 is the method of any one of embodiments 256-259, wherein the organic wash solvent is ethanol.

Embodiment 261 is the method of any one of embodiments 256-259, wherein the organic wash solvent in step (h) is the same as the organic solvent in step (f).

Embodiment 262 is the method of any one of embodiments 220-261, wherein the treating comprises resolubilizing the protein composition to a concentration of about 1.5 to about 50 mg/mL.

Embodiment 263 is the method of any one of embodiments 220-262, wherein the treating comprises resolubilizing the protein composition to a concentration of about 2 to about 4 mg/mL.

Embodiment 264 is the method of any one of embodiments 220-262, wherein the treating comprises resolubilizing the protein composition to a concentration of about 20 to about 40 mg/mL.

Embodiment 265 is the method of any one of embodiments 220-264, wherein the treating comprises resolubilizing at least a portion of the protein composition at a pH of at least 8.0.

Embodiment 266 is the method of embodiment 265, wherein the treating comprises resolubilizing at least a portion of the protein composition at a pH of at least 9.0.

Embodiment 267 is the method of embodiment 266, wherein the treating comprises resolubilizing at least a portion of the protein composition at a pH of at least 10.0.

Embodiment 268 is the method of any one of embodiments 265-267, further comprising neutralizing or acidifying the protein composition.

Embodiment 269 is the method of any one of embodiments 220-268, wherein the treating comprises resolubilizing at least a portion of the protein composition using an enzyme.

Embodiment 270 is the method of embodiment 266, wherein the enzyme is a protein deamidase.

Embodiment 271 is the method of embodiment 266, wherein the enzyme is a protein glutaminase.

Embodiment 272 is the method of embodiment 266, wherein the enzyme is a protein asparaginase.

Embodiment 273 is the method of any one of embodiments 220-272 comprising steps (a), (b), (f), and (g).

Embodiment 274 is the method of any one of embodiments 220-273 comprising steps (a), (b), (c), (f), and (g).

Embodiment 275 is the method of embodiment 274, wherein step (c) follows step (b).

Embodiment 276 is the method of embodiment 274, wherein step (b) follows step (c).

Embodiment 277 is the method of any one of embodiments 220-276 comprising steps (a), (b), (d), (f), and (g).

Embodiment 278 is the method of embodiment 277, wherein step (d) follows step (b).

Embodiment 279 is the method of any one of embodiments 220-278 comprising steps (a), (b), (e), (f), and (g).

Embodiment 280 is the method of embodiment 279, wherein step (e) follows step (b).

Embodiment 281 is the method of embodiment 279, wherein step (b) follows step (e).

Embodiment 282 is the method of any one of embodiments 220-282 comprising steps (a), (b), (c), (d), (f), and (g).

Embodiment 283 is the method of embodiment 282, wherein steps (b), (c), and (d) are performed in the order of (b), (c), (d).

Embodiment 284 is the method of embodiment 282, wherein steps (b), (c), and (d) are performed in the order of (c), (b), (d).

Embodiment 285 is the method of embodiment 282, wherein steps (b), (c), and (d) are performed in the order of (b), (d), (c).

Embodiment 286 is the method of any one of embodiments 220-285 comprising steps (a), (b), (c), (e), (f), and (g).

Embodiment 287 is the method of embodiment 286, wherein steps (b), (c), and (e) are performed in the order of (b), (c), (e).

Embodiment 288 is the method of embodiment 286, wherein steps (b), (c), and (e) are performed in the order of (c), (b), (e).

Embodiment 289 is the method of embodiment 286, wherein steps (b), (c), and (e) are performed in the order of (b), (e), (c).

Embodiment 290 is the method of any one of embodiments 220-289 comprising steps (a), (b), (c), (d), (e), (f), and (g).

Embodiment 291 is the method of embodiment 290, wherein steps (b), (c), (d), and (e) are performed in the order of (b), (c), (d), (e).

Embodiment 292 is the method of embodiment 290, wherein steps (b), (c), (d), and (e) are performed in the order of (c), (b), (d), (e).

Embodiment 293 is the method of embodiment 290, wherein steps (b), (c), (d), and (e) are performed in the order of (b), (d), (e), (c).

Embodiment 294 is the method of embodiment 290, wherein steps (b), (c), (d), and (e) are performed in the order of (b), (d), (c), (e).

Embodiment 295 is the method of any one of embodiments 220-294 comprising steps (a), (c), (f), and (g).

Embodiment 296 is the method of any one of embodiments 220-295 comprising steps (a), (c), (d), (f), and (g).

Embodiment 297 is the method of embodiment 296, wherein step (c) is performed before step (d).

Embodiment 298 is the method of embodiment 296, wherein step (d) is performed before step (c).

Embodiment 299 is the method of any one of embodiments 220-298 comprising steps (a), (c), (d), (e), (f), and (g).

Embodiment 300 is the method of embodiment 299, wherein steps (c), (d), and (e) are performed in the order (c), (d), (e).

Embodiment 301 is the method of embodiment 299, wherein steps (c), (d), and (e) are performed in the order (d), (e), (c).

Embodiment 302 is the method of embodiment 299, wherein steps (c), (d), and (e) are performed in the order (d), (c), (e).

Embodiment 303 is the method of any one of embodiments 220-302 comprising steps (a), (d), (f), and (g).

Embodiment 304 is the method of any one of embodiments 220-303 comprising steps (a), (d), (e), (f), and (g).

Embodiment 305 is the method of embodiment 220, wherein step (d) is performed before step (e).

Embodiment 306 is the method of any one of embodiments 220-305, comprising steps (a), (e), (f), and (g).

Embodiment 307 is the method of any one of embodiments 220-306, comprising step (h).

Embodiment 308 is the method of embodiment 306, further comprising repeating step (h).

Embodiment 309 is the method of embodiment 308, wherein in the repeat of step (h), the wash solvent is the same as in the first step (h).

Embodiment 310 is the method of embodiment 308, wherein in the repeat of step (h), the wash solvent is different than in the first step (h).

Embodiment 311 is the method of any one of embodiments 220-310, comprising step (i).

Embodiment 312 is the method of any one of embodiments 220-311 as dependent on embodiment 3, further comprising drying the protein composition.

Embodiment 313 is the method of embodiment 312, comprising spray drying, mat drying, freeze-drying, or oven drying.

Embodiment 314 is the method of any one of embodiments 220-313, wherein the source protein composition is at least 90% plant, algae, fungi, bacteria, protozoans, invertebrates, a part or derivative of any thereof, or a combination thereof on a dry weight basis.

Embodiment 315 is the method of embodiment 314, wherein the source protein composition is at least 90% a defatted soy flour, a defatted pea flour, or a combination thereof on a dry weight basis.

Embodiment 316 is the method of any one of embodiments 220-315, wherein the source protein composition is a soy protein composition, and the protein composition has an isoflavone content less than 90% of the isoflavone content of the source protein composition, on a dry weight basis.

Embodiment 317 is the method of any one of embodiments 220-316, wherein the source protein composition is a soy protein composition, and the protein composition has an isoflavone content less than 70% of the isoflavone content of the source protein composition, on a dry weight basis.

Embodiment 318 is the method of any one of embodiments 220-317, wherein the source protein composition is a soy protein composition, and the protein composition has an isoflavone content less than 50% of the isoflavone content of the source protein composition, on a dry weight basis.

Embodiment 319 is the method of any one of embodiments 220-318, wherein the source protein composition is a soy protein composition, and the protein composition has an isoflavone content less than 30% of the isoflavone content of the source protein composition, on a dry weight basis.

Embodiment 320 is the method of any one of embodiments 220-319, wherein the source protein composition is a soy protein composition, and the protein composition has an isoflavone content less than 10% of the isoflavone content of the source protein composition, on a dry weight basis.

Embodiment 321 is the method of any one of embodiments 220-320, wherein, when cooked in water, a 1% (w/v) suspension of the protein composition by dry weight of the protein composition produces no more than 90% of the amount of one or more soy flavor compounds produced by cooking a 1% (w/v) suspension of the source protein composition (by dry weight of the source protein composition).

Embodiment 322 is the method of any one of embodiments 220-321, wherein, when cooked in water, a 1% (w/v) suspension of the protein composition by dry weight of the protein composition produces no more than 70% of the amount of one or more soy flavor compounds produced by cooking a 1% (w/v) suspension of the source protein composition (by dry weight of the source protein composition).

Embodiment 323 is the method of any one of embodiments 220-322, wherein, when cooked in water, a 1% (w/v) suspension of the protein composition by dry weight of the protein composition produces no more than 50% of the amount of one or more soy flavor compounds produced by cooking a 1% (w/v) suspension of the source protein composition (by dry weight of the source protein composition).

Embodiment 324 is the method of any one of embodiments 220-323, wherein, when cooked in water, a 1% (w/v) suspension of the protein composition by dry weight of the protein composition produces no more than 30% of the amount of one or more soy flavor compounds produced by cooking a 1% (w/v) suspension of the source protein composition (by dry weight of the source protein composition).

Embodiment 325 is the method of any one of embodiments 220-323, wherein, when cooked in water, a 1% (w/v) suspension of the protein composition by dry weight of the protein composition produces no more than 10% of the amount of one or more soy flavor compounds produced by cooking a 1% (w/v) suspension of the source protein composition (by dry weight of the source protein composition).

Embodiment 326 is the method of any one of embodiments 321-325, wherein the one or more soy flavor compounds comprise at least one compound selected from the group consisting of hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal.

Embodiment 327 is the method of any one of embodiments 220-326, wherein, when cooked in water, a 1% (w/v) suspension of the protein composition by dry weight of the protein composition produces no more than 90% of the amount of one or more volatile compounds in a set of volatile compounds produced by cooking a 1% (w/v) suspension of the source protein composition (by dry weight of the source protein composition).

Embodiment 328 is the method of any one of embodiments 220-327, wherein, when cooked in water, a 1% (w/v) suspension of the protein composition by dry weight of the protein composition produces no more than 70% of the amount of one or more volatile compounds in a set of volatile compounds produced by cooking a 1% (w/v) suspension of the source protein composition (by dry weight of the source protein composition).

Embodiment 329 is the method of any one of embodiments 220-328, wherein, when cooked in water, a 1% (w/v) suspension of the protein composition by dry weight of the protein composition produces no more than 50% of the amount of one or more volatile compounds in a set of volatile compounds produced by cooking a 1% (w/v) suspension of the source protein composition (by dry weight of the source protein composition).

Embodiment 330 is the method of any one of embodiments 220-329, wherein, when cooked in water, a 1% (w/v) suspension of the protein composition by dry weight of the protein composition produces no more than 30% of the amount of one or more volatile compounds in a set of volatile compounds produced by cooking a 1% (w/v) suspension of the source protein composition (by dry weight of the source protein composition).

Embodiment 331 is the method of any one of embodiments 220-330, wherein, when cooked in water, a 1% (w/v) suspension of the protein composition by dry weight of the protein composition produces no more than 10% of the amount of one or more volatile compounds in a set of volatile compounds produced by cooking a 1% (w/v) suspension of the source protein composition (by dry weight of the source protein composition).

Embodiment 332 is the method of any one of embodiments 220-331, wherein the protein composition produces no more than 90% of the amount of one or more volatile compounds in a set of volatile compounds produced by the source protein composition by solvent-assisted flavor extraction (SAFE).

Embodiment 333 is the method of any one of embodiments 220-332, wherein the protein composition produces no more than 70% of the amount of one or more volatile compounds in a set of volatile compounds produced by the source protein composition by SAFE.

Embodiment 334 is the method of any one of embodiments 220-333, wherein the protein composition produces no more than 50% of the amount of one or more volatile compounds in a set of volatile compounds produced by the source protein composition by SAFE.

Embodiment 335 is the method of any one of embodiments 220-334, wherein the protein composition produces no more than 30% of the amount of one or more volatile compounds in a set of volatile compounds produced by the source protein composition by SAFE.

Embodiment 336 is the method of any one of embodiments 220-335, wherein the protein composition produces no more than 10% of the amount of one or more volatile compounds in a set of volatile compounds produced by the source protein composition by SAFE.

Embodiment 337 is the method of any one of embodiments 327-336, wherein the set of volatile compounds comprises a volatile compound in any one of volatile sets 1-10.

Embodiment 338 is the method of any one of embodiments 327-336, wherein the set of volatile compounds is any one of volatile sets 1-10.

Embodiment 339 is the method of any one of embodiments 327-336, wherein the set of volatile compounds is selected from the group consisting of volatile set 1, volatile set 2, volatile set 3, volatile set 4, volatile set 5, volatile set 6, volatile set 7, volatile set 8, volatile set 9, volatile set 10, and combinations thereof.

Embodiment 340 is the method of any one of embodiments 220-339, wherein the protein composition has a saponin content that is less than 50% of the saponin content of the source protein composition.

Embodiment 341 is the method of any one of embodiments 220-340, wherein the protein composition has a saponin content that is less than 30% of the saponin content of the source protein composition.

Embodiment 342 is the method of any one of embodiments 220-341, wherein the protein composition has a saponin content that is less than 10% of the saponin content of the source protein composition.

Embodiment 343 is the method of any one of embodiments 220-342, wherein the protein composition has an isoflavone content that is less than 50% of the isoflavone content of the source protein composition.

Embodiment 344 is the method of any one of embodiments 220-343, wherein the protein composition has an isoflavone content that is less than 30% of the isoflavone content of the source protein composition.

Embodiment 345 is the method of any one of embodiments 220-344, wherein the protein composition has an isoflavone content that is less than 10% of the isoflavone content of the source protein composition.

Embodiment 346 is the method of any one of embodiments 220-345, wherein the protein composition has a phospholipid content that is less than 50% of the phospholipid content of the source protein composition.

Embodiment 347 is the method of any one of embodiments 220-346, wherein the protein composition has a phospholipid content that is less than 30% of the phospholipid content of the source protein composition.

Embodiment 348 is the method of any one of embodiments 220-347, wherein the protein composition has a phospholipid content that is less than 10% of the phospholipid content of the source protein composition.

Embodiment 349 is the method of any one of embodiments 220-348, wherein the protein composition has a lipid content that is less than 50% of the lipid content of the source protein composition.

Embodiment 350 is the method of any one of embodiments 220-349, wherein the protein composition has a lipid content that is less than 30% of the lipid content of the source protein composition.

Embodiment 351 is the method of any one of embodiments 220-350, wherein the protein composition has a lipid content that is less than 10% of the lipid content of the source protein composition.

Embodiment 352 is the method of any one of embodiments 220-351, wherein the protein composition has a phosphatidylcholine content that is less than 50% of the phosphatidylcholine content of the source protein composition.

Embodiment 353 is the method of any one of embodiments 220-352, wherein the protein composition has a phosphatidylcholine content that is less than 30% of the phosphatidylcholine content of the source protein composition.

Embodiment 354 is the method of any one of embodiments 220-353, wherein the protein composition has a phosphatidylcholine content that is less than 10% of the phosphatidylcholine content of the source protein composition.

Embodiment 355 is the method of any one of embodiments 220-354, wherein the protein composition has a phenolic acid content that is less than 50% of the phenolic acid content of the source protein composition.

Embodiment 356 is the method of any one of embodiments 220-355, wherein the protein composition has a phenolic acid content that is less than 30% of the phenolic acid content of the source protein composition.

Embodiment 357 is the method of any one of embodiments 220-356, wherein the protein composition has a phenolic acid content that is less than 10% of the phenolic acid content of the source protein composition.

Embodiment 358 is the method of any one of embodiments 220-357, wherein the protein composition has a flavor compounds content that is less than 50% of the flavor compounds content of the source protein composition, wherein the flavor compounds are selected from the group consisting of elected from aldehydes, ketones, esters, alcohols, pyrazines, pyranones, acids, sulfur compounds, terpenes, furans, alkanes, alkenes, and combinations thereof.

Embodiment 359 is the method of any one of embodiments 220-358, wherein the protein composition has a flavor compounds content that is less than 30% of the flavor compounds content of the source protein composition, wherein the flavor compounds are selected from the group consisting of elected from aldehydes, ketones, esters, alcohols, pyrazines, pyranones, acids, sulfur compounds, terpenes, furans, alkanes, alkenes, and combinations thereof.

Embodiment 360 is the method of any one of embodiments 220-359, wherein the protein composition has a flavor compounds content that is less than 10% of the flavor compounds content of the source protein composition, wherein the flavor compounds are selected from the group consisting of elected from aldehydes, ketones, esters, alcohols, pyrazines, pyranones, acids, sulfur compounds, terpenes, furans, alkanes, alkenes, and combinations thereof.

Embodiment 361 is a food product comprising a protein composition produced by the method of any one of embodiments 220-360.

Embodiment 362 is the food product of embodiment 361, wherein the food product is a meat substitute.

Embodiment 363 is the food product of embodiment 361, wherein the food product is a beverage.

Embodiment 364 is the food product of embodiment 361, wherein the beverage is a milk replica.

Embodiment 365 is a method of extracting small molecules from a protein source composition, the method comprising:
(a) adding an aqueous solution to a source protein composition to form a solution of solubilized protein;
(b) optionally removing solids from the solution of solubilized protein;
(c) optionally heating the solution of solubilized protein;
(d) optionally adjusting the pH of the solution of solubilized protein to about 4.0 to about 9.0;
(e) optionally cooling the solution of solubilized protein to about 0° C. to about 10° C.;
(f) adding an organic solvent to the solution of solubilized protein to form a solid phase and a liquid phase;
(g) separating the solid phase from the liquid phase to form a solution enriched in small molecules.

Embodiment 366 is the method of embodiment 365, wherein the source protein composition is a soy source protein composition.

Embodiment 367 is the method of embodiment 366, wherein the solution enriched in small molecules comprises isoflavones.

Embodiment 368 is the method of any one of embodiments 365-367, wherein step (a) is performed at a pH of about 7.0 to about 10.0.

Embodiment 369 is the method of any one of embodiments 365-368, wherein step (a) is performed at a pH of about 6.0 to about 9.0.

Embodiment 370 is the method of any one of embodiments 365-369, wherein step (a) is performed at a pH of about 7.5 to about 8.5.

Embodiment 371 is the method of any one of embodiments 365-370, wherein step (b) comprises centrifugation, filtration, or a combination thereof.

Embodiment 372 is the method of any one of embodiments 365-371, wherein step (d) comprises adjusting the pH of the solution of solubilized protein to about 4.0 to about 6.0.

Embodiment 373 is the method of any one of embodiments 365-372, wherein step (d) comprises adjusting the pH of the solution of solubilized protein to about 6.0 to about 7.0.

Embodiment 374 is the method of any one of embodiments 365-373, wherein step (f) comprises adding the organic solvent to a final concentration of about 5% to about 70% (v/v).

Embodiment 375 is the method of any one of embodiments 365-374, wherein step (f) comprises adding the organic solvent to a final concentration of about 10% to about 50% (v/v).

Embodiment 376 is the method of any one of embodiments 365-374, wherein step (f) comprises adding the organic solvent to a final concentration of about 40% to about 70% (v/v).

Embodiment 377 is the method of any one of embodiments 365-376, wherein at the beginning of step (f), the organic solvent has a temperature of about -20° C. to about 10° C.

Embodiment 378 is the method of any one of embodiments 365-377, wherein at the beginning of step (f), the organic solvent has a temperature of about -20° C. to about 0° C.

Embodiment 379 is the method of any one of embodiments 365-377, wherein at the beginning of step (f), the organic solvent has a temperature of about 0° C. to about 4° C.

Embodiment 380 is the method of any one of embodiments 365-379, wherein at the beginning of step (f), the organic solvent has a temperature of about 10° C. to about 25° C.

Embodiment 381 is the method of any one of embodiments 365-380, wherein step (e) comprises cooling the solution of solubilized protein to a temperature of about 0° C. to about 4° C.

Embodiment 382 is the method of any one of embodiments 365-380, wherein at the beginning of step (f), the solution of solubilized protein has a temperature of about 10° C. to about 25° C.

Embodiment 383 is the method of any one of embodiments 365-382, wherein step (c) comprises heating the solution of solubilized protein for a period of about 10 seconds to about 30 minutes.

Embodiment 384 is the method of any one of embodiments 365-383, wherein step (c) comprises heating the solution of solubilized protein for a period of about 1 minute to about 20 minutes.

Embodiment 385 is the method of any one of embodiments 365-384, wherein step (c) comprises heating the solution of solubilized protein at a temperature of about 70° C. to about 100° C.

Embodiment 386 is the method of any one of embodiments 365-385, wherein step (c) comprises heating the solution of solubilized protein at a temperature of about 85° C. to about 95° C.

Embodiment 387 is the method of any one of embodiments 365-386, wherein step (g) comprises centrifugation, filtration, or a combination thereof.

Embodiment 388 is the method of any one of embodiments 365-387, wherein the organic solvent is selected from the group consisting of ethanol, methanol, propanol, isopropyl alcohol, and acetone.

Embodiment 389 is the method of any one of embodiments 365-388, wherein the organic solvent is ethanol.

Embodiment 390 is the method of any one of embodiments 365-389, wherein the wash solvent is an organic wash solvent.

Embodiment 391 is the method of embodiment 390, wherein the organic wash solvent is the same as the organic solvent in step (f).

Embodiment 392 is the method of embodiment 390, wherein the organic wash solvent is selected from the group consisting of ethanol, methanol, propanol, isopropyl alcohol, and acetone.

Embodiment 393 is the method of embodiment 390, wherein the organic wash solvent is ethanol.

Embodiment 394 is the method of any one of embodiments 365-388, wherein the wash solvent is an aqueous solution.

Embodiment 395 is the method of any one of embodiments 365-388, wherein the wash solvent is a mixture of an aqueous solution and an organic wash solvent.

Embodiment 396 is the method of embodiment 395, wherein the wash solvent comprises about 1% to about 30% (v/v) of the organic wash solvent.

Embodiment 397 is the method of embodiment 395, wherein the wash solvent comprises about 30% to about 80% of the organic wash solvent.

Embodiment 398 is the method of embodiment 395, wherein the wash solvent comprises about 80% to about 99% of the organic wash solvent.

Embodiment 399 is the method of any one of embodiments 395-398, wherein the organic wash solvent is ethanol.

Embodiment 400 is the method of any one of embodiments 395-398, wherein the organic wash solvent in step (h) is the same as the organic solvent in step (f).

Embodiment 401 is the method of any one of embodiments 365-400, comprising steps (a), (b), (f), and (g).

Embodiment 402 is the method of any one of embodiments 365-401, comprising steps (a), (b), (c), (f), and (g).

Embodiment 403 is the method of embodiment 402, wherein step (c) follows step (b).

Embodiment 404 is the method of embodiment 402, wherein step (b) follows step (c).

Embodiment 405 is the method of any one of embodiments 365-404, comprising steps (a), (b), (d), (f), and (g).

Embodiment 406 is the method of embodiment 405, wherein step (d) follows step (b).

Embodiment 407 is the method of any one of embodiments 365-406, comprising steps (a), (b), (e), (f), and (g).

Embodiment 408 is the method of embodiment 407, wherein step (e) follows step (b).

Embodiment 409 is the method of embodiment 407, wherein step (b) follows step (e).

Embodiment 410 is the method of any one of embodiments 365-409, comprising steps (a), (b), (c), (d), (f), and (g).

Embodiment 411 is the method of embodiment 410, wherein steps (b), (c), and (d) are performed in the order of (b), (c), (d).

Embodiment 412 is the method of embodiment 410, wherein steps (b), (c), and (d) are performed in the order of (c), (b), (d).

Embodiment 413 is the method of embodiment 410, wherein steps (b), (c), and (d) are performed in the order of (b), (d), (c).

Embodiment 414 is the method of any one of embodiments 365-413, comprising steps (a), (b), (c), (e), (f), and (g).

Embodiment 415 is the method of embodiment 414, wherein steps (b), (c), and (e) are performed in the order of (b), (c), (e).

Embodiment 416 is the method of embodiment 414, wherein steps (b), (c), and (e) are performed in the order of (c), (b), (e).

Embodiment 417 is the method of embodiment 414, wherein steps (b), (c), and (e) are performed in the order of (b), (e), (c).

Embodiment 418 is the method of any one of embodiments 365-417, comprising steps (a), (b), (c), (d), (e), (f), and (g).

Embodiment 419 is the method of embodiment 418, wherein steps (b), (c), (d), and (e) are performed in the order of (b), (c), (d), (e).

Embodiment 420 is the method of embodiment 418, wherein steps (b), (c), (d), and (e) are performed in the order of (c), (b), (d), (e).

Embodiment 421 is the method of embodiment 418, wherein steps (b), (c), (d), and (e) are performed in the order of (b), (d), (e), (c).

Embodiment 422 is the method of embodiment 418, wherein steps (b), (c), (d), and (e) are performed in the order of (b), (d), (c), (e).

Embodiment 423 is the method of any one of embodiments 365-422, comprising steps (a), (c), (f), and (g).

Embodiment 424 is the method of any one of embodiments 365-422, comprising steps (a), (c), (d), (f), and (g).

Embodiment 425 is the method of embodiment 424, wherein step (c) is performed before step (d).

Embodiment 426 is the method of embodiment 424, wherein step (d) is performed before step (c).

Embodiment 427 is the method of any one of embodiments 365-426, comprising steps (a), (c), (d), (e), (f), and (g).

Embodiment 428 is the method of embodiment 427, wherein steps (c), (d), and (e) are performed in the order (c), (d), (e).

Embodiment 429 is the method of embodiment 427, wherein steps (c), (d), and (e) are performed in the order (d), (e), (c).

Embodiment 430 is the method of embodiment 427, wherein steps (c), (d), and (e) are performed in the order (d), (c), (e).

Embodiment 431 is the method of any one of embodiments 365-430, comprising steps (a), (d), (f), and (g).

Embodiment 432 is the method of any one of embodiments 365-430, comprising steps (a), (d), (e), (f), and (g).

Embodiment 433 is the method of embodiment 365, wherein step (d) is performed before step (e).

Embodiment 434 is the method of any one of embodiments 365-433, comprising steps (a), (e), (f), and (g).

Embodiment 435 is a food product comprising:
a fat;
optionally one or more flavor precursor compounds; and
at least 10% by dry weight of a protein composition, wherein the protein composition is the protein composition of any one of embodiments 1-215.

Embodiment 436 is a food product comprising:
a fat;
optionally one or more flavor precursor compounds; and
at least 10% by dry weight of a protein composition, wherein the protein composition is a protein composition produced by the method of any one of embodiments 220-360.

Embodiment 437 is the food product of any one of embodiments 435-436, wherein the food product is a plant-based food product.

Embodiment 438 is the food product of any one of embodiments 435-436, wherein the food product is an algae-based food product.

Embodiment 439 is the food product of any one of embodiments 435-436, wherein the food product is a fungus-based food product.

Embodiment 440 is the food product of any one of embodiments 435-436, wherein the food product is an invertebrate-based food product.

Embodiment 441 is the food product of any one of embodiments 435-440, wherein the food product is a meat replica.

Embodiment 442 is the food product of embodiment 441, wherein the food product is in the form of ground meat, a sausage, or a cut of meat.

Embodiment 443. The food product of any one of embodiments 435-442, wherein the food product is plant-based.

Embodiment 444 is the food product of any one of embodiments 435-443, wherein the food product contains less than 10% by weight animal products.

Embodiment 445 is the food product of any one of embodiments 435-444, wherein the food product contains less than 5% by weight animal products.

Embodiment 446 is the food product of any one of embodiments 435-445, wherein the food product contains less than 1% by weight animal products.

Embodiment 447 is the food product of any one of embodiments 435-446, wherein the food product contains no animal products.

Embodiment 448 is the food product of any one of embodiments 435-447, wherein the fat comprises at least one fat selected from the group consisting of corn oil, olive oil, soy oil, peanut oil, walnut oil, almond oil, sesame oil, cottonseed oil, rapeseed oil, canola oil, safflower oil, sunflower oil, flax seed oil, palm oil, palm kernel oil, coconut oil, babassu oil, shea butter, mango butter, cocoa butter, wheat germ oil, rice bran oil, and combinations thereof.

Embodiment 449 is the food product of any one of embodiments 435-448, wherein the one or more flavor precursors comprise at least one compound selected from the group consisting of glucose, ribose, cysteine, a cysteine derivative, thiamine, alanine, methionine, lysine, a lysine derivative, glutamic acid, a glutamic acid derivative, IMP, GMP, lactic acid, maltodextrin, creatine, alanine, arginine, asparagine, aspartate, glutamic acid, glutamine, glycine, histidine, isoleucine, leucine, methionine, phenylalanine, proline, threonine, tryptophan, tyrosine, valine, linoleic acid, and mixtures thereof.

Embodiment 450 is the food product of any one of embodiments 435-449, wherein the fat is present in the food product in an amount of about 5% to about 80% by dry weight of the food product.

Embodiment 451 is the food product of any one of embodiments 435-450, wherein the fat is present in the food product in an amount of about 10% to about 30% by dry weight of the food product.

Embodiment 452 is the food product of any one of embodiments 435-451, further comprising about 0.01% to about 5% by dry weight of a heme-containing protein.

Embodiment 453 is the food product of any one of embodiments 435-451, further comprising about 0.01% to about 7% by dry weight of a heme-containing protein.

Embodiment 454 is the food product of any one of embodiments 435-453, wherein the food product is a beverage.

Embodiment 455 is the food product of embodiment 454, wherein the fat is present in the food product in an amount of about 0.01% to about 5% by weight of the beverage.

Embodiment 456 is the food product of embodiment 454 or embodiment 455, wherein the beverage is a milk replica.

Embodiment 457 is a method for preparing a food product, the method comprising:

combining a fat, one or more optional flavor precursor compounds, and a protein composition, wherein the protein composition is the protein composition of any one of embodiments 1-215.

Embodiment 458 is a method for preparing a food product, the method comprising:

combining a fat, one or more optional flavor precursor compounds, and a protein composition, the protein composition produced by the method by the method of any one of embodiments 220-360.

Embodiment 459 is a method for reducing perceived protein source flavor in a food product, the method comprising:
combining a fat, one or more flavor precursor compounds and a protein composition, the protein composition produced by the method of any one of embodiments 220-360,
wherein at least 5% by weight of the protein content of the food product comprises the protein composition, thereby reducing perceived protein source flavor in a food product, as compared to a food product having a similar protein content but lacking the protein composition.

Embodiment 460 is the method of any one of embodiments 458-459, wherein the protein composition is the protein composition of any one of embodiments 1-215.

Embodiment 461 is the method of any one of embodiments 457-460, wherein the food product is a plant-based food product.

Embodiment 462 is the method of any one of embodiments 457-460, wherein the food product is an algae-based food product.

Embodiment 463 is the method of any one of embodiments 457-460, wherein the food product is a fungus-based food product.

Embodiment 464 is the method of any one of embodiments 457-460, wherein the food product is an invertebrate-based food product.

Embodiment 465 is the method of any one of embodiments 457-464, wherein the fat comprises at least one fat selected from the group consisting of corn oil, olive oil, soy oil, peanut oil, walnut oil, almond oil, sesame oil, cottonseed oil, rapeseed oil, canola oil, safflower oil, sunflower oil, flax seed oil, palm oil, palm kernel oil, coconut oil, babassu oil, shea butter, mango butter, cocoa butter, wheat germ oil, rice bran oil, and combinations thereof.

Embodiment 466 is the method of any one of embodiments 457-465, wherein the one or more flavor precursors comprise at least one compound selected from the group consisting of glucose, ribose, cysteine, a cysteine derivative, thiamine, alanine, methionine, lysine, a lysine derivative, glutamic acid, a glutamic acid derivative, IMP, GMP, lactic acid, maltodextrin, creatine, alanine, arginine, asparagine, aspartate, glutamic acid, glutamine, glycine, histidine, isoleucine, leucine, methionine, phenylalanine, proline, threonine, tryptophan, tyrosine, valine, linoleic acid, and mixtures thereof.

Embodiment 467 is a method of evaluating a protein composition for effect on flavor in a food product, the method comprising:
determining that a level of one or more volatile compounds in a set of volatile compounds of a first protein composition from a protein source is higher than the level of the one or more volatile compounds of a second protein composition from the protein source; and
determining that the second protein composition is superior to the first protein composition for use in a food product.

Embodiment 468 is a method of evaluating a protein composition for effect on flavor in a food product, the method comprising:
determining that a level of one or more volatile compounds in a set of volatile compounds of a source protein composition from a protein source is higher than the level of the one or more volatile compounds of a protein composition from the protein source; and
determining that the protein composition is superior to the source protein composition for use in a food product.

Embodiment 469 is the method of embodiment 467, wherein the second protein composition is the protein composition of any one of embodiments 1-215.

Embodiment 470 is the method of embodiment 468, wherein the protein composition is the protein composition of any one of embodiments 1-215.

Embodiment 471 is the method of any one of embodiments 467-470, wherein the food product is the food product of any one of embodiments 435-456.

Embodiment 472 is the method of any one of embodiments 467-471, wherein the set of volatile compounds comprises a volatile compound from any one of volatile sets 1-10.

Embodiment 473 is the method of any one of embodiments 467-471, wherein the set of volatile compounds is any one of volatile sets 1-10.

Embodiment 474 is the method of any one of embodiments 467-471, wherein the set of volatile compounds is selected from the group consisting of volatile set 1, volatile set 2, volatile set 3, volatile set 4, volatile set 5, volatile set 6, volatile set 7, volatile set 8, volatile set 9, volatile set 10, and combinations thereof.

Embodiment 475 is the method of any one of embodiments 467-474, wherein the protein source is a plant, a fungus, algae, bacteria, protozoa, an invertebrate, or a combination thereof.

Embodiment 476 is the method of embodiment 475, wherein the protein source is soy.

Embodiment 477 is the method of embodiment 476, wherein the set of volatile compounds comprise at least one compound selected from the group consisting of hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal.

Embodiment 478 is the method of embodiment 477, wherein the set of volatile compounds is hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal.

Embodiment 479 is the method of any one of embodiments 467-478, wherein the food product is a meat replica.

Embodiment 480 is the method of any one of embodiments 467-479, wherein the food product is plant-based.

Embodiment 481 is the method of any one of embodiments 467-480, wherein the food product contains less than 10% by weight animal products.

Embodiment 482 is the method of any one of embodiments 467-481, wherein the food product contains less than 5% by weight animal products.

Embodiment 483 is the method of any one of embodiments 467-482, wherein the food product contains less than 1% by weight animal products.

Embodiment 484 is the method of any one of embodiments 467-483, wherein the food product contains no animal products.

Embodiment 485 is a method of reducing flavor in a protein composition, the method comprising:
(a) determining a level of one or more volatile compounds in a set of volatile compounds of a first protein composition from a protein source;
(b) preparing a second protein composition from the protein source, wherein preparing the second protein composition comprises reducing the amount of one or more components of the protein source that are included in the second protein composition; and
(c) determining that a level of one or more volatile compounds in a set of volatile compounds from the second protein composition is lower than the level of the one or more volatile compounds in a set of volatile compounds in the first protein composition.

Embodiment 486 is a method of determining a cause of flavor in a protein composition, the method comprising:
(a) determining a level of one or more volatile compounds in a set of volatile compounds of a first protein composition from a protein source;
(b) providing a second protein composition from the protein source, wherein the second protein composition comprises a decreased amount of one or more components of the protein source;
(c) determining that a level of one or more volatile compounds in a set of volatile compounds from the second protein composition is lower than the level the of one or more volatile compounds in a set of volatile compounds in the first protein composition; and
(d) identifying the one or more components of the protein course to be a cause of flavor in the protein composition.

Embodiment 487 is the method of embodiment 485 or embodiment 486, wherein the second protein composition is the protein composition of any one of embodiments 1-215.

Embodiment 488 is the method of any one of embodiments 485-487, wherein the set of volatile compounds comprises a volatile compound from any one of volatile sets 1-10.

Embodiment 489 is the method of any one of embodiments 485-487, wherein the set of volatile compounds is any one of volatile sets 1-10.

Embodiment 490 is the method of any one of embodiments 485-489, wherein the set of volatile compounds is selected from the group consisting of volatile set 1, volatile set 2, volatile set 3, volatile set 4, volatile set 5, volatile set 6, volatile set 7, volatile set 8, volatile set 9, volatile set 10, and combinations thereof.

Embodiment 491 is the method of any one of embodiments 485-490, wherein the protein source is a plant, a fungus, algae, bacteria, protozoa, an invertebrate, or a combination thereof.

Embodiment 492 is the method of embodiment 491, wherein the protein source is soy.

Embodiment 493 is the method of embodiment 492, wherein the set of volatile compounds comprise at least one compound selected from the group consisting of hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal.

Embodiment 494 is the method of embodiment 493, wherein the set of volatile compounds is hexanal, pentanal, 2-pentylfuran, 1-octen-3-ol, 1-octen-3-one, 1-hexanol, (E)-2-nonenal, (E,Z)-2,6-nonadienal, and (E,E)-2,4-decadienal.

Embodiment 495 is the method of any one of embodiments 485-494, wherein the component of the protein source that is decreased comprises lipids.

Embodiment 496 is the method of any one of embodiments 485-495, wherein the component of the protein source that is decreased comprises a fatty acid, a wax, a sterol, a monoglyceride, a diglyceride, a triglyceride, a sphingolipid, phospholipid, or a combination thereof.

Embodiment 497 is the method of any one of embodiments 485-496, wherein the component of the protein source that is decreased comprises phospholipids.

Embodiment 498 is the method of any one of embodiments 485-497, wherein the decreased amount of one or more components of the protein source in the second protein composition is at least a 10% decrease compared to the first protein composition.

Embodiment 499 is the method of any one of embodiments 485-497, wherein the decreased amount of one or more components of the protein source in the second protein composition is at least a 30% decrease compared to the first protein composition.

Embodiment 500 is the method of any one of embodiments 485-497, wherein the decreased amount of one or more components of the protein source in the second protein composition is at least a 50% decrease compared to the first protein composition.

Embodiment 501 is the method of any one of embodiments 485-497, wherein the decreased amount of one or more components of the protein source in the second protein composition is at least a 70% decrease compared to the first protein composition.

Embodiment 502 is the method of any one of embodiments 485-497, wherein the decreased amount of one or more components of the protein source in the second protein composition is at least a 90% decrease compared to the first protein composition.

Embodiment 503 is a milk replica comprising:

an emulsion of a fat, water, and the protein composition of any one of embodiments 1-215.

Embodiment 504 is the milk replica of embodiment 503, wherein the fat is present in the milk replica in an amount of about 0.01% to about 5% of the milk replica.

Embodiment 505 is the milk replica of embodiment 504, wherein the fat is selected from the group consisting of corn oil, olive oil, soy oil, peanut oil, walnut oil, almond oil, sesame oil, cottonseed oil, rapeseed oil, canola oil, safflower oil, sunflower oil, flax seed oil, palm oil, palm kernel oil, coconut oil, babassu oil, shea butter, mango butter, cocoa butter, wheat germ oil, rice bran oil, and combinations thereof.

Embodiment 506 is the milk replica of embodiment 503 or embodiment 504, wherein the emulsion is stable when added to a liquid with a temperature of between about 50° C. to about 85° C.

Embodiment 507 is the milk replica of embodiment 505, wherein the liquid is coffee, espresso, or a combination thereof.

The materials and methods of the disclosure will be further described in the following examples, which do not limit the scope of the methods and compositions of matter described in the claims.

### EXAMPLES

### Example 1

### Prepare “PureSPI” (Feedstock Was Defatted Soy Flour)

Aqueous extraction: 100 g of defatted soy flour was added to 1 L water while stirring at 400 RPM at room temperature (RT). The pH was adjusted to 8.0 using concentrated sodium hydroxide. Stirring continued for 30 minutes at RT. The mixture was centrifuged at 3,000 x g for 3 minutes at RT before taking supernatant. The heavy phase, mainly soy fiber, was discarded.

Solvent precipitation: The supernatant, a light yellow colored slightly cloudy solution, was mixed with equal volume (0.8 L) of 200 proof ethanol. Heavy white precipitate formed. The mixture was stirred at RT for 10 minutes. The mixture was centrifuged at 3,000 x g for 3 minutes at RT. The supernatant was a light-yellow colored clear solution, with protein removed and soy isoflavones enriched.

Washing: The heavy phase from the last step was a soft off-white solid. Equal volume (0.3 L) of 200 proof ethanol was added. The mixture was stirred at RT for 10 minutes. The mixture was centrifuged at 3,000 x g for 3 minutes at RT. The supernatant, a slightly yellow-colored clear solution, was discarded.

Drying: The heavy phase from the last step was a soft white solid. It was freeze-dried and grinded into powder with a benchtop blender. This is the final soy protein isolate product, termed “pureSPI”.

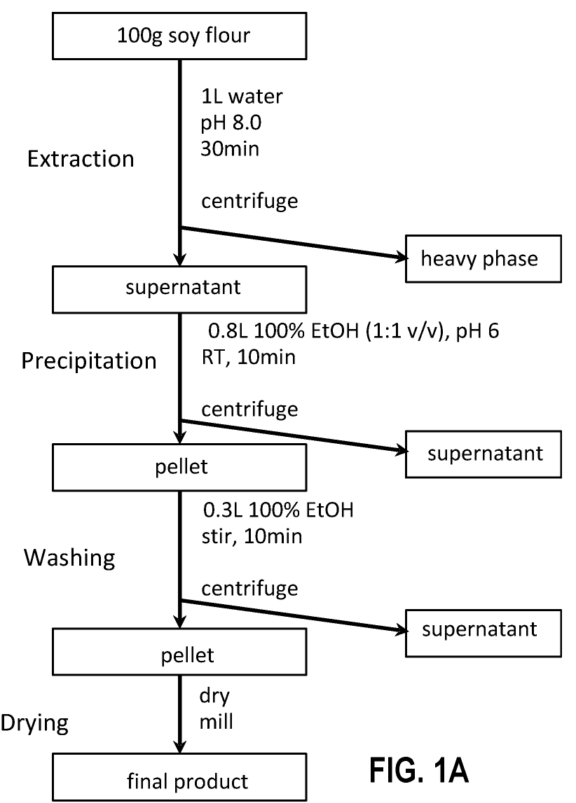

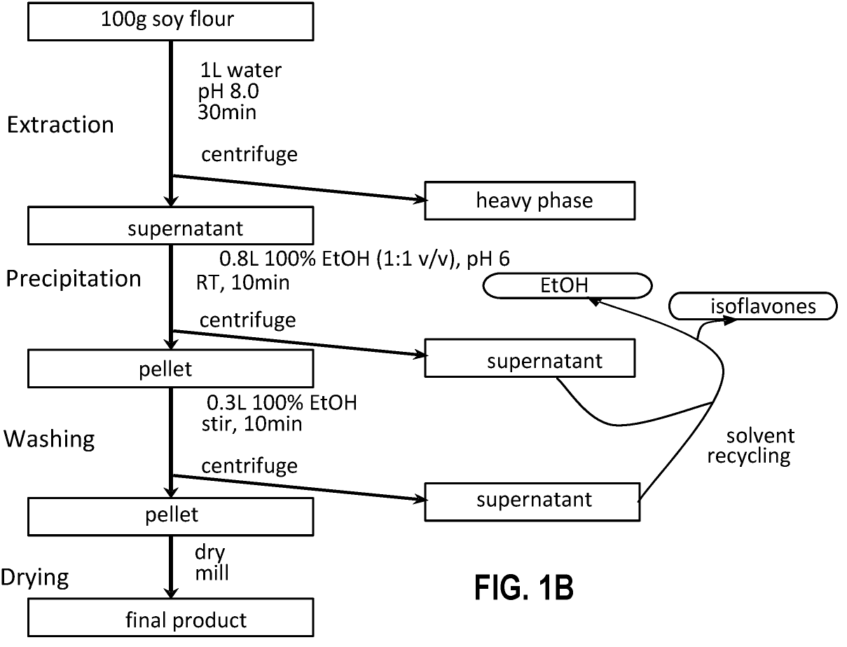

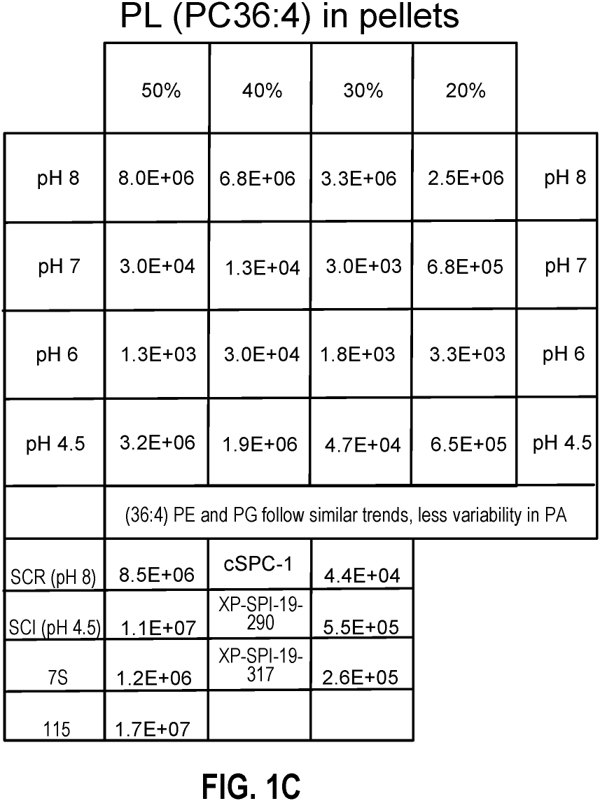

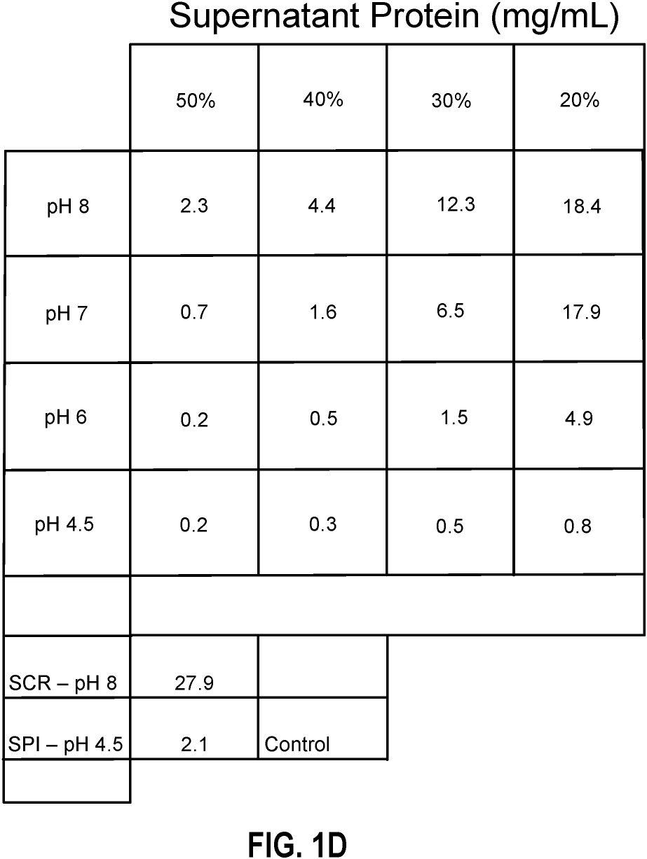

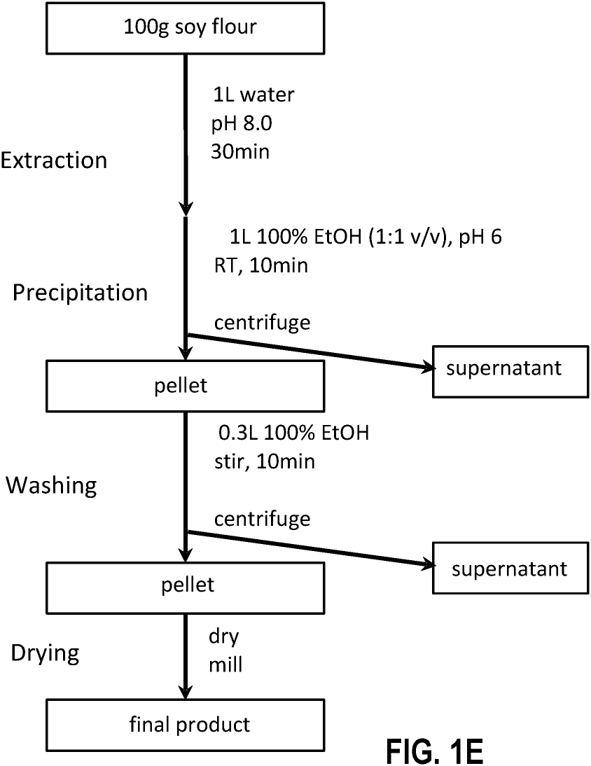

The process flowchart is shown in FIG. 1A. Another exemplary process flowchart is shown in FIG. 1B.

Exemplary phospholipid content for various protein preparation conditions is shown in FIG. 1C. Exemplary protein content in the precipitation supernatant is shown in FIG. 1D.

### Example 2

### Prepare “PureSPC” (Feedstock Is Defatted Soy Flour)

Aqueous extraction: 100 g of defatted soy flour was added to 1 L water while stirring at 400 RPM at room temperature (RT). The pH was adjusted to 8.0 using concentrated sodium hydroxide. Stirring continued for 30 minutes at RT.

Solvent precipitation: Without removal of the fiber, the extraction slurry was mixed with equal volume (1 L) of 200 proof ethanol. Heavy white precipitate formed. The mixture was stirred at RT for 10 minutes. The mixture was centrifuged at 3,000 x g for 3 minutes at RT. The supernatant was a light-yellow colored clear solution, with protein removed and soy isoflavones enriched.

Washing: The heavy phase from the last step is a soft off-white solid. Equal volume (0.6 L) of 200 proof ethanol was added. The mixture was stirred at RT for 10 minutes. The mixture was centrifuged at 3,000 x g for 3 minutes at RT. The supernatant, a slightly yellow-colored clear solution, was discarded.

Drying: The heavy phase from the last step is a soft white solid. It was freeze-dried and grinded into powder with a benchtop blender. This is the final soy protein concentrate product, termed “pureSPC”.

The process flowchart is shown in FIG. 1E.

### Example 3

### Prepare “Pureleaf” (Feedstock Is a Fresh Leafy Green Vegetable)

Aqueous extraction: 500 g of fresh spinach was added to 1.5 L pre-chilled water in a benchtop blender. The mixture was blended for 3 minutes to release leaf proteins. The mixture was centrifuged at 3,000 x g for 10 minutes at RT before taking supernatant. The heavy phase, mainly fibers, was discarded.

Solvent precipitation: The supernatant, a dark-green colored solution, was mixed with equal volume (1.8 L) of 200 proof ethanol. Heavy green precipitate formed. The mixture was stirred at RT for 10 minutes. The mixture was centrifuged at 3,000 x g for 3 minutes at RT. The supernatant was a yellowish green colored clear solution.

Washing: The heavy phase from the last step is a dark green solid. Equal volume (0.3 L) of 200 proof ethanol was added. The mixture was stirred at RT for 10 minutes. The mixture was centrifuged at 3,000 x g for 3 minutes at RT. The supernatant, a dark-green clear solution, contained most of the chlorophyll, a potential high-value byproduct from this process. The heavy phase was washed one more time with 0.3 L 200 proof ethanol to remove residual chlorophyll.

Drying: The heavy phase from the last step is a soft off-white solid. It was freeze-dried and grinded into powder with a benchtop blender. This is the final leaf protein isolate product, termed “pureLeaf”.

### Example 4

### Prepare “PurePPI” (Feedstock Is Defatted Pea Flour)

Aqueous extraction: 100 g of defatted pea flour was added to 1 L water while stirring at 400 RPM at room temperature (RT). The pH was adjusted to 8.0 using concentrated sodium hydroxide. Stirring continued for 30 minutes at RT. The mixture was centrifuged at 3,000 x g for 3 minutes at RT before taking supernatant. The heavy phase, mainly pea starch, was discarded.

Solvent precipitation: The supernatant, a light-yellow colored slightly cloudy solution, was mixed with equal volume (0.8 L) of 200 proof ethanol. Heavy white precipitate formed. The mixture was stirred at RT for 10 minutes. The mixture was centrifuged at 3,000 x g for 3 minutes at RT. The supernatant was a light-yellow colored clear solution, with protein removed.

Washing: The heavy phase from the last step is a soft off-white solid. Equal volume (0.3 L) of 200 proof ethanol was added. The mixture was stirred at RT for 10 minutes. The mixture was centrifuged at 3,000 x g for 3 minutes at RT. The supernatant, a slightly yellow-colored clear solution, was discarded.

Drying: The heavy phase from the last step is a soft white solid. It was freeze-dried and grinded into powder with a benchtop blender. This is the final pea protein isolate product, termed “purePPI”.

### Example 5

### Prepare “PureCPI” (Feedstock Is a Cottonseed Meal)

Aqueous extraction: 100 g of cottonseed presscake was added to 1 L water in a blender. The mixture was blended to homogeneity and the pH was adjusted to 8.0 using concentrated sodium hydroxide. Mixture was kept at RT for 30 minutes with occasional stirring. Fibers were removed by running the mixture through a mesh filter and a centrifugation at 3,000 x g for 3 minutes.

Solvent precipitation: The supernatant, a brown colored slightly cloudy solution, was mixed with equal volume (0.8 L) of 200 proof ethanol. Heavy precipitate formed. The mixture was stirred at RT for 10 minutes. The mixture was centrifuged at 3,000 x g for 3 minutes at RT. The supernatant was a yellow colored clear solution, with protein removed.

Washing: The heavy phase from the last step is a soft brown solid. Equal volume (0.3 L) of 200 proof ethanol was added. The mixture was stirred at RT for 10 minutes. The mixture was centrifuged at 3,000 x g for 3 minutes at RT. The supernatant, a slightly yellow-colored clear solution, was discarded.

Drying: The heavy phase from the last step is a soft off-white solid. It was freeze-dried and grinded into powder with a benchtop blender. This is the final cottonseed protein isolate product, termed “pureCPI”.

### Example 6

### Prepare “PureInsect” (Feedstock Is Whole Insects)

Aqueous extraction: 30 g of whole dried mealworms was added to 300 mL water and blended at room temperature (RT) for 3 min in a benchtop blender. Stirring continued for 30 minutes at RT. The mixture was centrifuged at 3,000 x g for 3 minutes at RT before taking supernatant. The heavy phase was discarded.

Solvent precipitation: The supernatant, a light-brown colored slightly cloudy solution, was mixed with equal volume (0.2 L) of 200 proof ethanol. Heavy white precipitate formed. The mixture was stirred at RT for 10 minutes. The mixture was centrifuged at 3,000 x g for 3 minutes at RT. The supernatant was a light-brown colored clear solution, with protein removed.

Washing: The heavy phase from the last step is a soft off-white solid. Equal volume (0.2 L) of 200 proof ethanol was added. The mixture was stirred at RT for 10 minutes. The mixture was centrifuged at 3,000 x g for 3 minutes at RT. The supernatant, a slightly yellow-colored clear solution, was discarded.

Drying: The heavy phase from the last step is a soft white solid. It was freeze-dried and grinded into powder with a benchtop blender. This is the final mealworm protein isolate product, termed “pureInsect”.

### Example 7

### GCMS Characterization

pureSPI and pureSPC were compared to commercial soy protein isolates (SPIs) and commercial soy protein concentrates (SPCs) when cooked in water. Four commercial products were used, designated as “cSPI-1” (a commercial soy protein isolate), “cSPI-2” (a commercial soy protein isolate), “cSPC-1” (a commercial soy protein concentrate), and “cSPC-2” (a commercial soy protein concentrate).

The impacts of adding plant protein ingredients into a flavor system were assessed by comparing the volatile compounds profiles with solid-phase microextraction-gas chromatography mass spectrometry (SPME/GC-MS). pureSPI was compared to two commercial soy protein isolates. 1% of the protein ingredient was added to a flavor broth (FLB) and cooked. The flavor broth contained a reducing sugar, a sulfur-containing amino acid, and a heme-containing protein. Additional controls included blank (water) and the meat flavor broth alone. All samples were prepared in quadruplicates. The volatiles of the cooked broth were analyzed on an Agilent GCMS.

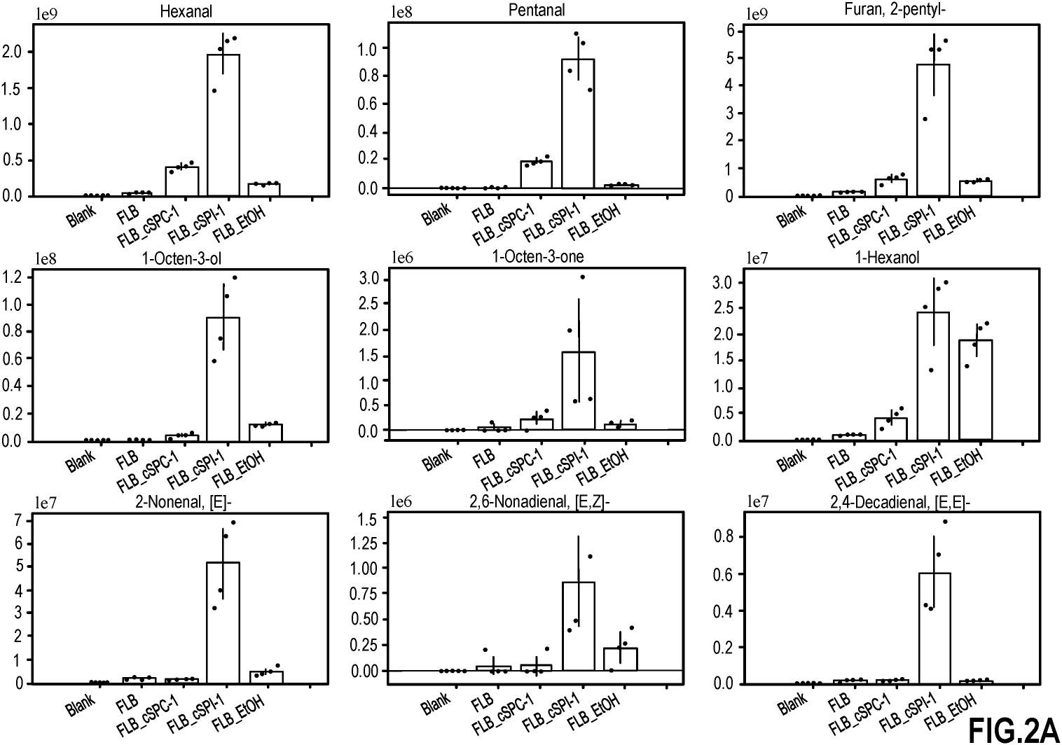

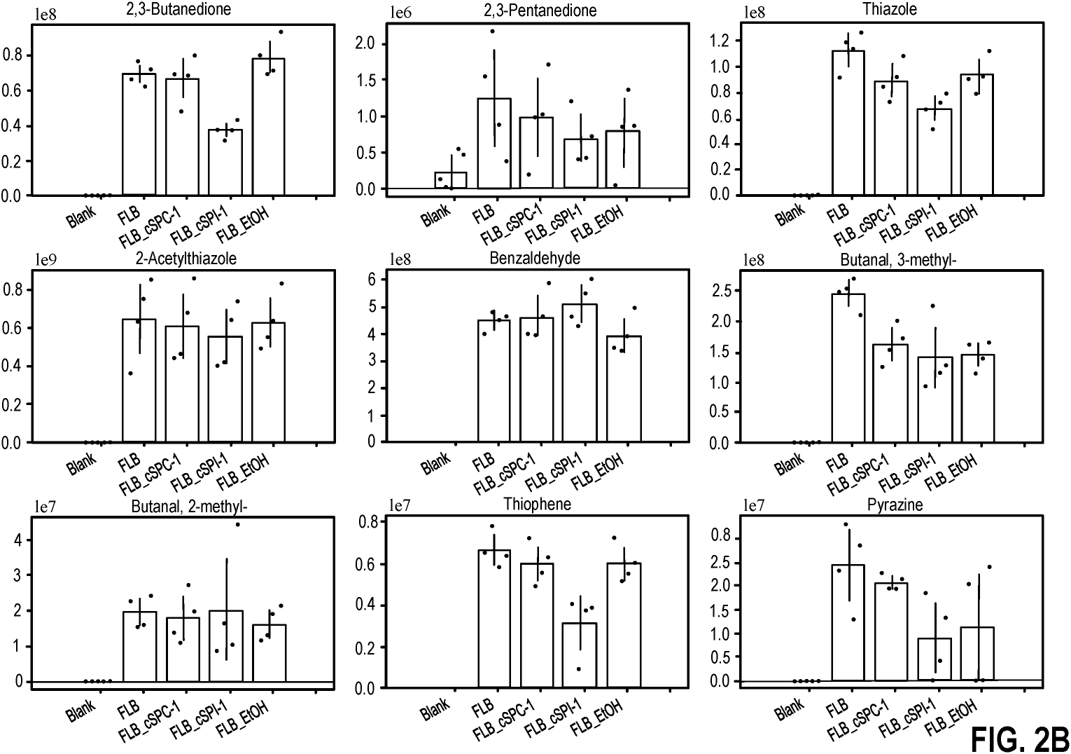

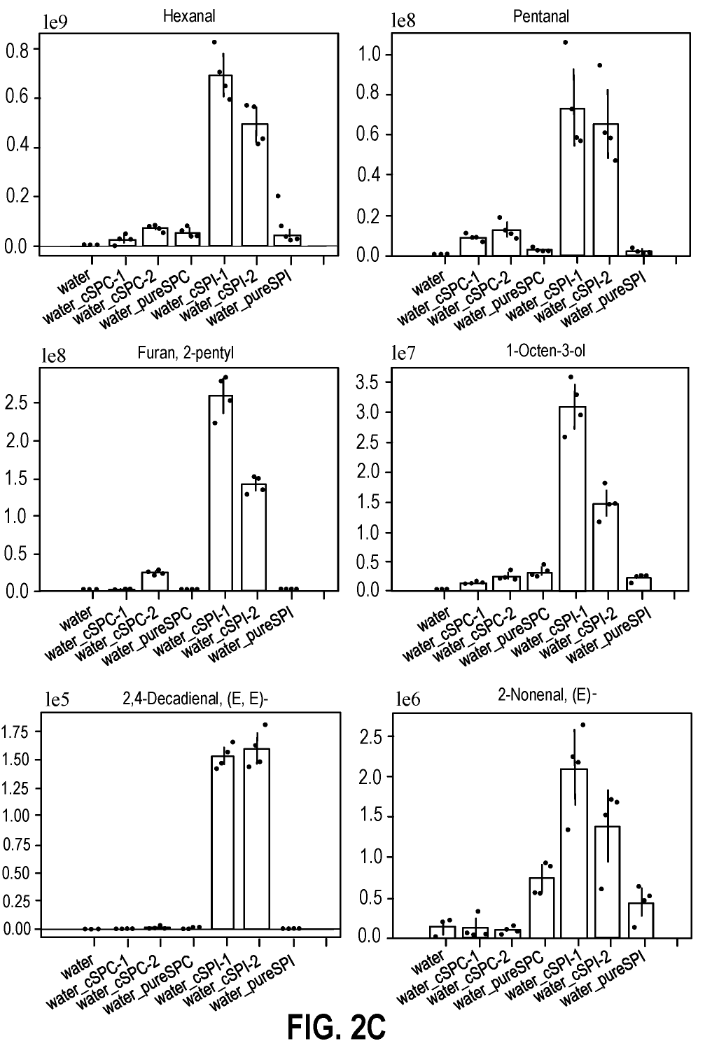

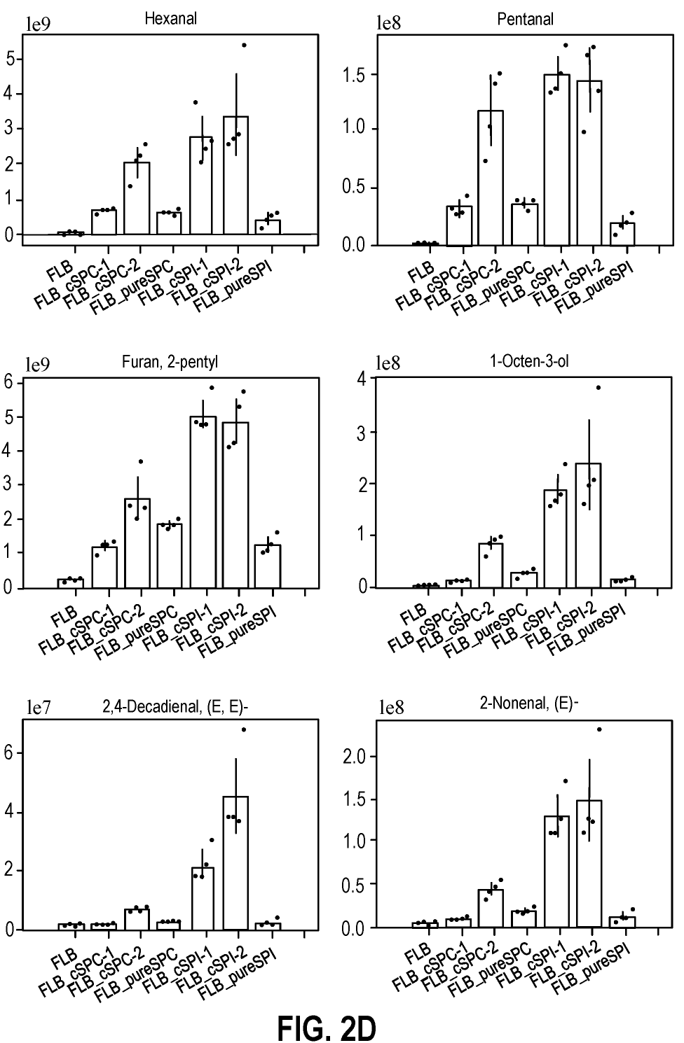

Soy flavor in these samples was assessed by comparing the GCMS peak intensity of a panel of 9 soy flavor compounds (hexanal, pentanal, 2-pentyl-furan, 1-octen-3-ol, 1-octen-3-one, 1-hexanol, 2-nonenal, 2,6-nonadienal, and 2,4-decadienal). When comparing the pureSPI samples to the commercial SPI samples, all 9 compounds showed significantly decreased peak intensities. When comparing the pureSPI samples to the commercial SPC samples, 3 compounds showed increased peak intensities, 3 compounds showed decreased peak intensities, and 3 compounds showed similar intensities. (FIG. 2A)

Meat flavor in these samples was assessed by comparing the GCMS peak intensity of a panel of 9 meat flavor compounds (2,3-butanedione, 2,3-pentanedione, thiazole, 2-acetylthiazole, benzaldehyde, 3-methyl-butanal, 2-methyl-butanal, thiophene, and pyrazine). When comparing the pureSPI samples to the commercial SPI samples, 2 compounds showed significantly increased peak intensities, and the other 7 compounds had similar intensities. When comparing the pureSPI samples to the commercial SPC samples, all 9 compounds showed similar intensities. (FIG. 2B)

These data showed that pureSPI, in the meat flavor system, generated less soy flavor and better meat flavor than the commercial SPI.

Additional exemplary data from cooking a 1% (w/v) protein suspension in water and in a flavor broth are shown in FIGS. 2C and 2D, respectively.

### Example 8

### Analyses of Soy Isoflavones

Isoflavones are a group of plant-derived phenolic compounds. These compounds have a bitter and astringent taste and also contribute to the yellow color of soy products. On the other hand, while more careful clinic studies are required, isoflavones are suggested to possess antioxidant, anticancer, antimicrobial, and anti-inflammatory properties. There is commercial value and market for soy isoflavones. Soybean has three isoflavone aglycons, namely, genistein, daidzein, and glycitein, each of which has multiple glucosidic forms (e.g., glucoside, acetylglucoside, and malonylglucoside forms). Six isoforms, genistein, daidzein, glycitein, genistin, daidzin, and glycitin were quantified from the in-process samples of the pureSPI process.

Of the total isoflavones (sum of 6 isoforms) from the starting material, 56.3% were present in the ethanol precipitation supernatant, 18.3% were present in the wash supernatant, and 4.2% were present in the pureSPI final product. These data support that 1) pureSPI process efficiently removed >95% of the isoflavones from the protein fraction, which contributes to the better flavor and better color of the final product; 2) More than 70% of isoflavones were extracted in the ethanol waste streams (precipitation supernatant and the wash supernatant), which could be recovered during ethanol recycling.

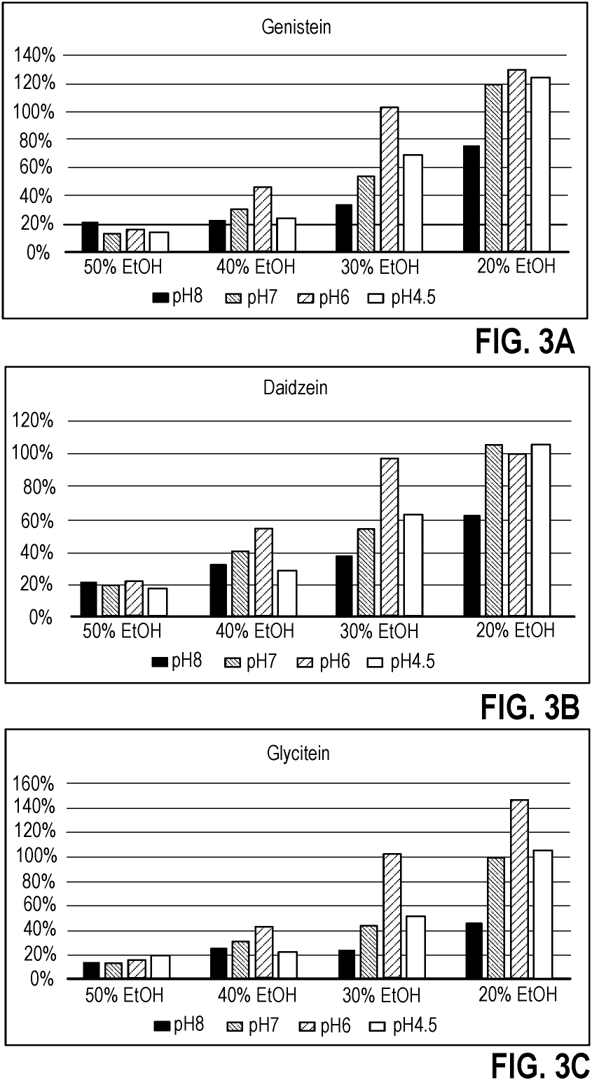

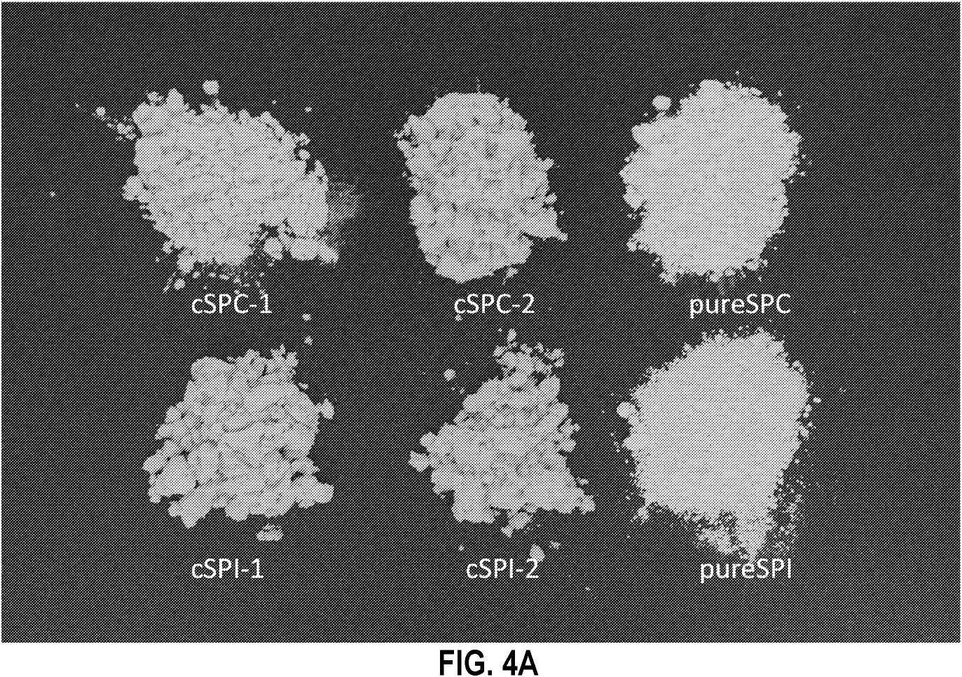

Exemplary data for genistein, daidzein, and glycitein under various protein preparation conditions are shown in FIGS. 3A-C.

### Example 9

### Analyses of Gossypol in Cottonseed Protein

Gossypol is a phenolic compound present in cottonseed. High concentrations of free gossypol are toxic and limit the use of cottonseed applications as human food. U.S. federal regulation requires free gossypol content not to exceed 450 parts per million (ppm) when using cottonseed products for human consumption (21 C.F.R. 172.894). The gossypol contents were quantified from the in-process samples of the pureCPI process as described in Example 5.

Most of the gossypol was removed during the process. <1.0 ppm free gossypol was detected in the final pureCPI product.

### Example 10

### Color Characterization

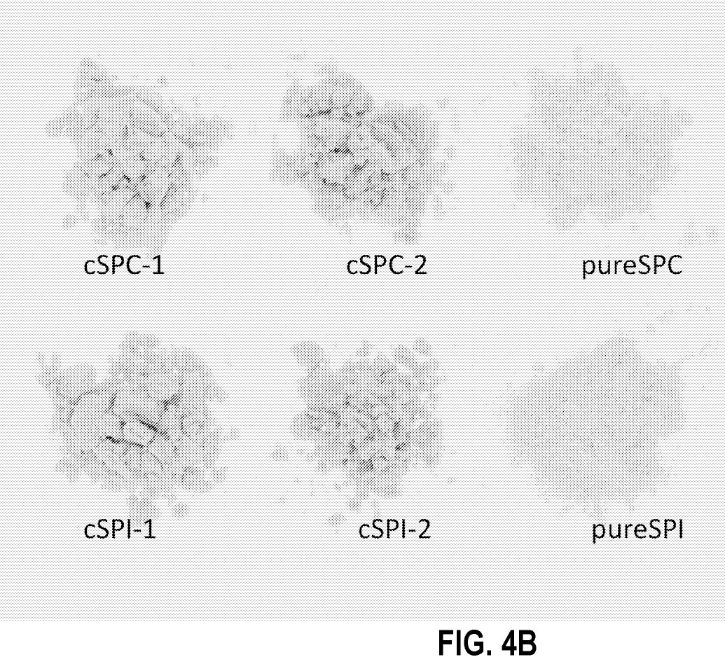

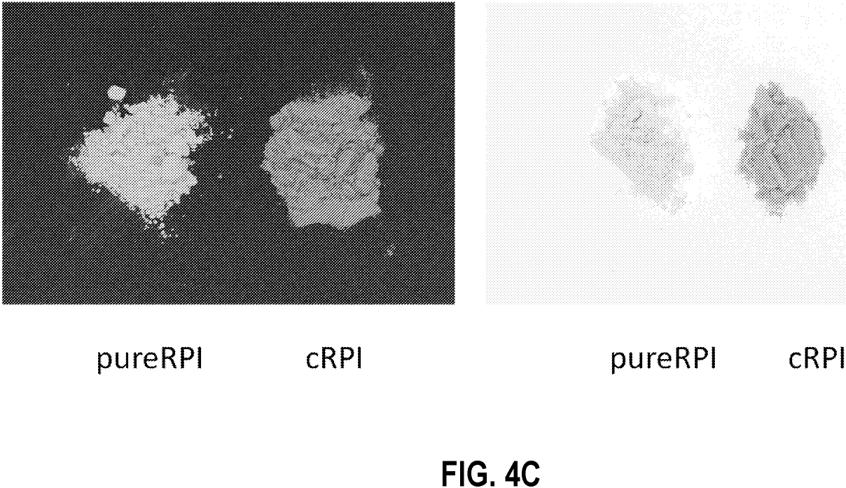

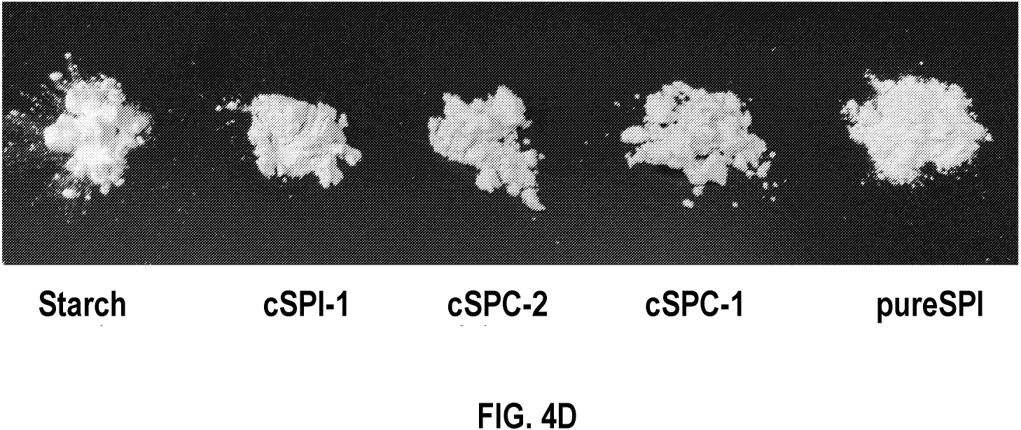

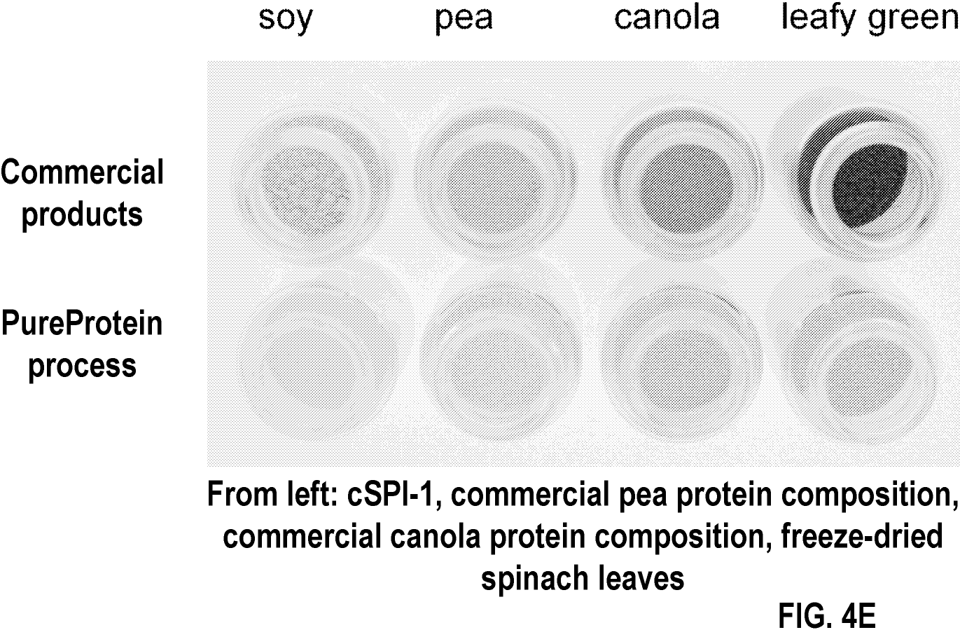

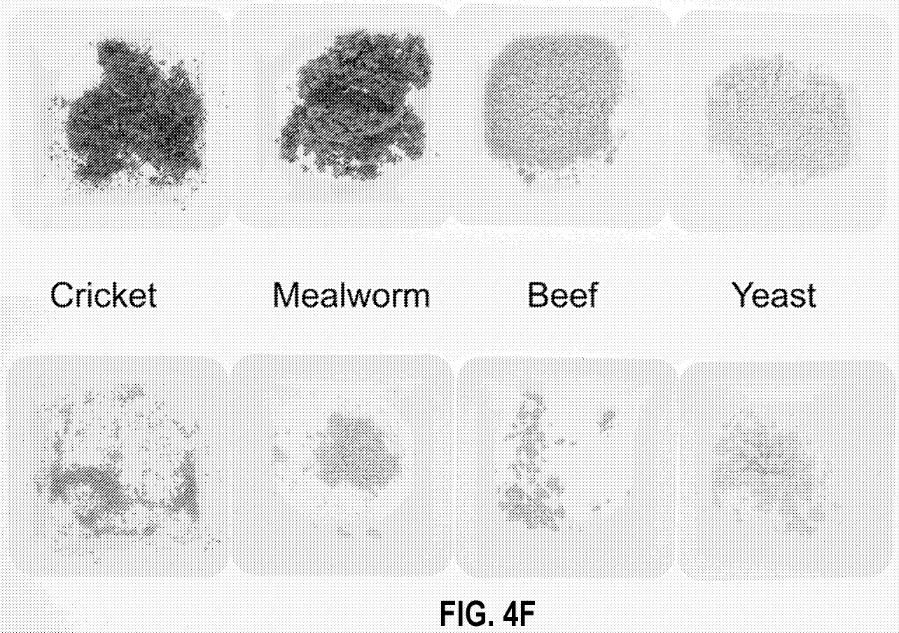

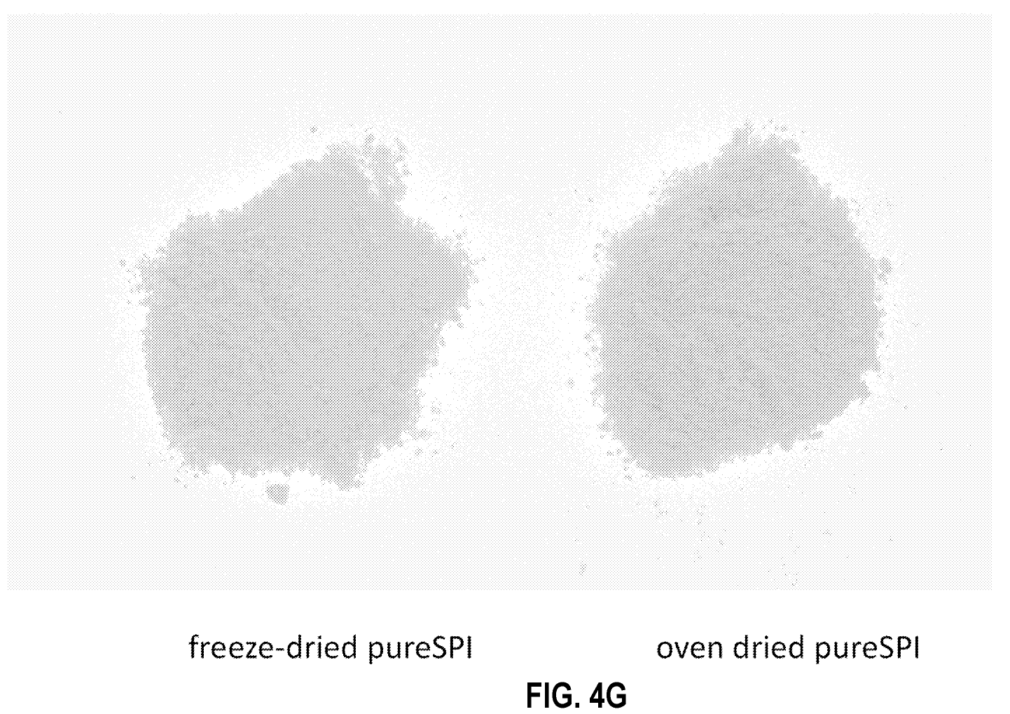

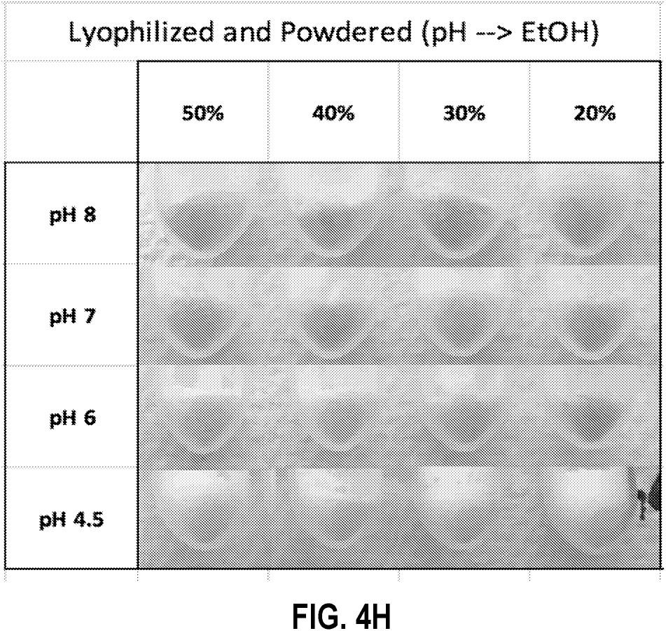

Final products from the pureProtein process have a desirable bright white color. A visual difference in color was observed when compared to exemplary commercial soy protein contenders, as shown in FIGS. 4A-D. FIGS. 4E and 4F shows exemplary improvement in color from various protein sources, including soy, peas, canola, leafy greens, crickets, mealworms, beef, and yeast. In FIG. 4G, the same preparation of pureSPI was dried either by a lyophilizer or by an oven at 80° C., showing that the pureSPI process is compatible with multiple drying methods. FIG. 4H shows exemplary differences in color when different protein preparation conditions are used

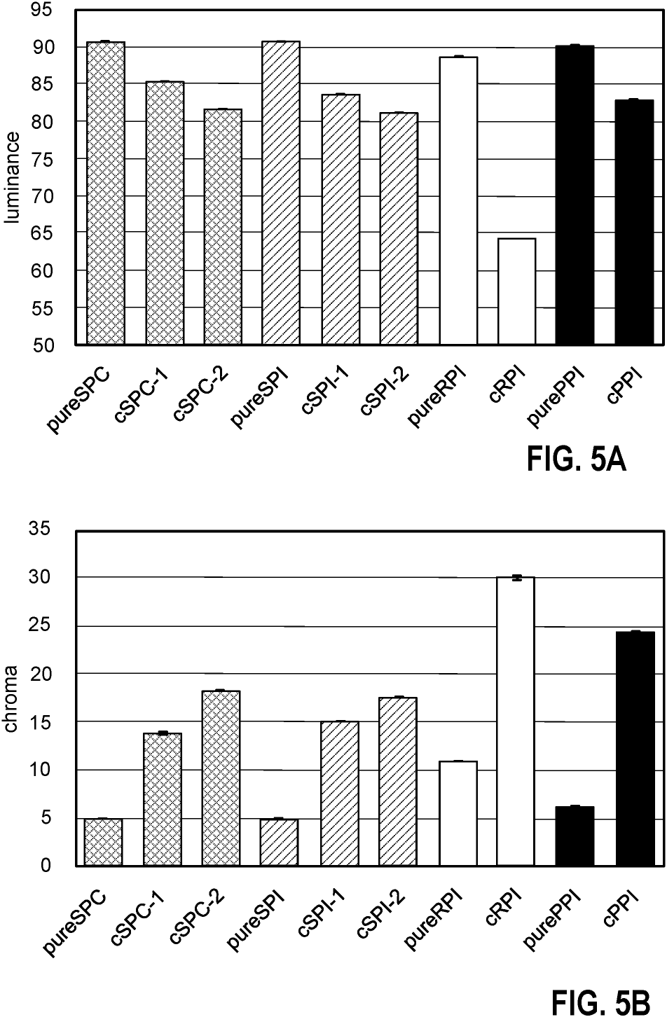

FIGS. 5A and 5B show exemplary color characterization. FIG. 5A shows luminance data, and FIG. 5B shows chroma data, as determined on a chroma meter. A) Each of pureSPC, pureSPI, pureRPI, and purePPI has a higher luminance value and thus brighter than their commercial contenders. B) Each of pureSPC, pureSPI, pureRPI, and purePPI has a lower chroma value and thus less colorful than their commercial contenders.

### Example 11

### Sensory Characterization of the Ground Meat Application

The impacts of adding soy protein ingredients into a meat analog product were assessed by a sensory panel. pureSPI was compared to commercial soy protein isolate (cSPI-1) and commercial soy protein concentrates (cSPC-1 and cSPC-2).

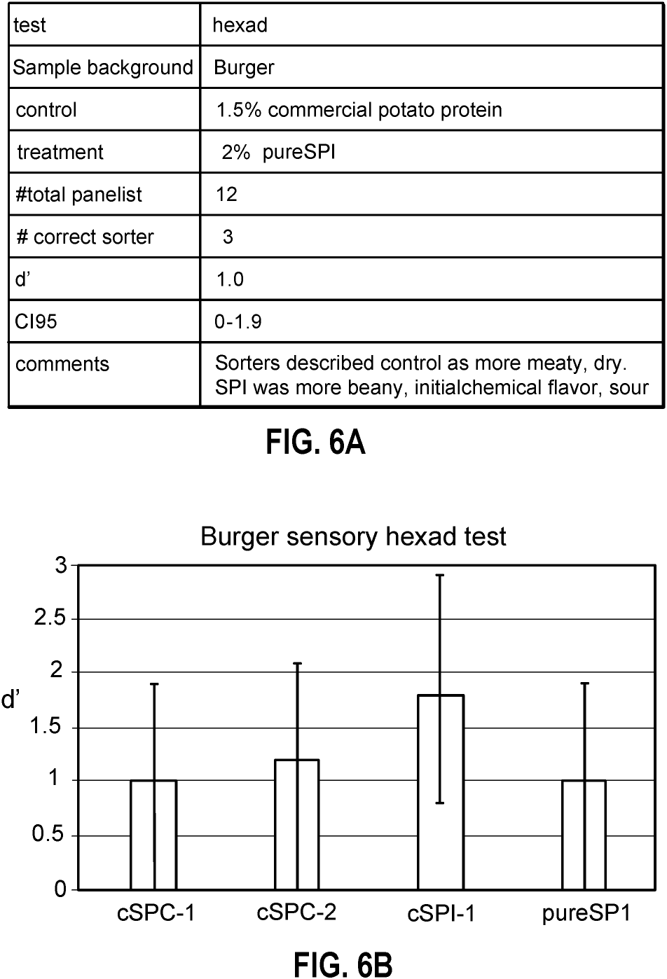

FIGS. 6A and 6B show exemplary data from a hexad discrimination test using a burger product. In this test, a commercial protein (1.5% potato protein) was used as the control, and various commercial proteins and pureSPI (2%) were used as the test conditions.

### Example 12

### SPI-Based Milk Replicas

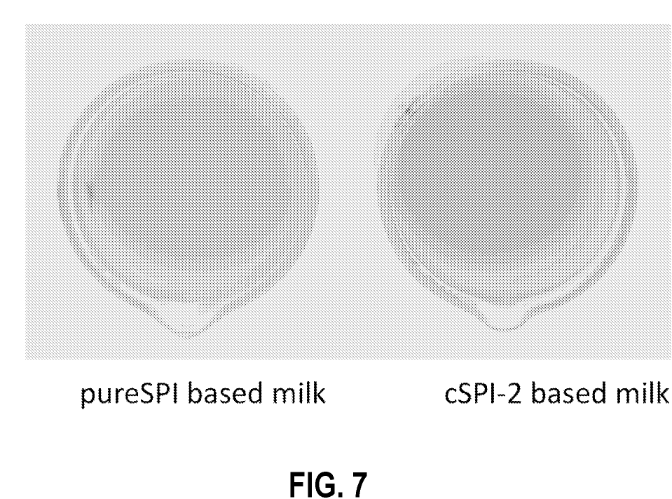

10 g SPI (pureSPI or cSPI-2) was suspended in 300 mL water while stirring. 10 g coconut oil, melted in a beaker incubating in a 40° C. water bath, was added to the SPI suspension. The mixture was stirred vigorously to form a homologous primary emulsion. The emulsion was cooled to ice cold in an ice bucket and sonicated for 4 min to form a stable secondary emulsion. This is the SPI-based milk replica. (FIG. 7)

### Example 13

### Sensory Characterization of SPI-Based Milk Replicas

Standard unspecific hexad tests were performed to evaluate the taste differences between the two SPI-based milks. The panelists were directed to taste 6 samples from amber vials: 3 were pureSPI-based milk replicas and 3 were cSPI-2 based milk replicas. Each of the 12 panelists was asked to sort these samples into 2 groups of 3 and, for each group, specify the sensory criteria that helped them to decide which sample belonged to which group.

Two independent such tests were performed. Six out of twelve panelists in a first test and nine out of twelve panelists in a second test sorted the samples correctly, which represent discriminability index, d’, ranging from 1.7 to 2.4. The panelists determined a moderate to large difference between the groups. According to the correct sorters, the cSPI-2 based milk replica was described as bitter, soy, and beany. The pureSPI based milk replica was described as mild, bland, and almond notes.

The visual differences between the two SPI-based milk replicas were evaluated by an unspecific hexad test. In this test, the panelists were directed to observe 6 samples in clear glass vials: 3 were pureSPI-based milk replicas and 3 were cSPI-2 based milk replicas. Each panelist was asked to sort these samples into 2 groups of 3 and specify which group has a whiter appearance. Of 16 total panelists, 15 sorted the samples correctly, which represents a discriminability index, d′, of 3.3 with 95% confidence interval between 2.2 to 5.0. The panelists concluded there was a moderate difference between the groups, and the pureSPI based milk replica was whiter and cSPI-2 based milk replica was a beige and creamy color.

### Example 14

### Particle Size Characterization

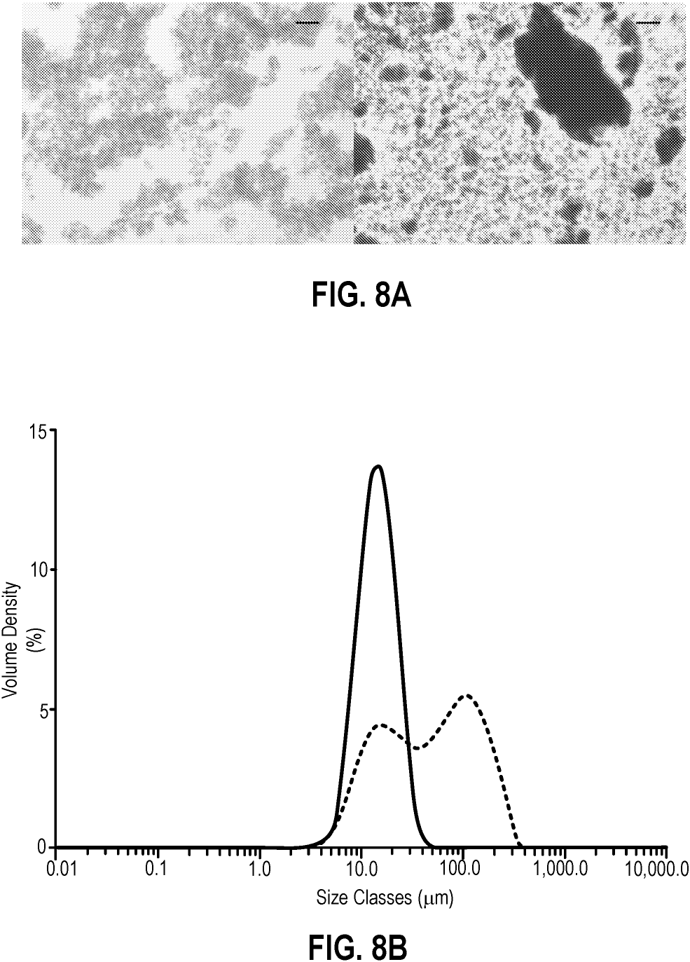

A particle analysis of a SPI precipitate was performed. Microscope images in FIG. 8A show the morphology differences between ethanol precipitated soy protein (left) and acid precipitated soy protein (right). Scale bar is 100 µm. FIG. 8B shows particle size distributions measured with a light scattering instrument (Malvern MasterSizer). The line with the single peak represents ethanol precipitated soy protein and the line with the double peak shows the acid precipitated soy protein, indicating that the ethanol precipitated protein has a more uniform particle size distribution than the acid precipitated protein.

### Example 15

### GCMS Method

Protein size reduction: Protein (e.g., textured vegetable protein, TVP) particle size is reduced to homogeneous powder before GCMS sample preparation. A cryogenic mill (e.g., a SPEX Freezer Mill) is used to mill fine powder without introducing heat.

GCMS sample preparation: Milled protein powders are suspended to either water or flavor broth at 1% w/v concentration. 3 ml of the sample is aliquoted into 20 ml GC vial and crimped.

Sample cooking and volatile extraction: The protein suspension is either uncooked or cooked (150° C., 3 min, 750 rpm in a heated agitator) before headspace sampling. Headspace volatiles are extracted with a SPME fiber (type: DVB/CAR/PDMS) at 50° C.

GCMS data collection: Volatiles are separated on a capillary wax column with temperature ramp from 35° C. to 255° C. Data is collected at 10 Hz with mass range from 20 to 500.

GCMS data Analysis: Data analyses are done by comparing collected data with an internal GCMS database as well as NISt database.

### Example 16

### Preparing PureProtein Using a Heating Step Before Precipitation

In the above examples, pureProtein was prepared by aqueously extracting, precipitating using a solvent, washing, and drying the protein. Precipitation with a solvent such as ethanol forms a homogenous suspension of small particles (average diameter of approximately 10 µm) and centrifugation is used to separate the particles from the solvent. In this example, the extracted material was heated before the precipitation. When the extracted material is heated for up to 20 minutes (e.g., 10 seconds to 20 minutes) at 85° C. -95° C. (e.g., 90° C.) before precipitation, adding the solvent forms a cheese-curd like structure and a visible clear whey fraction with increasing treatment time (e.g., from 1 to 20 minutes). This curd-like precipitate can be easily separated from ethanol extract by filtration, before washing and drying to produce pureProtein. Thus, heating the extracted material before precipitating increased the precipitate structure and may have disrupted intermolecular interactions between the protein and other components, allowing easy recovery of the precipitated material and decreasing small molecule contaminants.

The total protein, fat, ash (remaining inorganic material after incineration), and carbohydrate content (% dry basis) was analyzed for pureSPI and pureSPC products produced as described in Examples 1 and 2, respectively, with a heating step before precipitation, and compared to that of a commercial SPI and two commercial SPCs. As shown in Table 1, heating the extracted material before precipitation decreased the fat and carbohydrate content and increased the protein content in both the pureSPC and pureSPI products. Thus, heating the extracted material before precipitating can improve the final product quality by decreasing small molecule contaminants and/or increasing protein content.

TABLE 1

Protein, fat, ash, and carbohydrate content (%, dry basis) in typical commercial soy proteins and pureProtein process generated soy proteins

| | Protein | Fat | Ash | Carb |
|---|---|---|---|---|
| Commercial SPI | 89.2 | 5.0 | 3.9 | 4.8 |
| pureSPI (no heat) | 91.2 | 0.5 | 6.0 | 2.3 |
| pureSPI (with heat) | 93.5 | 0.2 | 5.3 | 1.0 |
| Commercial SPC1 | 70.0 | 1.0 | 6.8 | 22.2 |
| Commercial SPC2 | 70.3 | 1.0 | 6.9 | 21.6 |
| pureSPC (no heat) | 68.3 | 0.7 | 5.6 | 25.5 |
| pureSPC (with heat) | 73.0 | 0.5 | 5.2 | 21.3 |

### Example 17

### Preparing PureProtein Using Cold Ethanol Precipitation

In an effort to improve functionality and solubility of pureProtein, both the precipitation step and wash steps were performed at cold temperatures. In addition to improving solubility of the resulting protein composition, the cold ethanol precipitation also improves the gelation property. For the protein extraction, 100 g soy flour was resuspended in 900 mL Milli-Q water, and the pH of this 10% slurry was adjusted to pH 8.0 using sodium hydroxide. The slurry was stirred at room temperature for 30 min. The fiber was removed by centrifuging the slurry at 2,000 × g for 5 min at 4° C. The supernatant (~750 mL) was collected and cooled on ice. The pellet was discarded. The proteins in the supernatant were precipitated with cold ethanol. In particular, the ethanol was pre-cooled with liquid nitrogen to — 20° C. and the protein solution was pre-cooled on ice to 4° C. The cold ethanol (750 mL) was slowly added to the protein solution while stirring. The proteins were precipitated out immediately, but the particle size looked very fine. The final temperature was 6° C. due to the heat release by mixing water and ethanol. The mixture was kept on ice for 10 min, then centrifuged at 2,000 × g for 5 min at 4° C. to pellet the precipitated protein. The supernatant was discarded.

The precipitated protein was washed by adding 1 L of 20° C. ethanol to the protein pellet, and blending in a Vitamix blender for 30 s. The mixture then was centrifuged at 2,000 × g for 5 min at 4° C. to pellet the precipitated protein, and the supernatant was discarded. The wet pellet was frozen in liquid nitrogen, and then loaded onto a lyophilizer. The pellet was completely dried after 5 days. The dry protein pellet was blended in blender for 2 min, resulting in an off-white powder. The sample was labeled as cold-precipitated pureSPI.

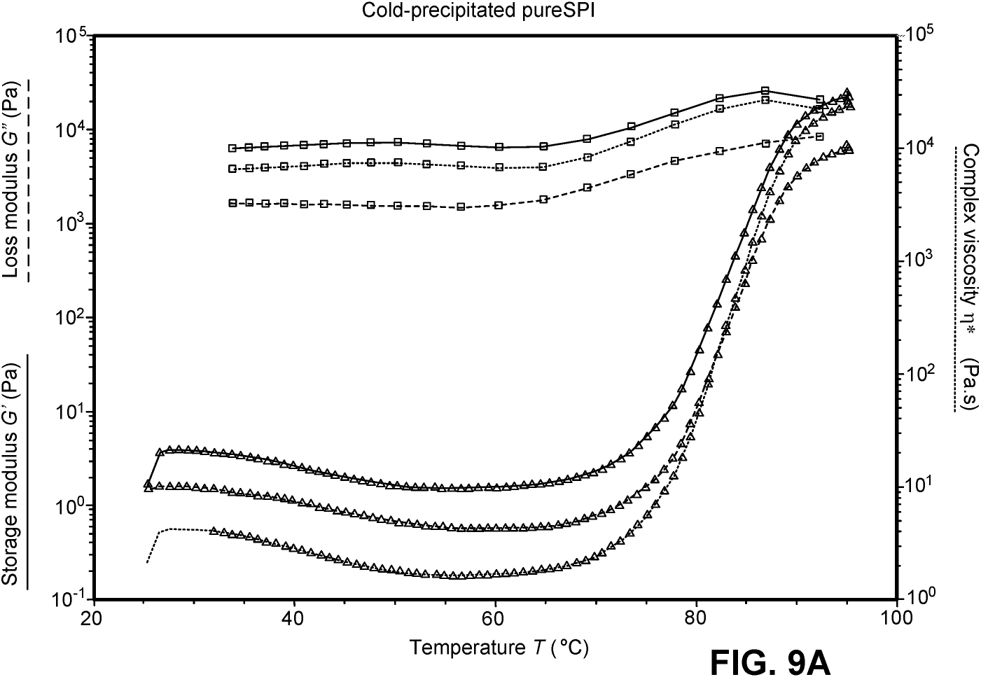

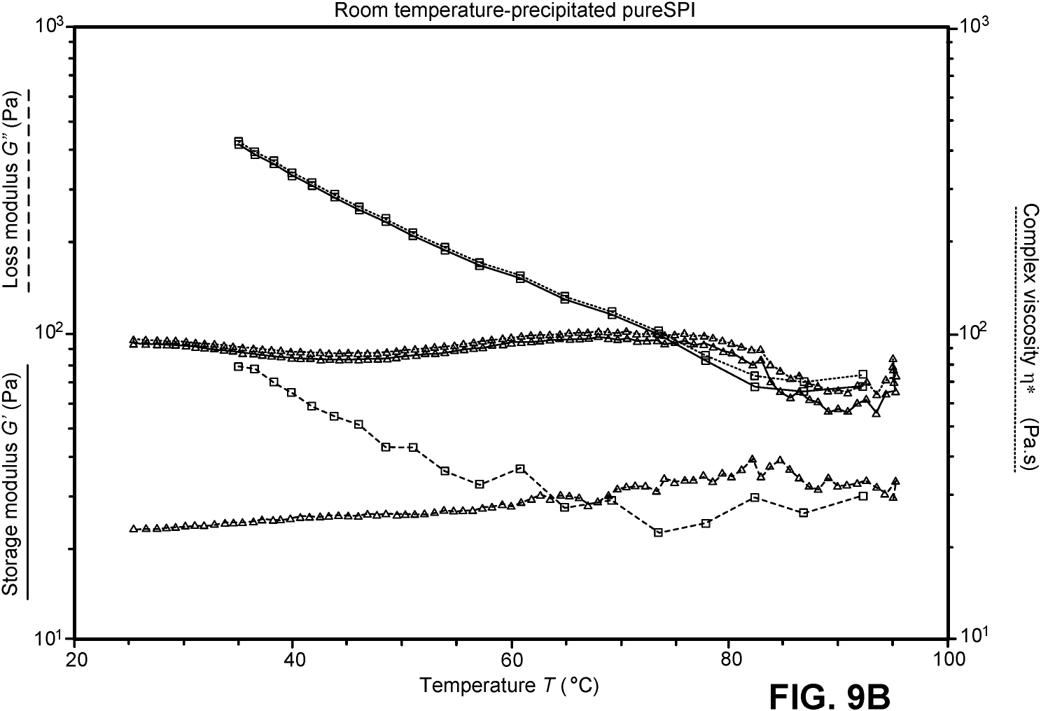

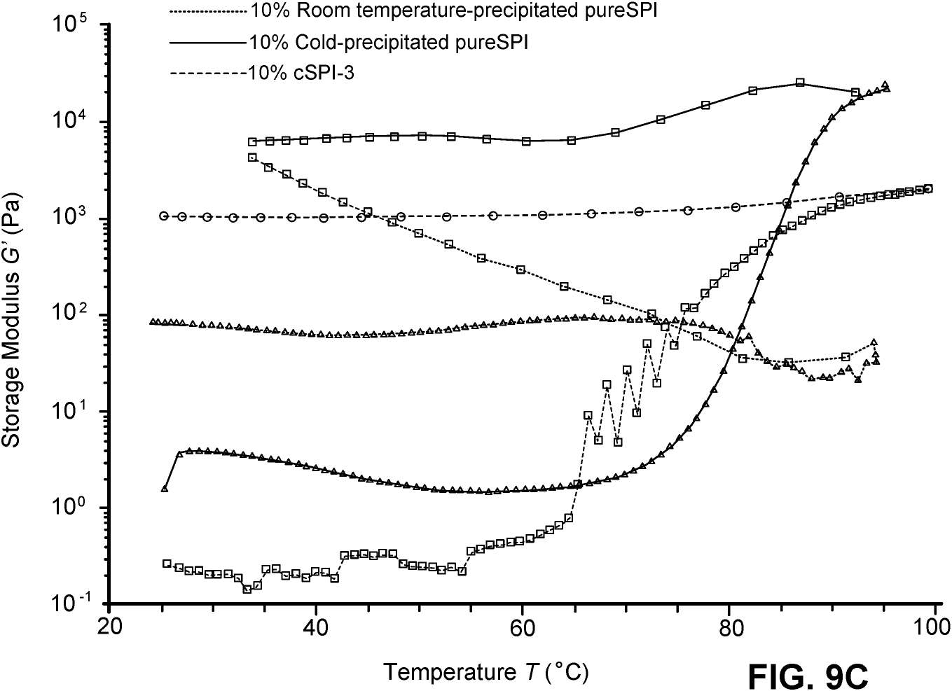

For comparison purposes, this process also was performed in parallel, with all the materials at room temperature. The room temperature-precipitated SPI sample was whiter and fluffier than the cold-precipitated pureSPI, which is similar to typical pureSPI. Both protein materials were tested with an assay to measure temperature-dependent changes in mechanical properties such as storage modulus, loss modulus, and viscosity, using the Discovery Hybrid Rheometer (DHR) with Peltier Plate with temperature control and evaporation covers. Measurements are taken as the sample temperature ramps from 25° C. to 95° C. and then cools down to 40° C. Instrument is set-up and calibrated, sample is loaded and run, and data is processed. The results indicated the cold process preserved greater functionality of the SPI. For example, as shown in FIG. 9A for cold-precipitated pureSPI, the storage modulus, loss modulus, and complex viscosity increased with increasing temperatures. These changes were substantially irreversible as decreasing temperatures did not substantially reduce any of these parameters. In contrast, as shown in FIG. 9B for room temperature-precipitated pureSPI, storage modulus, loss modulus, and complex viscosity were not substantially altered with changes in temperature. FIG. 9C highlights the difference in storage modulus for the pureSPI prepared as in Example 1 and the cold-precipitated pureSPI.

The solubility of the materials also was tested. The room temperature precipitated pureSPI and cold precipitated pureSPI were each resuspended in water as a 1% slurry, after incubation and vortex, the solids was removed by centrifuge at 1,500 x g for 5 min. The total protein concentration in the supernatant was measured. The values were 2.6 mg/mL for room temperature-precipitated pureSPI, and 3.1 mg/mL for cold-precipitated pureSPI.

### Example 18

### Preparing PureProtein Using a Water Wash Before Drying

As described in this example, washing with different percentages of ethanol can increase solubility and create a material with a foaming property. Defatted soy flour was extracted under alkaline conditions at 10% w/w at RT for 30 min. The supernatant was collected by centrifugation, and adjusted to either 1) pH 6, then precipitated with equal volume of 100% EtOH (approximately 47.5% EtOH final) or 2) pH 4.5, isoelectric precipitation (no ethanol). The precipitated solids were collected by centrifugation and dispersed in three pellet volumes wash solvent (0 to 100% EtOH) by blending. In the samples adjusted to pH 6, foaming was observed with wash solvent with 0 to 50% EtOH, with the greatest amount of foam with wash solvent with 0% EtOH. In the samples adjusted to pH 4.5, foaming was observed with wash solvent with 0 to 50% EtOH, with the greatest amount of foam with wash solvent with 5 to 10% EtOH. The washed solids were collected by centrifugation, weighed, and then dried by lyophilization. The wash supernatant protein concentration was measured using a Pierce protein assay.

The washed solids were collected by centrifugation, freeze-dried, and then powderized by blending. In the powder obtained from the samples adjusted to pH 6, the solids were voluminous, white, and soft when 0-10% EtOH was used for washing, whereas the solids were more compact, more yellow, and harder when between 20-70% EtOH was used for washing. At 95-100% EtOH, the solids were voluminous and white, and slightly gritty. In the powder obtained from the samples adjusted to pH 4.5, there was a loss of mass to soluble protein in the wash supernatant (see below), and the material properties (e.g., color and texture) were more similar throughout EtOH range.

Prior to precipitation, the protein concentration of the starting materials was ~30 mg/mL. Soluble protein in the pH 6 supernatant was lower (0.3 mg/mL) than for pH 4.5 (2.2 mg/mL ). It is noted that the wash resuspensions were subjected to high shear (blender), which may contribute to resolubilization.

For the samples in the pH 6 group, samples washed with 95% EtOH and above have very low resolubilized protein, whereas 70% EtOH resolubilizes moderately at 4.4 mg/mL, and lower concentrations of ethanol resolubilizes up to 15.1 mg/mL.

For the samples in the pH 4.5 group, samples washed with 50% EtOH and above have very low resolubilized protein, whereas 30% EtOH has moderate at 4.7 mg/mL, and lower concentrations of ethanol have largely soluble protein (21-31 mg/mL ).

For ethanol-precipitated protein, high-shear washing with 50% ethanol and lower can recover partially soluble protein. For acid-precipitated protein, high-shear washing with sodium hydroxide and 0-50% ethanol can tune protein solubility.

The mass of the washed wet (centrifuged washed wet pellet) and washed dry pellets (same pellet, after lyophilization) was measured for the pH 6 and pH 4.5 groups, and the dry matter percentage (DM%) in washed pellet (mass dry / mass wet) was determined. Overall, the pellets from the pH 6 group have a lower DM% than pH 4.5 precipitated group, suggesting they have a higher solvent-binding capacity and/or are less dense. For pellets in the pH 6 group, at higher ethanol concentrations 70% and above, the pellets exposed to higher ethanol concentrations also have a lower DM% (higher solvent-holding capacity / lower density).

For pH 4.5 precipitations, at ethanol wash concentrations 20% and below, both wet and dry pellet masses decrease as protein solubility increases and is lost to the liquid waste stream. A high DM% at 0% water is likely due to measurement error at very low masses. For pH 4.5 precipitation, wash concentrations of at 50% or above, the wet pellet mass slightly decreases though dry pellet mass stays the same. This suggests that the pellets exposed to ethanol concentrations around 30-50% have a higher solvent-binding capacity or decreased density, and at higher concentrations of 70% ethanol and above, the solvent-binding capacity diminishes

### Example 19

### Resolubilizing PureProtein

This example describes post-processing steps including pH excursion to improve the solubility of the final protein composition and enzymatic treatment using protein glutaminase to improve the solubility and make the final protein composition stable when added to an acidic solution.

To re-solubilize the pureProtein precipitated by ethanol, a pH excursion was performed. The following procedure was followed: pureSPI powder was resuspended in water to make a 0.5% slurry, then sonicated or vortexed to disperse the solid in the water. 2 M NaOH solution was added to the mixture while stirring, and the pH was monitored with a pH test strip. When the pH increased to 9, most of the solid was dissolved; and when the pH reached 10, the solution turned clear and only a minimal amount of solids was left. The total protein concentration was measured with Pierce 660 nm Assay (mg/mL) at pH 7, 8, 9, 10, and 11. PureSPI started to be solubilized when pH reached 9, and most of the pureSPI was solubilized when pH was greater than 10. After solubilization, pureSPI solution can be neutralized or its pH can be adjusted to a target pH (e.g., for use in a food product).

In other experiments, pureProtein was resolubilized by treating with a protein glutaminase (Amano Enzyme (“Amano” 500, Lot#: PGP0451331KR). Four experiments were conducted. Approximately 2 g pureSPI was resuspended in 20 mL Milli-Q water, using a sonicator to completely disperse the SPI into water and then 10 mg protein glutaminase powder was added into the suspension, heated to 50° C. for 1.5 hr while stirring. The slurry was diluted to 50 mL with Milli-Q water, and then 2 g melted hydrogenated coconut oil was added and the mixture was homogenized for 1 min using sonication at full power. The resulting milk replica was poured into freshly prepared hot espresso. After the pureSPI and hot coffee were evenly mixed, no protein aggregation or precipitation was observed.

### Example 20

### Sodium Levels in PureProtein

This example examines sodium levels in typical commercial soy proteins and pureProtein process generated soy proteins. As shown in FIG. 10, pureSPC has lower levels of sodium than two commercial SPCs (cSPC-1 and cSPC-3), and pureSPI has significantly lower levels of sodium than two commercial SPIs (cSPI-1 and cSPI-3). FIG. 10 is a plot of the sodium levels in various SPIs.

### Example 21

### Isoflavone, Saponin, and Phospholipid Content

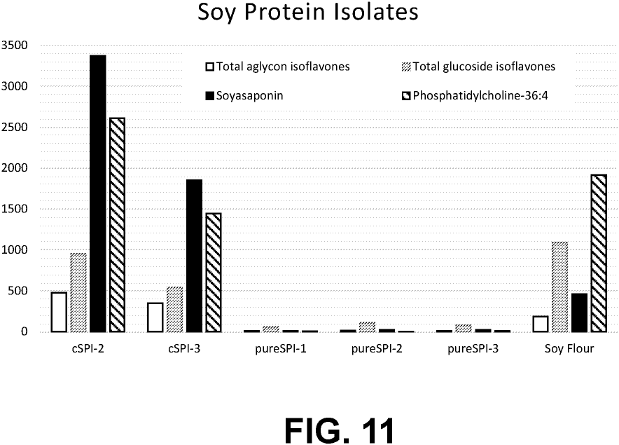

The isoflavone, saponin (using soyasaponin as an indicator of overall saponin content), and phospholipid (using phosphatidylcholine-36:4 as an indicator of overall phospholipid content) content of soy flour, two commercial SPIs (cSPI-2 and cSPI-3), and three replicates of pureSPI made according to Example 16 was evaluated using the method of Example 15. The results are shown in FIG. 11, indicating that the pureSPI protein has lower content of aglycon isoflavones, glucoside isoflavones, soyasaponin, and phosphatidylcholine-36:4 than commercial SPIs and soy flour.

### Example 22

### Flavor of Texturized SPC

To assess the flavor of SPC (e.g., the SPC from Example 2), the SPC is extruded into texturized SPC. Approximately 10 g of the resultant texturized SPC is hydrated in 100 ml water, cooked at 80° C. for 30 mins, cooled (e.g., to room temperature), and assessed via a trained descriptive panel using the Spectrum™ method. The sample is described as having low intensity of off-flavors— overall aromatic impact <4.5, vegetable complex (<3.5), oxidized/rancid (<0.2), sweet fermented (<0.5), astringent (<2), and bitter (<2).

### Example 23

### Flavor of SPC

To assess the flavor of the SPC (e.g., the SPC from Example 2), 2 g of the SPC is hydrated in 100 ml water and assessed via a trained descriptive panel using the Spectrum™ method. The sample is described as having low intensity of oxidized/rancid, cardboard, astringent, and bitter off-flavors (<8).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A protein composition comprising:
at least 50% by dry weight of a plurality of plant proteins, fungal proteins, algal proteins, bacterial proteins, protozoan proteins, invertebrate proteins, or a combination thereof,
wherein the protein composition is a low color protein composition.

2. A protein composition comprising:
at least 50% by dry weight of a plurality of plant proteins, fungal proteins, algal proteins, bacterial proteins, protozoan proteins, invertebrate proteins, or a combination thereof;
less than 1.0% by dry weight of lipids.

3. A protein composition produced by a method comprising:
(a) adding an aqueous solution to a source protein composition to form a solution of solubilized protein;
(b) optionally removing solids from the solution of solubilized protein;
(c) optionally heating the solution of solubilized protein;
(d) optionally adjusting the pH of the solution of solubilized protein to about 4.0 to about 9.0;
(e) optionally cooling the solution of solubilized protein to about 0° C. to about 10° C.;
(f) adding an organic solvent to the solution of solubilized protein to form a solid phase and a liquid phase;
(g) separating the solid phase from the liquid phase to form the protein composition;
(h) optionally washing the protein composition with a wash solvent; and
(i) optionally treating the protein composition,
wherein the protein composition comprises at least at least 50% by dry weight of a plurality of plant proteins, fungal proteins, algal proteins, bacterial proteins, protozoan proteins, invertebrate proteins.

4. The protein composition of any one of claims 1-3, wherein the protein composition comprises at least about 90% by dry weight of the plurality of plant proteins, fungal proteins, algal proteins, bacterial proteins, protozoan proteins, invertebrate proteins, or the combination thereof.

5. The protein composition of any one of claims 1-3, wherein the protein composition comprises at least about 91% by dry weight of the plurality of plant proteins, fungal proteins, algal proteins, bacterial proteins, protozoan proteins, invertebrate proteins, or the combination thereof.

6. The protein composition of any one of claims 4-5, wherein the protein composition is a protein isolate.

7. The protein composition of claim 6, wherein the protein composition comprises less than 8% by dry weight of insoluble carbohydrates.

8. The protein composition claim 6 or claim 7, wherein the protein composition is a low flavor protein composition.

9. The protein composition of any one of claims 6-8, wherein the protein composition has an isoflavone content of less than about 125 ppm.

10. The protein composition of any one of claims 6-9, wherein the protein composition has a saponin content of less than about 75 ppm.

11. The protein composition of any one of claims 6-10, wherein the protein composition has a phospholipid content of less than about 500 ppm.

12. The protein composition of any one of claims 6-11, wherein the protein composition has a phospholipid content of less than about 25 ppm.

13. The protein composition of any one of claims 6-12, wherein the protein composition has a phospholipid content of less than about 5 ppm.

14. The protein composition of any one of claims 1-3, wherein the protein composition comprises about 60% to about 80% by dry weight of the plurality of plant proteins, fungal proteins, algal proteins, bacterial proteins, protozoan proteins, invertebrate proteins, or the combination thereof.

15. The protein composition of claim 14, wherein the protein composition comprises about 65% to about 75% by dry weight of the plurality of plant proteins, fungal proteins, algal proteins, bacterial proteins, protozoan proteins, invertebrate proteins, or the combination thereof.

16. The protein composition of claim 14, wherein the protein composition is a protein concentrate.

17. The protein composition of any one of claims 1-16, wherein the protein composition comprises less than 0.8% by dry weight of lipids.

18. The protein composition of any one of claims 1-17, wherein the protein composition comprises less than 0.4% by dry weight of lipids.

19. The protein composition of any one of claims 1-18, wherein the protein composition has a luminance of at least 86 on a scale from 0 (black control value) to 100 (white control value).

20. The protein composition of any one of claims 1-19, wherein the protein composition has a luminance of at least 90 on a scale from 0 (black control value) to 100 (white control value).

21. The protein composition of any one of claims 1-20, wherein the protein composition has a chroma value of less than 14.

22. The protein composition of any one of claims 1-21, wherein the protein composition has a chroma value of less than 8.

23. The protein composition of any one of claims 1-22, wherein the plurality of plant proteins, fungal proteins, algal proteins, bacterial proteins, protozoan proteins, invertebrate proteins, or the combination thereof comprises at least 90% plant proteins.

24. The protein composition of any one of claims 1-22, wherein the plurality of plant proteins, fungal proteins, algal proteins, bacterial proteins, protozoan proteins, invertebrate proteins, or the combination thereof comprises at least 90% fungal proteins.

25. The protein composition of any one of claims 1-22, wherein the plurality of plant proteins, fungal proteins, algal proteins, bacterial proteins, protozoan proteins, invertebrate proteins, or the combination thereof comprises at least 90% algal proteins.

26. The protein composition of any one of claims 1-25, wherein when cooked in a solution comprising a reducing sugar, a sulfur-containing amino acid, and a heme-containing protein, a 1% (w/v) of the protein composition produces one or more volatile compounds associated with the aroma and/or taste of meat.

27. The protein composition of any one of claims 1-26, wherein when assessed by a trained descriptive panel using the Spectrum method, the protein composition is described as having low intensity of one or more of: oxidized/rancid flavor, cardboard flavor, astringent flavor, bitter flavor, vegetable complex flavor, and sweet fermented flavor.

28. The protein composition of any one of claims 1-27, wherein when assessed by a trained descriptive panel using the Spectrum method, the protein composition is described as having low intensity of one or more of: beany flavor, fatty flavor, green flavor, pea flavor, earthy flavor, hay-like flavor, grassy flavor, rancid flavor, leafy flavor, cardboard flavor, acrid flavor, pungent flavor, medicinal flavor, metallic flavor, and brothy flavor.

29. The protein composition of any one of claims 1-28, wherein when assessed by a trained panel, the protein composition has a discriminability index of at least 1.0.

30. The protein composition of any one of claims 1-28, wherein when assessed by a trained panel, the protein composition has a discriminability index of at least 2.0.

31. The protein composition of any one of claims 1-30, wherein the protein composition is in the form of a solution, suspension, or emulsion.

32. The protein composition of any one of claims 1-30, wherein the protein composition is in the form of a solid or a powder.

33. A food product comprising the protein composition of any one of claims 1-32.

34. A method for producing a protein composition, the method comprising:
(a) adding an aqueous solution to a source protein composition to form a solution of solubilized protein;
(b) optionally removing solids from the solution of solubilized protein;
(c) optionally heating the solution of solubilized protein;
(d) optionally adjusting the pH of the solution of solubilized protein to about 4.0 to about 9.0;
(e) optionally cooling the solution of solubilized protein to about 0° C. to about 10° C.;
(f) adding an organic solvent to the solution of solubilized protein to form a solid phase and a liquid phase;
(g) separating the solid phase from the liquid phase to form the protein composition;
(h) optionally washing the protein composition with a wash solvent; and
(i) optionally resolubilizing the protein composition,
wherein the protein composition comprises at least at least 50% by dry weight of a plurality of plant proteins, fungal proteins, algal proteins, bacterial proteins, protozoan proteins, invertebrate proteins.

35. The method of claim 34, wherein step (a) is performed at a pH of about 7.0 to about 10.0.

36. The method of any one of claims 34-35, wherein step (d) comprises adjusting the pH of the solution of solubilized protein to about 6.0 to about 7.0.

37. The method of any one of claims 34-36, wherein step (f) comprises adding the organic solvent to a final concentration of about 40% to about 70% (v/v).

38. The method of any one of claims 34-37, wherein at the beginning of step (f), the organic solvent has a temperature of about -20° C. to about 10° C.

39. The method of any one of claims 34-38, wherein step (e) comprises cooling the solution of solubilized protein to a temperature of about 0° C. to about 4° C.

40. The method of any one of claims 34-39, wherein step (c) comprises heating the solution of solubilized protein at a temperature of about 85° C. to about 95° C.

41. The method of any one of claims 34-40, wherein the organic solvent is selected from the group consisting of ethanol, methanol, propanol, isopropyl alcohol, and acetone.

42. The method of any one of claims 34-41, wherein the organic solvent is ethanol.

43. The method of any one of claims 34-42, wherein the wash solvent is an organic wash solvent.

44. The method of any one of claims 34-42, wherein the wash solvent is an aqueous solution.

45. The method of any one of claims 34-42, wherein the wash solvent is a mixture of an aqueous solution and an organic wash solvent.

46. The method of any one of claims 34-45, wherein the treating comprises resolubilizing the protein composition to a concentration of about 1.5 to about 50 mg/mL.

47. The method of any one of claims 34-46, wherein the treating comprises resolubilizing at least a portion of the protein composition at a pH of at least 8.0.

48. The method of any one of claims 34-47, wherein the treating comprises resolubilizing at least a portion of the protein composition using an enzyme.

49. The method of claim 48, wherein the enzyme is a protein deamidase.

50. The method of any one of claims 34-49, wherein, when cooked in water, a 1% (w/v) suspension of the protein composition by dry weight of the protein composition produces no more than 90% of the amount of one or more volatile compounds in a set of volatile compounds produced by cooking a 1% (w/v) suspension of the source protein composition (by dry weight of the source protein composition).

51. The method of any one of claims 34-50, wherein, when cooked in water, a 1% (w/v) suspension of the protein composition by dry weight of the protein composition produces no more than 50% of the amount of one or more volatile compounds in a set of volatile compounds produced by cooking a 1% (w/v) suspension of the source protein composition (by dry weight of the source protein composition).

52. The method of any one of claims 34-51, wherein the protein composition produces no more than 90% of the amount of one or more volatile compounds in a set of volatile compounds produced by the source protein composition by solvent-assisted flavor extraction (SAFE).

53. The method of any one of claims 34-52, wherein the protein composition produces no more than 50% of the amount of one or more volatile compounds in a set of volatile compounds produced by the source protein composition by SAFE.

54. The method of any one of claims 50-53, wherein the set of volatile compounds comprises a volatile compound in any one of volatile sets 1-10.

55. The method of any one of claims 50-53, wherein the set of volatile compounds is any one of volatile sets 1-10.

56. The method of any one of claims 50-53, wherein the set of volatile compounds is selected from the group consisting of volatile set 1, volatile set 2, volatile set 3, volatile set 4, volatile set 5, volatile set 6, volatile set 7, volatile set 8, volatile set 9, volatile set 10, and combinations thereof.

57. The method of any one of claims 34-56, wherein the protein composition has a saponin content that is less than 50% of the saponin content of the source protein composition.

58. The method of any one of claims 34-57, wherein the protein composition has an isoflavone content that is less than 50% of the isoflavone content of the source protein composition.

59. The method of any one of claims 34-58, wherein the protein composition has a phospholipid content that is less than 50% of the phospholipid content of the source protein composition.

60. The method of any one of claims 34-59, wherein the protein composition has a lipid content that is less than 50% of the lipid content of the source protein composition.

61. The method of any one of claims 34-60, wherein the protein composition has a phenolic acid content that is less than 50% of the phenolic acid content of the source protein composition.

62. The method of any one of claims 34-61, wherein the protein composition has a flavor compounds content that is less than 50% of the flavor compounds content of the source protein composition, wherein the flavor compounds are selected from the group consisting of elected from aldehydes, ketones, esters, alcohols, pyrazines, pyranones, acids, sulfur compounds, terpenes, furans, alkanes, alkenes, and combinations thereof.

63. A food product comprising a protein composition produced by the method of any one of claims 34-62.

64. A method of extracting small molecules from a protein source composition, the method comprising:
(a) adding an aqueous solution to a source protein composition to form a solution of solubilized protein;
(b) optionally removing solids from the solution of solubilized protein;
(c) optionally heating the solution of solubilized protein;
(d) optionally adjusting the pH of the solution of solubilized protein to about 4.0 to about 9.0;
(e) optionally cooling the solution of solubilized protein to about 0° C. to about 10° C.;
(f) adding an organic solvent to the solution of solubilized protein to form a solid phase and a liquid phase;
(g) separating the solid phase from the liquid phase to form a solution enriched in small molecules.

65. A food product comprising:
a fat;
optionally one or more flavor precursor compounds; and
at least 10% by dry weight of a protein composition, wherein the protein composition is the protein composition of any one of claims 1-32.

66. A food product comprising:
a fat;
optionally one or more flavor precursor compounds; and
at least 10% by dry weight of a protein composition, wherein the protein composition is a protein composition produced by the method of any one of claims 34-62.

67. The food product of any one of claims 65-66, wherein the food product is a meat replica.

68. The food product of any one of claims 65-67, wherein the food product is plant-based.

69. The food product of any one of claims 65-68, wherein the food product contains less than 10% by weight animal products.

70. A method for preparing a food product, the method comprising:
combining a fat, one or more optional flavor precursor compounds, and a protein composition, wherein the protein composition is the protein composition of any one of claims 1-32.

71. A method for preparing a food product, the method comprising:
combining a fat, one or more optional flavor precursor compounds, and a protein composition, the protein composition produced by the method by the method of any one of claims 34-62.

72. A method for reducing perceived protein source flavor in a food product, the method comprising:
combining a fat, one or more flavor precursor compounds and a protein composition, the protein composition produced by the method of any one of claims 34-62,
wherein at least 5% by weight of the protein content of the food product comprises the protein composition, thereby reducing perceived protein source flavor in a food product, as compared to a food product having a similar protein content but lacking the protein composition.

73. A method of evaluating a protein composition for effect on flavor in a food product, the method comprising:
determining that a level of one or more volatile compounds in a set of volatile compounds of a first protein composition from a protein source is higher than the level of the one or more volatile compounds of a second protein composition from the protein source; and
determining that the second protein composition is superior to the first protein composition for use in a food product.

74. A method of evaluating a protein composition for effect on flavor in a food product, the method comprising:
determining that a level of one or more volatile compounds in a set of volatile compounds of a source protein composition from a protein source is higher than the level of the one or more volatile compounds of a protein composition from the protein source; and
determining that the protein composition is superior to the source protein composition for use in a food product.

75. The method of claim 73, wherein the second protein composition is the protein composition of any one of claims 1-32.

76. The method of claim 74, wherein the protein composition is the protein composition of any one of claims 1-32.

77. The method of any one of claims 73-76, wherein the food product is the food product of any one of claims 65-69.

78. The method of any one of claims 73-77, wherein the set of volatile compounds comprises a volatile compound from any one of volatile sets 1-10.

79. The method of any one of claims 73-77, wherein the set of volatile compounds is any one of volatile sets 1-10.

80. The method of any one of claims 73-77, wherein the set of volatile compounds is selected from the group consisting of volatile set 1, volatile set 2, volatile set 3, volatile set 4, volatile set 5, volatile set 6, volatile set 7, volatile set 8, volatile set 9, volatile set 10, and combinations thereof.

81. The method of any one of claims 73-80, wherein the protein source is soy.

82. A method of reducing flavor in a protein composition, the method comprising:
(a) determining a level of one or more volatile compounds in a set of volatile compounds of a first protein composition from a protein source;
(b) preparing a second protein composition from the protein source, wherein preparing the second protein composition comprises reducing the amount of one or more components of the protein source that are included in the second protein composition; and
(c) determining that a level of one or more volatile compounds in a set of volatile compounds from the second protein composition is lower than the level of the one or more volatile compounds in a set of volatile compounds in the first protein composition.

83. A method of determining a cause of flavor in a protein composition, the method comprising:
(a) determining a level of one or more volatile compounds in a set of volatile compounds of a first protein composition from a protein source;
(b) providing a second protein composition from the protein source, wherein the second protein composition comprises a decreased amount of one or more components of the protein source;
(c) determining that a level of one or more volatile compounds in a set of volatile compounds from the second protein composition is lower than the level the of one or more volatile compounds in a set of volatile compounds in the first protein composition; and
(d) identifying the one or more components of the protein course to be a cause of flavor in the protein composition.

84. The method of claim 82 or claim 83, wherein the second protein composition is the protein composition of any one of claims 1-32.

85. The method of any one of claims 82-84, wherein the set of volatile compounds comprises a volatile compound from any one of volatile sets 1-10.

86. The method of any one of claims 82-84, wherein the set of volatile compounds is any one of volatile sets 1-10.

87. The method of any one of claims 82-84, wherein the set of volatile compounds is selected from the group consisting of volatile set 1, volatile set 2, volatile set 3, volatile set 4, volatile set 5, volatile set 6, volatile set 7, volatile set 8, volatile set 9, volatile set 10, and combinations thereof.

88. The method of any one of claims 82-87, wherein the protein source is soy.

89. The method of any one of claims 82-88, wherein the component of the protein source that is decreased comprises lipids.

90. The method of any one of claims 82-89, wherein the component of the protein source that is decreased comprises a fatty acid, a wax, a sterol, a monoglyceride, a diglyceride, a triglyceride, a sphingolipid, phospholipid, or a combination thereof.

91. The method of any one of claims 82-90, wherein the component of the protein source that is decreased comprises phospholipids.

92. A milk replica comprising:
an emulsion of a fat, water, and the protein composition of any one of claims 1-32.

93. The milk replica of claim 92, wherein the fat is present in the milk replica in an amount of about 0.01% to about 5% of the milk replica.

94. The milk replica of claim 93, wherein the fat is selected from the group consisting of corn oil, olive oil, soy oil, peanut oil, walnut oil, almond oil, sesame oil, cottonseed oil, rapeseed oil, canola oil, safflower oil, sunflower oil, flax seed oil, palm oil, palm kernel oil, coconut oil, babassu oil, shea butter, mango butter, cocoa butter, wheat germ oil, rice bran oil, and combinations thereof.

95. The milk replica of claim 92 or claim 93, wherein the emulsion is stable when added to a liquid with a temperature of between about 50° C. to about 85° C.

96. The milk replica of claim 95, wherein the liquid is coffee, espresso, or a combination thereof.
